(19) United States
(12) Patent Application Publication
(10) Pub. No.: US 2016/0209428 A1
(43) Pub. Date: Jul. 21, 2016

(54) DIAGNOSTIC AND PREDICTIVE METABOLITE PATTERNS FOR DISORDERS AFFECTING THE BRAIN AND NERVOUS SYSTEM

(71) Applicant: THE REGENTS OF THE UNIVERSITY OF CALIFORNIA, Oakland, CA (US)

(72) Inventors: Robert K. Naviaux, San Diego, CA (US); Dewleen Baker, La Jolla, CA (US)

(21) Appl. No.: 14/913,319

(22) PCT Filed: Aug. 21, 2014
(86) PCT No.: PCT/US14/52197

§ 371 Date: Feb. 19, 2016

Related U.S. Application Data
(60) Provisional application No. 61/868,476, filed on Aug. 21, 2013.

(51) Int. Cl. G01N 33/68; A61K 31/185

(52) U.S. Cl. CPC G01N 33/6896; A61K 31/185; G01N 2570/00; G01N 2800/301; G01N 2800/50; G01N 2800/28

(57) ABSTRACT

The disclosure provides for methods that integrate metabolic testing results from a patient's biological sample for predicting or diagnosing neurological disease and disorders.

### CROSS REFERENCE TO RELATED APPLICATIONS

This application claims priority under 35 U.S.C. §119 from Provisional Application Ser. No. 61/868,476, filed Aug. 21, 2013, the disclosure of which is incorporated herein by reference.

### FIELD OF THE INVENTION

This disclosure relates to biomarkers useful for diagnosing and predicting develop of various neurological disorders and psychiatric disorders.

### BACKGROUND

The importance of evaluating and identifying people with psychiatric illness or neurological deficits is important for assessing their abilities or risk for carrying out certain activities including, for example, purchasing and handling fire arms, driving, flying, and the like. In addition, identifying people who are at risk or have a psychiatric illness or neurological disorder can assist in identifying appropriate therapies or slow the advancement of disease development.

For example, the cost of treating post-traumatic stress disorder (PTSD) for soldiers participating in Iraq and Afghanistan from 2003-2010 has been approximately $1.4 billion. Approximately 21% of soldiers have been observed to develop PTSD after deployment to Iraq or Afghanistan. Methods are needed to identify subjects having or predisposed to developing various neurological or psychiatric disorders such as PTSD would be useful to reduce risk and identify therapies.

### SUMMARY

The disclosure provides methods for diagnosing, predicting, or assessing risk of developing one or more psychiatric or neurological disease, conditions or disorder, and/or diseases, conditions, and disorders associated with cell danger response (CDR), inflammation, neuroinflammation, and/or degeneration such as neurodegeneration.

Among the diseases and disorder are pervasive developmental disorder not otherwise specified, non-verbal learning disabilities, autism, autism spectrum disorders, attention deficit hyperactivity disorder (ADHD), anxiety disorders, post-traumatic stress disorder (PTSD), traumatic brain injury (TBI), social phobia, generalized anxiety disorder, social deficit disorders, schizotypal personality disorder, schizoid personality disorder, schizophrenia, cognitive deficit disorders, dementia, and Alzheimer's Disease in a subject.

In some embodiments, the methods include detecting an amount of each of a plurality of metabolites in a biological sample obtained from the subject, each of the plurality of metabolites being in one of a group of metabolic pathways, such as a set of metabolic pathways the alteration of which is indicative of the disease, condition, or disorder.

In some embodiments, the plurality of metabolites includes at least 2, 3, 4, 5, 6, 7, 8, 9, 10, 11, 12, 13, 14, 15, or 16 metabolites. In some examples, the plurality of metabolites includes at least 8 metabolites and/or includes one, two, or more metabolites in each of at least eight pathways.

In some embodiments, the group of metabolic pathways is selected from the group of pathways consisting of: a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, and non-gonadal steroid metabolic pathway; a pyrimidine metabolic pathway; a 3- and 4-carbon amino acid metabolic pathway; a branched chain amino acid metabolic pathway; a tryptophan, kynurenine, serotonin, melatonin metabolic pathway; a tyrosine and phenylalanine metabolic pathway; a S-adenosylmethionine (SAM), S-adenosylhomocysteine (SAH), methionine, cysteine, and glutathione metabolic pathway; an eicosanoid and resolvin metabolic pathway; a pentose phosphate and gluconate metabolic pathway; a vitamin A and carotenoid metabolic pathway; a glycolysis metabolic pathway; a Kreb's cycle metabolic pathway; and a Vitamin B3 (−Niacin, NAD+) metabolic pathway.

In some embodiments, the group of metabolic pathways includes one or more of the metabolic pathways set forth in Table 1.

In some embodiments, the methods further include comparing the amounts of metabolites so detected with normal or control amounts of the metabolites.

In some embodiments, the methods involve determining, based on the amounts of metabolites so detected, whether respective pathways containing the metabolites are altered in the sample or the subject. In some aspects, the alteration (e.g., elevation or reduction or the elevation or reduction to a significant degree) of at least two metabolites indicates that the pathway is altered.

In some embodiments, the amounts so detected and/or determination of alterations in pathways, indicate that the subject has or is at risk for developing the disease or condition. For example, in some embodiments, the amounts of the plurality, e.g., at least 8, metabolites so determined or detected, indicate a likelihood that the subject is at risk of having or developing the disease or disorder.

In one embodiment, each of said plurality, e.g., at least 8, metabolites is in a metabolic pathway selected from the group of metabolic pathways consisting of a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, and non-gonadal steroid metabolic pathway; a pyrimidine metabolic pathway; a 3- and 4-carbon amino acid metabolic pathway; a branched chain amino acid metabolic pathway; a tryptophan, kynurenine, serotonin, melatonin metabolic pathway; a tyrosine and phenylalanine metabolic pathway; a S-adenosylmethionine (SAM), S-adenosylhomocysteine (SAH), methionine, cysteine, and glutathione metabolic pathway; an eicosanoid and resolvin metabolic pathway; a pentose phosphate and gluconate metabolic pathway; and a vitamin A and carotenoid metabolic pathway.

In some embodiments, the plurality, e.g., at least 8, metabolites comprise a metabolite in each of the following metabolic pathways: a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, and non-gonadal steroid metabolic pathway; a pyrimidine metabolic pathway; a 3- and 4-carbon amino acid metabolic pathway; a branched chain amino acid metabolic pathway; a tryptophan, kynurenine, serotonin, melatonin metabolic pathway; a tyrosine and phenylalanine metabolic pathway; a S-adenosylmethionine (SAM), S-adenosylhomocysteine (SAH), methionine, cysteine, and glutathione metabolic pathway; an eicosanoid and resolvin metabolic pathway; a pentose phosphate and gluconate metabolic pathway; and a vitamin A and carotenoid metabolic pathway.

In some embodiments, each of said plurality, e.g., at least 8, is in a metabolic pathway selected from the group of metabolic pathways consisting of a phospholipid metabolic pathway; a purine metabolic pathway; a sphingolipid metabolic pathway; a cholesterol metabolic pathway; a pyrimidine metabolic pathway; a S-adenosylmethionine (SAM), S-adenosylhomocysteine (SAH), methionine, cysteine, and glutathione metabolic pathway; a microbiome metabolic pathway; a Kreb's Cycle metabolic pathway; a glycolysis metabolic pathway; and a Vitamin B3 (−Niacin, NAD+) metabolic pathway. In another embodiment, the at least 8 metabolites comprise a metabolite in each of the following metabolic pathways a phospholipid metabolic pathway; a purine metabolic pathway; a sphingolipid metabolic pathway; a cholesterol metabolic pathway; a pyrimidine metabolic pathway; a S-adenosylmethionine (SAM), S-adenosylhomocysteine (SAH), methionine, cysteine, and glutathione metabolic pathway; a microbiome metabolic pathway; a Kreb's Cycle metabolic pathway; a glycolysis metabolic pathway; and a Vitamin B3 (−Niacin, NAD+) metabolic pathway.

In some embodiments, each of the plurality, e.g., at least 8, metabolites is in a metabolic pathway selected from the group of metabolic pathways consisting of a phospholipid metabolic pathway; a purine metabolic pathway; a sphingolipid metabolic pathway; a cholesterol cortisol, and/or non-gonadal steroid metabolic pathway; a pyrimidine metabolic pathway; a S-adenosylmethionine (SAM), S-adenosylhomocysteine (SAH), methionine, cysteine, and glutathione metabolic pathway; and a microbiome metabolic pathway.

In some embodiments, the plurality, e.g., at least 8, metabolites comprise a metabolite in each of the following metabolic pathways: a phospholipid metabolic pathway; a purine metabolic pathway; a sphingolipid metabolic pathway; a cholesterol cortisol, and/or non-gonadal steroid metabolic pathway; a pyrimidine metabolic pathway; a S-adenosylmethionine (SAM), S-adenosylhomocysteine (SAH), methionine, cysteine, and glutathione metabolic pathway; and a microbiome metabolic pathway.

In some embodiments of any of the foregoing, the disease or disorder is selected from the group consisting of post-traumatic stress disorder (PTSD), traumatic brain injury (TBI), and autism. In some embodiments, the disease or disorder is PTSD. In some embodiments, the disease or disorder is autism. In yet other embodiments, the disease or disorder is TBI.

In some embodiments of any of the foregoing embodiments, the plurality, e.g., at least 8, metabolites comprise a metabolite in each of at least 8 of the group of metabolic pathways or in each of the group of metabolic pathways.

In some embodiments of any of the foregoing embodiments, the detection indicates the presence or absence of an alteration in one or more of the group of metabolic pathways, wherein detection of a reduced amount, compared to a normal or control amount, of two or more metabolites in a pathway or an elevated amount, compared to a normal or control amount, of two or more metabolites in a pathway, indicates an alteration in the pathway.

In some embodiments, a determination that at least one of the group of metabolic pathways is altered indicates that the subject is at risk for developing or has the disease or disorder.

In some embodiments, a determination that at least two of the group of metabolic pathways is altered indicates that the subject is at risk for developing or has the disease or disorder. In some embodiments, a determination that at least four of the group of metabolic pathways is altered indicates that the subject is at risk for developing or has the disease or disorder. In some embodiments, a determination that at least 8 of the group of metabolic pathways is altered indicates that the subject is at risk for developing or has the disease or disorder.

In some embodiments of any of the foregoing embodiments, the method further comprises determining that the subject has or is at risk of developing the disease or disorder based on alteration in the group of metabolic pathways.

In some of any of the foregoing embodiments, the subject is a human subject. In some embodiments of any of the foregoing embodiments, the plurality, e.g., at least 8, metabolites comprise metabolites selected from the group consisting of: 2-Octenoylcarnitine, Retinol, L-Tryptophan, Nicotinamide N-oxide, Alanine, L-Tyrosine, 3-Hydroxyanthranilic acid, N-Acetyl-L-aspartic acid, Sarcosine, N-Acetylaspartylglutamic acid, Methylcysteine, AICAR, SM(d18:1/12:0), Oleic acid, Docosahexaenoic acid, Glycocholic acid, Guanosine monophosphate, Cytidine, SM(d18:1/22:0 OH), Xanthine, Indoleacrylic acid, 7-ketocholesterol, 3-Hydroxyhexadecanoylcarnitine, Linoleic acid, Adenosine monophosphate, L-Serine, Pantothenic acid, Arachidonic Acid, PC(26:1), Uracil and combinations thereof. In a further embodiment, the at least 8 metabolites further comprise metabolites selected from the group consisting of: PC(30:2), Hypoxanthine,2-Keto-L-gluconate, Glutaconic acid, 5-HETE, PC(28:2), 3-Hydroxyhexadecenoylcarnitine, Hydroxyproline, Dopamine, Myoinositol, 3-Hydroxylinoleylcarnitine, PC(30:1), LysoPC(24:0), Indole, SM(d18:1/24:0), PC(28:1), L-Threonine, Mevalonic acid, SM(20:0 OH), Purine ring, 3-Hydroxyisobutyroylcarnitine, Dehydroisoandrosterone 3-sulfate, Metanephrine, PC(32:2), PC(34:2), L-Phenylalanine, Phenylpropiolic acid, Methylmalonic acid, Alpha-ketoisocaproic acid, L-Histidine, L-Methionine, PC(18:1(9Z)/18:1(9Z)), 5,6-trans-25-Hydroxyvitamin D3, 2-Methylcitric acid, Taurine, 1-Pyrroline-5-carboxylic acid, L-Proline, PC(18:0/18:2), 7-Methylguanosine, L-Kynurenine, Beta-Alanine, Xanthosine, PE(34:2), Malonylcarnitine, Gluconic acid, L-Glutamine, Pipecolic acid, Cyclic AMP, L-Valine, Cholesterol, SM(d18:1/26:0), L-Lysine, Carbamoylphosphate, Glycerophosphocholine, Adenylosuccinic acid, and combinations thereof.

In some embodiments of any of the foregoing embodiments, the detecting is carried out using one or more of the following: HPLC, TLC, electrochemical analysis, mass spectroscopy, refractive index spectroscopy (RI), Ultra-Violet spectroscopy (UV), fluorescent analysis, gas chromatography (GC), radiochemical analysis, Near-InfraRed spectroscopy (Near-IR), Nuclear Magnetic Resonance spectroscopy (NMR), and Light Scattering analysis (LS).

In some embodiments of any of the foregoing embodiments, the biological sample is selected from the group consisting of cells, cellular organelles, interstitial fluid, blood, blood-derived samples, cerebral spinal fluid, and saliva. In some embodiments, the biological sample is a fluid sample. In some embodiments, the fluid sample is a spinal fluid sample. In some embodiments, the fluid sample is a serum sample. In some embodiments, the fluid sample is a urine sample. In some embodiments of any of the foregoing, the detection is carried out using mass spectroscopy. In some embodiments of any of the foregoing the detection is carried out using a combination of high performance liquid chromatography (HPLC) and mass spectroscopy (MS). In some embodiments, each of the metabolites is measured based on a single run or injection. In any of the foregoing embodiments, the detection includes extracting from the biological sample each of the metabolites from each of the at least 8 metabolic pathways.

In some embodiments, the plurality, e.g., at least 8, metabolites comprise metabolites selected from the group consisting of formate, glycine, serine, catacholamines, serotonin, glutamate, GABA, vitamin B6, thiamine, folate, vitamin B12, glutathione, cysteine and methionine.

In some embodiments of any of the foregoing embodiments, an elevation or reduction in the detected amount of metabolite by at least 1%, 5%, 10%, 15%, 20%, 25%, 30%, 40%, 45%, 50%, 55%, 60%, 65%, 70%, 75%, 80%, 85%, or 90% compared to a control or normal amount indicates an elevation or reduction in the metabolite in the sample.

In some embodiments, the normal or control amount is an amount in a sample from a subject that has not developed the disease or disorder. In some embodiments, the detection comprises converting each of the plurality, e.g., at least 8, metabolites to a non-naturally occurring byproduct and analyzing said byproduct. In a further embodiment, the non-naturally occurring byproduct is a mass fragment or a labeled fragment. In some embodiments, the plurality, e.g., at least 8, metabolites comprise metabolites in at least sixteen (16) metabolic pathways.

The disclosure also provides methods of treating subject having the disease, disorder, or condition. In some embodiments, the methods include carrying out the method of any of the foregoing embodiments, followed by administering, discontinuing, altering, and/or performing therapy or therapeutic intervention on the subject. For example, in some such embodiments, the methods of the foregoing embodiments thereby detect elevated or reduced amounts of one or more of the metabolites compared to a normal or control amounts, and the methods further include performing a therapy on the subject targeted to the disease or disorder. In some embodiments, elevated or reduced amounts of at least 8 metabolites are detected, and/or reduced or elevated levels are detected of metabolites in at least 8 metabolic pathways.

In some embodiments, the methods further include comprises detecting amounts of the at least 8 metabolite in a post-treatment sample from the subject, obtained during or following the treatment. In yet a further embodiment, the method comprises comparing said amounts detected in said post-treatment sample to the amounts detected prior to treatment.

In some embodiments, the provided methods include determining whether a subject has or is at risk of having Post-traumatic Stress Disorder (PTSD). In some embodiments, the methods include detecting a small molecule metabolite profile from a biological sample obtained from the subject; and generating a PTSD metabolomics profile from the small molecule metabolite profile of the subject. In some aspects, the PTSD metabolomics profile includes at least 8 metabolic pathways selected from the group consisting of: a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway; comparing the PTSD metabolomics profile to a normal control PTSD metabolomics profile, wherein when at least one metabolite in the small molecule metabolite profile is aberrantly produced in each of the at least 8 metabolic pathways compared to the control PTSD metabolomics pathway, the subject has or is at risk of having PTSD. In one embodiment, the at least one metabolite comprises at least 2 metabolites in each of the at least 8 metabolic pathways. In a further embodiment, generating the PTSD metabolomics profile from the subject, comprises determining the metabolic activity of each of the following pathways: (i) a phospholipid metabolic pathway; (ii) a fatty acid oxidation and synthesis metabolic pathway; (iii) a purine metabolic pathway; (iv) a bioamine and neurotransmitter metabolic pathway; (v) a microbiome metabolic pathway; (vi) a sphingolipid metabolic pathway; (vii) a cholesterol, cortisol, non-gonadal steroid metabolic pathway; (viii) a pyrimidine metabolic pathway; (ix) a 3- and 4-carbon amino acid metabolic pathway; (x) a branch chain amino acid metabolic pathway; (xi) a tryptophan, kynurenine, serotonin, melatonin metabolic pathway; (xii) a tyrosine and phenylalanine metabolic pathway; (xiii) a SAM, SAH, methionine, cysteine, glutathione metabolic pathway; (xiv) an eicosanoid and resolvin metabolic pathway; (xv) a pentose phosphate, gluconate metabolic pathway; and (xvi) a vitamin A, carotenoid metabolic pathway, comparing the PTSD metabolomics profile from the subject to a control PTSD metabolomics profile comprising the pathways of (i)-(xvi), wherein when at least 8 of the metabolic pathways in (i)-(xvi) have aberrant activity, the subject has or is at risk of having PTSD. In another embodiment, the small molecule metabolite profile comprises metabolites selected from the group consisting of: 2-Octenoylcarnitine, Retinol, L-Tryptophan, Nicotinamide N-oxide, Alanine, L-Tyrosine, 3-Hydroxyanthranilic acid, N-Acetyl-L-aspartic acid, Sarcosine, N-Acetylaspartylglutamic acid, Methylcysteine, AICAR, SM(d18:1/12:0), Oleic acid, Docosahexaenoic acid, Glycocholic acid, Guanosine monophosphate, Cytidine, SM(d18:1/22:0 OH), Xanthine, Indoleacrylic acid, 7-ketocholesterol, 3-Hydroxyhexadecanoylcarnitine, Linoleic acid, Adenosine monophosphate, L-Serine, Pantothenic acid, Arachidonic Acid, PC(26:1), Uracil and any combination thereof. In yet a further embodiment, the small molecule metabolite profile further comprises metabolites selected from the group consisting of: PC(30:2), Hypoxanthine,2-Keto-L-gluconate, Glutaconic acid, 5-HETE, PC(28:2), 3-Hydroxyhexadecenoylcarnitine, Hydroxyproline, Dopamine, Myoinositol, 3-Hydroxylinoleylcarnitine, PC(30:1), LysoPC(24:0), Indole, SM(d18:1/24:0), PC(28:1), L-Threonine, Mevalonic acid, SM(20:0 OH), Purine ring, 3-Hydroxyisobutyroylcarnitine, Dehydroisoandrosterone 3-sulfate, Metanephrine, PC(32:2), PC(34:2), L-Phenylalanine, Phenylpropiolic acid, Methylmalonic acid, Alpha-ketoisocaproic acid, L-Histidine, L-Methionine, PC(18:1(9Z)/18:1(9Z)), 5,6-trans-25-Hydroxyvitamin D3, 2-Methylcitric acid, Taurine, 1-Pyrroline-5-carboxylic acid, L-Proline, PC(18:0/18:2), 7-Methylguanosine, L-Kynurenine, Beta-Alanine, Xanthosine, PE(34:2), Malonylcarnitine, Gluconic acid, L-Glutamine, Pipecolic acid, Cyclic AMP, L-Valine, Cholesterol, SM(d18:1/26:0), L-Lysine, Carbamoylphosphate, Glycerophosphocholine, Adenylosuccinic acid, and any combination thereof.

The disclosure also provides methods of predicting a risk of developing PTSD. In some aspects, the methods are carried out by obtaining a biological sample from a subject; detecting metabolites produced by a pathway selected from the group consisting of: a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway. In some embodiments, the methods include comparing the amount of metabolite to a control value. In some aspects, an aberrant measurement in metabolites from at least 8 of the pathways is indicative of a risk of developing PTSD. In one embodiment, the metabolites are selected from the group consisting of formate, glycine, serine, catacholamines, serotonin, glutamate, GABA, vitamin B6, thiamine, folate, vitamin B12, glutathione, cysteine and methionine. In another embodiment the control corresponds to a normal subject that has not developed PTSD. In another embodiment, the metabolite is converted to a non-naturally occurring by-product that is analyzed. In a further embodiment, the non-naturally occurring by-product is a mass fragment or a labeled fragment.

The disclosure also provides a method of determine if a subject has PTSD comprising obtaining a biological sample from a subject; detecting metabolites produced by a pathway selected from the group consisting of: a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway, and comparing the amount of metabolite to a control value, wherein an aberrant value of any metabolite in 8 or more pathways is indicative of the subject having PTSD.

The disclosure provides methods and compositions for diagnosis of diseases and disorders such as those associated with the cell danger response, inflammation, neuroinflammation, degeneration, and/or neurodegeneration, including neurologic and psychiatric disorders such as, for example, post-traumatic stress disorder (PTSD) and Traumatic Brain Injury (TBI), by analyzing metabolites found in easily obtained biospecimens (e.g., blood and urine). Among the provided methods are those that allow clinicians to stratify military recruits and patients according to the future risk of PTSD. In some embodiments, the methods use high performance liquid chromatography (HPLC) chromatography, tandem. Mass Spectrometry (LC-MS/MS), and analytical statistical techniques. While several hundred analytes are in some embodiments measured, in practice, 30 or fewer, e.g., 30, 25, 20, 16, 15, or fewer, analytes and/or pathways may be sufficient for diagnostic and prognostic purposes. Analysis of these analytes may be performed with various techniques, including chromatography and mass spectrometry methods and combinations thereof, including HPLC and/or Mass Spectrometry.

In some embodiments, the assessment and/or detection and/or determining involves statistical analyses, e.g., based on the amounts detected and/or control amounts.

Also provided are compositions and articles of manufacture for carrying out the methods, including kits containing positive control compounds for a 1 or some of the metabolites and/or pathways detected and/or measured, in any of the foregoing embodiments.

In some embodiments, the methods and compositions of the disclosure can be used to diagnose psychiatric and/or neurological disorders, including but not limited to pervasive developmental disorder not otherwise specified, non-verbal learning disabilities, autism and autism spectrum disorders, attention deficit hyperactivity disorder (ADHD), anxiety disorders, Post-traumatic stress disorders, traumatic brain injury (TBI), social phobia, generalized anxiety disorder, social deficit disorders, schizotypal personality disorder, schizoid personality disorder, schizophrenia, cognitive deficit disorders, dementia, Alzheimer's and other memory deficit disorders.

### DESCRIPTION OF DRAWINGS

FIG. 1 shows a plot of metabolomics diagnosis of post-traumatic stress disorder (PTSD).

FIG. 2 shows a plot of metabolic prediction of PTSD risk.

FIG. 3 shows a plot of metabolomics diagnosis of TBI.

FIG. 4 shows a rank-order of metabolites used to diagnose PTSD.

FIG. 5 depicts an overview of a process for metabolite analysis used in the methods of the disclosure.

FIG. 6 shows a general study design of the disclosure.

FIG. 7 shows a chart of metabolomics risk stratification for PTSD.

FIG. 8 shows diagrams of PTSD pathway analysis in smokers and non-smokers.

FIG. 9 shows diagrams of PTSD and TBI analysis.

FIG. 10 shows pathways enriched in predeployment marines who later develop PTSD.

FIG. 11 shows a brief summary of signatures related to PTSD, TBI and risk of PTSD.

FIG. 12 shows metabolic pathways, alterations in which were observed to be shared by MIA and Fragile X mouse models for autism. 11 of the 18 pathways, alterations of which characterized the maternal immune activation (MIA), and of the 20 pathways alterations of which characterized the Fragile X model were shared. These common 11 pathways were: purine metabolism, microbiome metabolism, phospholipid metabolism, sphingolipid metabolism, cholesterol metabolism, bile acid metabolism, glycolysis, Krebs cycle, Vitamin B3 (Niacin, NAD+) metabolism, pyrimidine metabolism, and S-adenosylmethionine (SAM)/S-adenosylhomocysteine (SAH)/glutathione (GSH) metabolism.

FIG. 13 shows cytoscape visualization of metabolic pathways altered by antipurinergic therapy in the Fragile X mouse model. Twenty-six of the 60 biochemical pathways interrogated in the metabolomic analysis are illustrated. See Tables 5 and 6B for complete listing of pathways and discriminating metabolites, respectively. The fractional contribution of each of the top 20 pathways altered by suramin treatment is indicated as a percentage of the total variable importance in projection (VIP) score in the black circles. Purine metabolism accounted for 20% of the variance, followed by fatty acid oxidation (12%), eicosanoids (11%), gangliosides (10%), phospholipids (9%), and 15 other biochemical pathways as indicated.

FIG. 14A and B shows results of a study demonstrating correction, by antipurinergic therapy, of widespread metabolomic alterations in the Fragile X Mouse Model compared with normal control animals. (A) Multivariate Analysis of Metabotypes Associated with Suramin (KO-Suramin) and Saline Treatment (KO-Saline) Compared to FVB-Controls Treated with Saline. 673 plasma metabolites from 60 biochemical pathways were measured by liquid chromatography tandem mass spectrometry (LC-MS/MS) and analyzed by partial least squares discriminant analysis (PLSDA). The 3 top multivariate components were then plotted on x, y, and z-axes, respectively. Suramin treatment shifted metabolism in the direction of wild-type controls. N=9-11 per group. (B) Metabolites and Pathways Associated with Suramin Treatment in the Fragile X Model. The top 30 most discriminating metabolites are shown, with their biochemical pathways ranked by variable importance in projection (VIP) scores. See Table 6B for a complete list of the top 58 discriminating metabolites. VIP scores ≧1.5 were deemed statistically significant.

FIG. 15A-D shows metabolomic analysis of APT treatment in MIA mouse model. (A) APT rescues widespread metabolic abnormalities. Plasma samples were collected 2 days after a single dose of suramin (20 mg kg⁻¹ i.p.) or saline (5 μl g⁻¹ i.p.). This analysis shows that a single dose of suramin (PIC-Sur) drives the metabolism of MIA animals (PIC-Sal) strongly in the direction of controls (Sal-Sal). Metabolomic profiles in this study were assessed by detecting and quantifying 478 metabolites from 44 biochemical pathways, measured with LC-MS/MS. N=6, 6.5-month-old males per group. (B) Metabolic memory preserved metabolic rescue by APT. The analysis showed that 5 weeks after a single dose of suramin (PIC-Sur W/O) the metabolism of treated animals had drifted back toward that of untreated, MIA animals (PIC-Sal; N=6 males per group). (C) Hierarchical clustering of suramin-treated and suramin-washout metabotypes. This analysis illustrated the metabolic similarity between control (Sal-Sal) animals and MIA animals treated with one dose of suramin (PIC-Sur), as compared with metabolic profiles of saline-treated MIA animals (PIC-Sal) and ASD-like animals tested 5 weeks after suramin washout (PIC-Sur W/O). The numbers listed along the x axis are animal ID numbers. (D) Rank Order of metabolites disturbed in the MIA model. Multivariate analysis across the four treatment groups (PIC-Sal=MIA; PIC-Sur=acute suramin treatment; PIC-Sur w/o=5 weeks post-suramin washout; Sal-Sal=Controls). Biochemical pathway assignments are listed on the left. Relative magnitudes of each metabolite disturbance are listed on the right as high, intermediate and low. Variable importance in projection (VIP) scores were multivariate statistics that reflected the impacts of the respective metabolite on the partial least squares discriminant analysis model. VIP scores above 1.5 were deemed significant.

### DETAILED DESCRIPTION

As used herein and in the appended claims, the singular forms “a,” “and,” and “the” include plural referents unless the context clearly dictates otherwise. Thus, for example, reference to “a sample” includes a plurality of such samples and reference to “the subject” includes reference to one or more subjects, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Although methods and materials similar or equivalent to those described herein can be used in the practice of the disclosed methods and compositions, the exemplary methods, devices and materials are described herein.

Unless defined otherwise, all technical and scientific terms used herein have the same meaning as commonly understood to one of ordinary skill in the art to which this disclosure belongs.

All publications mentioned herein are incorporated by reference in full for the purpose of describing and disclosing the methodologies that might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Molecular biology techniques for uncovering the biochemical processes underlying disease have been centered on the genome, which consists of the genes that make up DNA, which is transcribed into RNA and then translated to proteins, which then function in metabolic pathways to generate the small molecules of the human metabolome. While genomics (study of the DNA-level biochemistry), transcript profiling (study of the RNA-level biochemistry), and proteomics (study of the protein-level biochemistry) are useful for identification of disease pathways, these methods are complicated by the fact that there exist over tens of thousands of genes, hundreds of thousands of RNA transcripts and up to a million proteins in human cells. However, it is estimated that there may be as few as 2,500 small molecules in the human metabolome.

Metabolomics is the study of the small molecules, or metabolites, contained in a cell, tissue or organ (including fluids) and involved in primary and intermediary metabolism. Thus, metabolomics in some embodiments reflects a direct observation of the status of cellular physiology, and may thus be predictive of disease in a given organism. Subtle biochemical changes (including the presence of selected metabolites) can be reflective of a given disease, disorder, condition, or physiological state, or class thereof. The accurate mapping of such changes to known metabolic pathways can permit researchers to build, e.g., a biochemical hypothesis for a disease. Based on this hypothesis, the enzymes and proteins critical to or characteristic of the disease can be uncovered such that disease targets may be identified for treatment with targeted pharmaceutical compounds or other therapy. Thus, in some aspects, metabolomic technologies can offer advantages compared with other approaches such as genomics, transcript profiling, and/or proteomics. With metabolomics, metabolites, and their role in the metabolism may be readily identified. In this context, the identification of disease targets may be expedited with greater accuracy relative to other known methods.

“Acute stress disorder” is an anxiety disorder that involves a reaction following exposure to a traumatic event or stressor (e.g., a serious injury to oneself, witnessing an act of violence, hearing about something horrible that has happened to someone one is close to). While similar to PTSD, the duration of symptoms of acute stress disorder is shorter than that for PTSD. In some embodiments, a clinical diagnosis of acute stress disorder indicates that the symptoms may be present for two days to four weeks.

The term “biological sample” refers to any sample obtained from a subject. Exemplary biological samples include, but are not limited to, fluid samples, such as urine, feces, blood, blood components, such as serum, saliva, sweat, and/or spinal and brain fluid, organ and tissue samples.

The term “metabolic pathway” refers to a series or set of anabolic or catabolic biochemical reactions in a living organism (“metabolic reactions”) that convert (transmuting) one chemical species into another.

The term “metabolite” refers to any substance produced by or transmutated in a metabolic reaction. A “metabolite” is considered to be in or belong to a particular metabolic pathway if it is a precursor, product, and/or intermediate of the pathway and/or if the pathway's precursor or product is readily traceable to the metabolite. Such a metabolite can be an organic compound that is a starting material, an intermediate in, or an end product of the metabolic pathway. Metabolites include molecules that during metabolism are used to construct more complex molecules and/or that are broken down into simpler ones. The term includes end products and intermediate metabolites

In some embodiments, the presence and/or amount(s)/level(s) of specific metabolite(s) in a given metabolic pathway (e.g. products or intermediates of the pathway), and/or collections of such metabolites, are detected or measured, for example, by mass spectrometry and/or chromatography. In some embodiments, such detected amounts are compared to normal or control amounts. In some embodiments, the detected amounts are used to assess or detect alterations in the metabolic pathway, which in some aspects is informative for diagnosis and/or prediction of disease(s) or condition(s).

The term “metabolome” refers to the collection of metabolites present in an organism. The human metabolome encompasses native small molecules (natively biosynthesizeable, non-polymeric compounds) that are participants in general metabolic reactions and that are part of the maintenance, growth and function of a cell or tissue.

The terms “patient” and “subject” encompass both human and non-human organisms, including non-human mammals. The term “subject” includes patients and also includes other persons and organisms, e.g., animals. For example, the term encompasses subjects diagnosed or analyzed by the methods of the disclosure or from which biological samples are derived.

Post-Traumatic Stress Disorder (PTSD) is a disorder that can develop after exposure to one or more traumatic event or ordeal, such as one in which grave physical harm occurred or was threatened to oneself or others, sexual assault, warfare, serious injury, or threats of imminent death, that result in feelings of intense fear, horror, and/or powerlessness.

Traumatic events that may trigger PTSD include violent personal assaults, natural or human-caused disasters, accidents, or military combat, all of which can involve traumatic brain injury (TBI). PTSD was described in veterans of the American Civil War, and was called “shell shock,” “combat neurosis,” and “operational fatigue.” PTSD symptoms can be grouped into three categories: (1) re-experiencing symptoms; (2) avoidance symptoms; and (3) hyperarousal symptoms. Exemplary re-experience symptoms include flashbacks (e.g., reliving the trauma over and over, including physical symptoms like a racing heart or sweating), bad dreams, and frightening thoughts. Re-experiencing symptoms may cause problems in a person's everyday routine. They can start from the person's own thoughts and feelings. Words, objects, or situations that are reminders of the event can also trigger re-experiencing. Symptoms of avoidance include staying away from places, events, or objects that are reminders of the experience; feeling emotionally numb; feeling strong guilt, depression, or worry; losing interest in activities that were enjoyable in the past; and having trouble remembering the dangerous event. Things that remind a person of the traumatic event can trigger avoidance symptoms. These symptoms may cause a person to change his or her personal routine. For example, after a bad car accident, a person who usually drives may avoid driving or riding in a car. Hyperarousal symptoms include being easily startled, feeling tense or “on edge”, having difficulty sleeping, and/or having angry outbursts. Hyperarousal symptoms are usually constant, instead of being triggered by things that remind one of the traumatic event. They can make the person feel stressed and angry. These symptoms may make it hard to do daily tasks, such as sleeping, eating, or concentrating. Therefore, generally, PTSD symptoms can include nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, stress, and anxiety. The symptoms may last for a month, for three months, or for longer periods of time.

The term “small molecules” includes organic and inorganic molecules, such as those present in a biological sample obtained from a patient or subject. Examples of small molecules include sugars, fatty acids, amino acids, nucleotides, intermediates formed during cellular processes, and other small molecules found within a cell. In some embodiments, the small molecules are metabolites.

The term “small molecule metabolite profile” refers to the composition, amounts, and/or identity, of small molecule metabolites present in a biological sample, a cell, tissue, organ, or organism. The small molecule metabolite profile provides information related to the metabolism or metabolic pathways that are active in a cell, tissue or organism. Thus, the small molecule metabolite profile provides data for developing a “metabolomic profile” (also referred to as “metabolic profile”) of active or inactive metabolic pathways in a cell, tissue, or subject. The small molecule metabolite profile includes, e.g., the quantity and/or type of small molecules present. A “small molecule metabolite profile,” can be obtained using a single measurement technique (e.g., HPLC) or a combination of techniques (e.g., HPLC and mass spectrometry). The type of small molecule to be measured will determine the technique to be used and can be readily determined by one of skill in the art.

“Traumatic brain injury (TBI)” refers to damage to the brain as the result of an injury. TBI usually results from a violent blow or jolt to the head that causes the brain to collide with the inside of the skull. An object penetrating the skull, such as a bullet or shattered piece of skull, can also cause TBI. Depending on the severity of the blow or jolt to the head, TBI can be a mild TBI or moderate to severe TBI. Mild TBI may cause temporary dysfunction of brain cells. More serious TBI can result in bruising, torn tissues, bleeding and other physical damage to the brain that can result in long-term complications. The signs and symptoms of mild TBI may include: confusion or disorientation, memory or concentration problems, headache, dizziness or loss of balance, nausea or vomiting, sensory problems, such as blurred vision, ringing in the ears or a bad taste in the mouth, sensitivity to light or sound, mood changes or mood swings, feeling depressed or anxious, fatigue or drowsiness, difficulty sleeping, or sleeping more than usual. Moderate to severe TBI can include any of the signs and symptoms of mild injury, as well as the following symptoms that may appear within the first hours to days after a head injury: profound confusion, agitation, hyperexcitability, combativeness or other unusual behavior, slurred speech, inability to awaken from sleep, weakness or numbness in the extremities, loss of coordination, persistent headache or headache that worsens, convulsions or seizures. Symptoms of TBI also include cognitive or memory impairments and motor deficits. TBI may cause negative effects such as emotional, social, or behavioral problems, changes in personality, emotional instability, depression, anxiety, hypomania, mania, apathy, irritability, problems with social judgment, and impaired conversational skills. TBI appears to predispose survivors to psychiatric disorders including obsessive compulsive disorder, substance abuse, dysthymia, clinical depression, bipolar disorder, and anxiety disorders. In patients who have depression after TBI, suicidal ideation is common; the suicide rate among these patients increase 2- to 3-fold. Social and behavioral effects that can follow TBI include disinhibition, inability to control anger, impulsiveness, and lack of initiative.

A “metabolomic profile” is a profile of pathway activity associated with the small molecule metabolites. The activity of the pathways is an indication of metabolic health. For example, one or more small molecule metabolites can be measured in a specific pathway, the small molecule metabolites can include intermediates as well as the end product. The metabolomics profile identifies the pathway's “activity”. If the pathway produced a normal amount of the metabolite, then the pathway is normal, however, if the pathway produces excessive or reduced amounts then the pathway has aberrant activity. Typically a disease state (or risk thereof) is identified by a plurality of aberrant pathways in a metabolomics profile. The pathway can be identified numerically, by color, by code or other symbols as being aberrant or normal. In the human body, a vast number of metabolic pathways are well characterized including substrates, intermediates, products, enzymes, genes and the like. One of skill in the art can readily identify the pathways and their metabolites and interconnectedness with other pathways. For example, Sigma-Aldrich has an on-line, interactive metabolic pathway for numerous species including humans (see, e.g., [http://]www[.]sigmaaldrich.com/technical-documents/articles/biology/interactive-metabolic-pathways-map.html) (note that the foregoing has been modified with brackets to eliminate an active hyperlink). For particular disease states, the disclosure provides certain metabolomics profiles that are useful for diagnosis (e.g., a “PTSD metabolomics profile”, an “autism spectrum disorder (ASD) metabolomics profile”, a “traumatic brain injury (TBI) metabolomics profile”, and the like).

A small molecule metabolite profile and metabolomic profile can be obtained for normal control (e.g., a “control small molecule metabolite profile” or “control metabolomic profile”) and would include an inventory of small molecules or metabolomic pathways that are active in similar cells, tissue or sample from a population of subject that are considered “normal” or “healthy” (e.g., lack any disease or disorder traits or phenotypic characteristics relative to a specific disease or disorder being examined). For example, where PTSD is to be determined or the risk of PTSD is to be determined a “control small molecule metabolite profile” or “control metabolomic profile” would include the inventory and amounts of small molecules present (or metabolic pathways active) in, e.g., 70%, 80%, or 90%, but typically greater than 95% of a population that does not have any symptoms of PTSD.

In some embodiments, small molecule metabolite profile(s) or metabolomic profile(s) from a test subject or patient is/are compared to that/those of a control small molecule or control metabolomic profile. In some embodiments, detected amounts of metabolites are compared to normal or control amounts, such as amounts detected performing similar methods on a normal or control sample. A normal or control sample in some aspects is one obtained from a subject who does not have, or is known not to have developed, e.g., subsequent to obtaining the sample, the disease or disorder being assessed, or having a relatively low risk for the same. Such comparisons can be made by individuals, e.g., visually, or can be made using software designed to make such comparisons, e.g., a software program may provide a secondary output which provides useful information to a user. For example, a software program can be used to confirm a profile or can be used to provide a readout when a comparison between profiles is not possible with a “naked eye”. The selection of an appropriate software program, e.g., a pattern recognition software program, is within the ordinary skill of the art. An example of such a program is Pirouette® by InfoMetrix®.

Also as used herein, the term “test metabolite” is intended to indicate a substance the concentration of which in a biological sample is to be measured; the test metabolite is a substance that is a by-product of or corresponds to a specific end product or intermediate of metabolism.

The collection of metabolomic data, including small molecule metabolite profiles and metabolic profiles, can be through, for example, a single technique or a combination of techniques for separating and/or identifying small molecules known in the art. Small molecule metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection.

Chromatography, such as gas chromatography (GC) and high pressure liquid chromatography (HPLC), in some embodiments is used in the process of detecting and quantifying (e.g., detecting an amount of) one or more metabolites.

For example, in some embodiments, High Performance Liquid Chromatography (HPLC) is used in a method for identifying and/or separating a small molecule metabolite. HPLC columns equipped with coulometric array technology can be used to analyze the samples, separate the compounds, and/or create a small molecule metabolite profiles of the samples. HPLC columns are known and have been used in serum, urine and tissue analysis and are suitable for small molecule analysis (Beal et al., J Neurochem., 55:1327-1339, 1990; Matson et al., Life Sci., 41:905-908, 1987; Matson et al., Basic, Clinical and Therapeutic Aspects of Alzheimer's and Parkinson's Diseases, vol II, pp. 513-516, Plenum, N.Y. 1990; LeWitt et al., Neurology, 42:2111-2117, 1992; Ogawa et al., Neurology, 42:1702-1706, 1992; Beal et al., J. Neurol. Sci., 108:80-87, 1992; Matson et al., Clin. Chem., 30:1477-1488, 1984; Milbury et al., Coulometric Electrode Array Detectors for HPLC, pp. 125-141, VSP International Science Publication; Acworth et al., Am. Lab, 28:33-38, 1996).

In GC, the sample to be analyzed is introduced via a syringe into a narrow bore (capillary) column which sits in an oven. The column, which typically contains a liquid adsorbed onto an inert surface, is flushed with a carrier gas such as helium or nitrogen. In a properly set up GC system, a mixture of substances introduced into the carrier gas is volatilized, and the individual components of the mixture migrate through the column at different speeds. Detection takes place at the end of the heated column and is generally a destructive process. Very often the substance to be analyzed is “derivatized” to make it volatile or change its chromatographic characteristics. In contrast, for HPLC a liquid under high pressure is used to flush the column rather than a gas. Typically, the column operates at room or slightly above room temperature.

In some embodiments, Mass Spectroscopy (MS) Detectors are used in the identification and/or quantification of the metabolites. The sample, fraction thereof, compound, and/or molecule generally is ionized and passed through a mass analyzer where the ion current is detected. There are various methods for ionization. Examples of these methods of ionization include, but are not limited to, electron impact (EI) where an electric current or beam created under high electric potential is used to ionize the sample migrating off the column; chemical ionization utilizes ionized gas to remove electrons from the compounds eluting from the column; and fast atom bombardment where Xenon atoms are propelled at high speed in order to ionize the eluents from the column.

Gas chromatography/mass spectrometry (GC/MS) is a combination of two technologies. GC physically separates (chromatographs or purifies) the compound, and MS fragments it so that a fingerprint of the chemical can be obtained. Although sample preparation is extensive, using the methods together can improve accuracy, sensitivity, and/or specificity. The combination is sensitive (i.e., can detect low levels) and specific. Furthermore, assay sensitivity can be enhanced by treating the test substance with reagents.

Liquid chromatography/mass spectrometry (LC/MS) is a combination of liquid chromatography methods and mass spectrometry methods. Liquid chromatography such as HPLC, when coupled with MS, provides improved accuracy, specificity, and/or sensitivity, for example, in detection of substances that are difficult to volatilize.

In some embodiments, Pyrolysis Mass Spectrometry can be used to identify and/or quantify small molecule metabolites. Pyrolysis is the thermal degradation of complex material in an inert atmosphere or vacuum. It causes molecules to cleave at their weakest points to produce smaller, volatile fragments called pyrolysate. Curie-point pyrolysis is a particularly reproducible and straightforward version of the technique, in which the sample, dried onto an appropriate metal is rapidly heated to the Curie-point of the metal. A mass spectrometer can then be used to separate the components of the pyrolysate on the basis of their mass-to-charge ratio to produce a pyrolysis mass spectrum (Meuzelaar et al. 1982) which can then be used as a “chemical profile” or fingerprint of the complex material analyzed. The combined technique is known as pyrolysis mass spectrometry (PyMS).

In another embodiment, Nuclear Magnetic Resonance (NMR) can be used to identify and/or quantify small molecule metabolites. Certain atoms with odd-numbered masses, including H and ¹³C, spin about an axis in a random fashion. When they are placed between poles of a strong magnet, the spins are aligned either parallel or anti-parallel to the magnetic field, with parallel orientation favored since it is slightly lower energy. The nuclei are then irradiated with electromagnetic radiation which is absorbed and places the parallel nuclei into a higher energy state where they become in resonance with radiation.

In yet another embodiment, Refractive Index (RI) can be used to identify and/or quantify small molecule metabolites. In this method, detectors measure the ability of samples to bend or refract light. Each small molecule metabolite has its own refractive index. For most RI detectors, light proceeds through a bi-modular flow to a photodetector. One channel of the flow-cell directs the mobile phase passing through the column while the other directs only the other directs only the mobile phase. Detection occurs when the light is bent due to samples eluting from the column, and is read as a disparity between the two channels. Laser based RI detectors have also become available.

In another embodiment, Ultra-Violet (UV) Detectors can be used to identify and/or quantify small molecule metabolites. In this method, detectors measure the ability of a sample to absorb light. This could be accomplished at a fixed wavelength usually 254 nm, or at variable wavelengths where one wavelength is measured at a time and a wide range is covered, alternatively Diode Array are capable of measuring a spectrum of wavelengths simultaneously. Sensitivity is in the 10⁻⁸ to 10⁻⁹ gm/ml range. Laser based absorbance or Fourier Transform methods have also been developed.

In another embodiment, Fluorescent Detectors can be used to identify and/or quantify small molecule metabolites. This method measure the ability of a compound to absorb then re-emit light at given wavelengths. Each compound has a characteristic fluorescence. The excitation source passes through the flow-cell to a photodetector while a monochromator measures the emission wavelengths. Sensitivity is in the 10⁻⁹ to 10⁻¹¹ gm/ml. Laser based fluorescence detectors are also available.

In yet another embodiment, Radiochemical Detection methods can be used to identify and/or quantify small molecule metabolites. This method involves the use of radiolabeled material, for example, tritium or carbon 14. It operates by detection of fluorescence associated with beta-particle ionization, and it is most popular in metabolite research. The detector types include homogeneous detection where the addition of scintillation fluid to column effluent causes fluorescence, or heterogeneous detection where lithium silicate and fluorescence by caused by beta-particle emission interact with the detector cell. Sensitivity is 10⁻⁹ to 10⁻¹⁰ gm/ml.

Electrochemical Detection methods can be used to identify and/or quantify small molecule metabolites. Detectors measure compounds that undergo oxidation or reduction reactions. Usually accomplished by measuring gains or loss of electrons from migration samples as they pass between electrodes at a given difference in electrical potential. Sensitivity of 10⁻¹² to 10⁻¹³ gms/ml.

Light Scattering (LS) Detector methods can be used to identify and/or quantify small molecule metabolites. This method involves a source which emits a parallel beam of light. The beam of light strikes particles in solution, and some light is then reflected, absorbed, transmitted, or scattered. Two forms of LS detection may be used to measure transmission and scattering.

Nephelometry, defined as the measurement of light scattered by a particular solution. This method enables the detection of the portion of light scattered at a multitude of angles. The sensitivity depends on the absence of background light or scatter since the detection occurs at a black or null background. Turbidimetry, defined as the measure of the reduction of light transmitted due to particles in solution. It measures the light scatter as a decrease in the light that is transmitted through particulate solution. Therefore, it quantifies the residual light transmitted. Sensitivity of this method depends on the sensitivity of the machine employed, which can range from a simple spectrophotometer to a sophisticated discrete analyzer. Thus, the measurement of a decrease in transmitted light from a large signal of transmitted light is limited to the photometric accuracy and limitations of the instrument employed.

Near Infrared scattering detectors operate by scanning compounds in a spectrum from 700-1100 nm. Stretching and bending vibrations of particular chemical bonds in each molecule are detected at certain wavelengths. This method offers several advantages; speed, simplicity of preparation of sample, multiple analyses from single spectrum and nonconsumption of the sample.

Fourier Transform Infrared Spectroscopy (FT-IR) can be used to identify and/or quantify small molecule metabolites. This method measures dominantly vibrations of functional groups and highly polar bonds. The generated fingerprints are made up of the vibrational features of all the sample components (Griffiths 1986). FT-IR spectrometers record the interaction of IR radiation with experimental samples, measuring the frequencies at which the sample absorbs the radiation and the intensities of the absorptions. Determining these frequencies allows identification of the samples chemical makeup, since chemical functional groups are known to absorb light at specific frequencies. Both quantitative and qualitative analysis are possible using the FT-IR detection method.

Dispersive Raman Spectroscopy is a vibrational signature of a molecule or complex system. The origin of dispersive raman spectroscopy lies in the inelastic collisions between the molecules composing say the liquid and photons, which are the particles of light composing a light beam. The collision between the molecules and the photons leads to an exchange of energy with consequent change in energy and hence wavelength of the photon.

Immunoassay methods are based on an antibody-antigen reaction, small amounts of the drug or metabolite(s) can be detected. Antibodies specific to a particular drug are produced by injecting laboratory animals with the drug or human metabolite. These antibodies are then tagged with markers such as an enzyme (enzyme immunoassay, EIA), a radio isotope (radioimmunoassay, RIA) or a fluorescence (fluorescence polarization immunoassay, FPIA) label. Reagents containing these labeled antibodies can then be introduced into urine samples, and if the specific drug or metabolite against which the antibody was made is present, a reaction will occur.

A biological sample obtained from a subject can be prepared for use in one or more of the foregoing identification/detection methods. The biological sample, can be divided for multiple parallel measurements and/or can be enriched for a particularly type of small molecule metabolite(s). For example, different fractionation procedures can be used to enrich the fractions for small molecules. For example, small molecules obtained can be passed over several fractionation columns. The fractionation columns will employ a variety of detectors used in tandem or parallel to generate the small molecule metabolite profile.

For example, to generate a small molecule metabolite profile of water soluble molecules, the biological sample will be fractionated on HPLC columns with a water soluble array. The water soluble small molecule metabolites can then be detected using fluorescence or UV detectors to generate the small molecule metabolite profiles. For detecting non water soluble molecules, hydrophobic columns can also be used to generate small molecule metabolite profiles. In addition, gas chromatography combined with mass spectroscopy, liquid chromatography combined with mass spectroscopy, MALDI combined with mass spectroscopy, ion spray spectroscopy combined with mass spectroscopy, capillary electrophoresis, NMR and IR detection are among the many other combinations of separation and detection tools can be used to generate small molecule metabolite profiles.

Provided are methods to diagnose and/or provide predictive and/or risk information about certain neurologic or psychiatric disorders, such as post-traumatic stress disorder (PTSD), autism spectrum disorder (ASD) and Traumatic Brain Injury (TBI) by analyzing metabolites found in easily obtained biospecimens (e.g., blood, urine). In one embodiment, the methods of the disclosure allows clinicians to stratify military recruits and patients according to the risk of PTSD or the occurrence of PTSD. In one embodiment, the methods use high performance liquid chromatography (HPLC) chromatography, tandem Mass Spectrometry (LC-MS/MS), and analytical statistical techniques to identify and analyze metabolomic profiles.

The methods of the disclosure can utilize the measurement of a thousand or more metabolites (e.g., up to 2500 or more) or fewer than 2500 (e.g., 15-30, 30-60, 60-100, 100-200, 200-500, 500-1000, 1000-1500, 1500-2000, 2000-2500 and any number there between 15 and 2500). While several hundred small molecule metabolites can be measured, in practice 30 or fewer small molecule metabolites may be sufficient for diagnostic and prognostic purposes. Furthermore, the small molecule metabolites being measured can include more than one metabolite from a particular metabolic pathway. Thus, for example, 30 or fewer small molecule metabolites may be representative of 15 or fewer metabolic pathways (e.g., more than one metabolite is from the same catabolic or anabolic pathway). Analysis of these metabolites may be performed using HPLC and Mass Spectrometry or with techniques other than HPLC and/or Mass Spectrometry.

For example, small molecule metabolites are collected and subjected to chemical extraction. Internal isotopically labeled standards can be added to the sample and injected into an HPLC-Mass Spectrometer. Small molecule metabolites are separated and then measured via mass spectrometry. Subjects having or at risk of having PTSD (or other disease or disorder to be analyzed) have a distinct set of metabolites (e.g., a “PTSD small molecule metabolite profile”) that are indicative of a PTSD metabolomic profile that distinguish them from healthy controls.

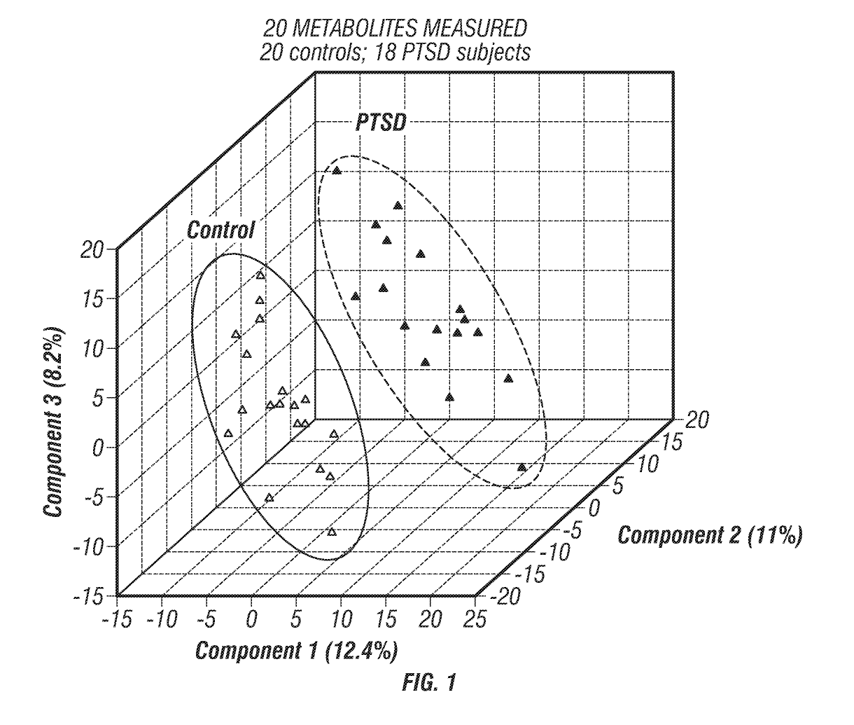

In some embodiments, the small molecule metabolites are collected, processed to non-naturally occurring analytes (e.g., mass fragments), the analytes processed to determine their identities and the data plotted in 2D or 3D coordinates and compared to a control small molecule metabolite profile or a control metabolomics profile, which can be plotted on the same coordinate system (e.g., a mass spectroscopy plot, an HPLC plot or the like) (see, e.g., FIG. 1-3). This plot can then be output to a user or medical technician for analysis.

For example, the method of the disclosure includes obtaining a small molecule metabolite profile from a test subject, identifying small molecule analytes that are over produced or under produced (including presence and absence) generating a metabolomics profile which is indicative of the activity of the various metabolic pathways associated with the small molecule metabolites and comparing metabolomics profiles of the test subject/patients to a standard, normal control metabolomics profile. In one embodiment, an over or under production of a metabolite compared to a control by at least 2 standard deviations is indicative of an aberrant metabolic pathway. In another embodiment, a difference in the amount of metabolite by 10% or more (e.g., 10%-100% or more) compared to a control value is indicative of an aberrant metabolic pathway. The method thus involves identifying the small molecules which are present in aberrant amounts in the test small molecule metabolite profile. The small molecules present in aberrant amounts are indicative of a diseased or dysfunctional metabolic pathway.

An “aberrant amount” includes any level, amount, or concentration of a small molecule metabolite, which is different from the level of the small molecule of a standard sample by at least 1 standard deviation (typically 2 standard deviations is used). The aberrant amount can be higher or lower than the control amount.

The method of the disclosure include measuring a plurality of pathways and metabolites. Table 1, provides an exemplary list of 63 such pathways and an exemplary number of metabolites that can be measure in each pathway.

TABLE 1

| Pathway | Metabolites |
|---|---|
| 1-Carbon, Folate, Formate, Glycine Metabolism | 7 |
| Amino Acid Metabolism not otherwise covered | 6 |
| Antibiotics, Pesticides, and Xenobiotic Metabolism | 10 |
| Bile Salt Metabolism | 8 |
| Bioamines and Neurotransmitter Metabolism | 14 |
| Biopterin, Neopterin, Molybdopterin Metabolism | 1 |
| Biotin (Vitamin B7) Metabolism | 1 |
| Branch Chain Amino Acid Metabolism | 13 |
| Cardiolipin Metabolism | 12 |
| Cholesterol, Cortisol, Non-Gonadal Steroid Metabolism | 29 |
| Drugs of Abuse | 24 |
| Eicosanoid and Resolvin Metabolism | 35 |
| Endocannabinoid Metabolism | 2 |
| Fatty Acid Oxidation and Synthesis | 40 |
| Food Sources, Additives, Preservatives, Colorings, and | 4 |
| Dyes | |
| GABA, Glutamate, Arginine, Ornithine, Proline | 7 |
| Metabolism | |
| Gamma-Glutamyl and other Dipeptides | 6 |
| Glycolipid Metabolism | 11 |
| Glycolysis, Gluconeogenesis Metabolism | 19 |
| Gonadal Steroids | 7 |
| Heme and Porphyrin Metabolism | 5 |
| Histidine, Histamine Metabolism | 5 |
| Isoleucine, Valine, Threonine, or Methionine Metabolism | 5 |
| Ketone Body Metabolism | 2 |
| Krebs Cycle | 18 |
| Lysine Metabolism | 3 |
| Microbiome Metabolism | 36 |
| Neuropeptide Hormones | 1 |
| Nitric Oxide, Superoxide, Peroxide Metabolism | 7 |
| Amino-Sugar and Galactose Metabolism | 10 |
| OTC and Prescription Pharmaceutical Metabolism | 98 |
| Oxalate, Glyoxylate Metabolism | 3 |
| Pentose Phosphate, Gluconate Metabolism | 11 |
| Phosphate and Pyrophosphate Metabolism | 1 |
| Phospholipid Metabolism | 133 |
| Phytanic, Branch, Odd Chain Fatty Acid Metabolism | 2 |
| Phytonutrients, Bioactive Botanical Metabolites | 4 |
| Plasmalogen Metabolism | 3 |
| Plastics, Phthalates, Parabens, and Personal Care Products | 2 |
| Polyamine Metabolism | 9 |
| Purine Metabolism | 49 |
| Pyrimidine Metabolism | 36 |
| SAM, SAH, Methionine, Cysteine, Glutathione Metabolism | 24 |
| Sphingolipid Metabolism | 79 |
| Taurine, Hypotaurine Metabolism | 2 |
| Thyroxine Metabolism | 1 |
| Triacylglycerol Metabolism | 1 |
| Tryptophan, Kynurenine, Serotonin, Melatonin Metabolism | 11 |
| Tyrosine and Phenylalanine Metabolism | 4 |
| Ubiquinone Metabolism | 4 |
| Urea Cycle | 4 |
| Very Long Chain Fatty Acid Oxidation | 3 |
| Vitamin A (Retinol), Carotenoid Metabolism | 3 |
| Vitamin B1 (Thiamine) Metabolism | 4 |
| Vitamin B12 (Cobalamin) Metabolism | 4 |
| Vitamin B2 (Riboflavin) Metabolism | 4 |
| Vitamin B3 (Niacin, NAD+) Metabolism | 8 |
| Vitamin B5 (Pantothenate, CoA) Metabolism | 1 |
| Vitamin B6 (Pyridoxine) Metabolism | 6 |
| Vitamin C (Ascorbate) Metabolism | 2 |
| Vitamin D (Calciferol) Metabolism | 2 |
| Vitamin E (Tocopherol) Metabolism | 1 |
| Vitamin K (Menaquinone) Metabolism | 1 |
| Subtotal | 868 |
| TOTAL Pathways and Chemical Sources | 63 |

Various statistical methods can be used to analyze the data and profile information. For example, the disclosure utilizes the Variables Importance on Partial Least Squares (PLS) projections (VIP) is a variable selection method based on the Canonical Powered PLS (CPPLS) regression. The CPPLS algorithm assumes that the column space of X has a subspace of dimension M containing all information relevant for predicting y (known as the relevant subspace). The different strategies for PLS-based variable selection are usually based on a rotation of the standard solution by a manipulation of the PLS weight vector (w) or the regression coefficient vector, b.

The VIP method selects variables by calculating the VIP score for each variable and excluding all the variables with VIP score below a predefined threshold u (typically u=1). All the parameters that provide an increase in the predictive ability of the model are retained.

The VIP score for the variable j is defined as:

$${VIP}_{j} = \sqrt{\frac{p}{{\sum }_{m = 1}^{M} SS ( {b}_{m} \cdot {t}_{m} )} \cdot {{\sum }_{m = 1}}^{M} {w}_{mj}^{2} \cdot SS ( {b}_{m} \cdot {t}_{m} )}$$

where p is the number of variables, M the number of retained latent variables, wₘⱼ the PLS weight of the j-th variable for the m-th latent variable and SS(bₘ·tₘ) is the percentage of y explained by the m-th latent variable.

The VIP value is namely a weighted sum of squares of the PLS weights (w), which takes into account the explained variance of each PLS dimension. The “greater than one” rule is generally used as a criterion for variable selection because the average of squared VIP scores is equal to 1. Thus, in the tables and data presented herein the VIP value is based upon the foregoing.

In some embodiments, the provided methods and assays allow for the diagnosis or determination of a risk for a particular disease or disorder (e.g., PTSD, TBI, acute stress disorders and autism spectrum disorders). The disclosure also provides kits for carrying out the methods of the disclosure. The kits can include, for example, a collection device, a collection storage vial, buffers useful for collecting and storing a sample, control small molecule metabolites in a predetermined amount and the like.

In one embodiment, the disclosure provides a PTSD small molecule metabolite profile and PTSD metabolomics profile, and methods and assays for assessing the amounts or levels of metabolites within the profile and determining the presence or absence of alterations in the pathways in the profile in a subject. The PTSD metabolomics profile and such methods and assays in some embodiments can be used to determine presence or risk of other diseases and disorders such as, but not limited to acute stress disorder. The PTSD metabolomics profile comprises a plurality of metabolic pathways and each pathway comprises one or more small molecule metabolites that make up the PTSD small molecule metabolite profile. Although a large number of pathways can be used in the determining the presence or risk of PTSD, a smaller subset is sufficient. For example, in one embodiment, aberrant amounts of at least 2 small molecule metabolites in at least 8 pathways selected from the group consisting of a phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway, is indicative of the presence or risk of PTSD. Thus, in one embodiment, a PTSD metabolomics profile includes 8 pathways selected from the group consisting of phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway. In another embodiment, a PTSD metabolomics profile includes 9-10 pathways selected from the group consisting of phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway. In another embodiment, a PTSD metabolomics profile includes 11-12 pathways selected from the group consisting of phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway. In another embodiment, a PTSD metabolomics profile includes 13-14 pathways selected from the group consisting of phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway. In yet another embodiment, a PTSD metabolomics profile includes 15-16 pathways selected from the group consisting of phospholipid metabolic pathway; a fatty acid oxidation and synthesis metabolic pathway; a purine metabolic pathway; a bioamine and neurotransmitter metabolic pathway; a microbiome metabolic pathway; a sphingolipid metabolic pathway; a cholesterol, cortisol, non-gonadal steroid metabolic pathway; a pyrimidine metabolic pathway; a 3- and 4-carbon amino acid metabolic pathway; a branch chain amino acid metabolic pathway; a tryptophan, kynurenine, serotonin, melatonin metabolic pathway; a tyrosine and phenylalanine metabolic pathway; a SAM, SAH, methionine, cysteine, glutathione metabolic pathway; an eicosanoid and resolvin metabolic pathway; a pentose phosphate, gluconate metabolic pathway; and a vitamin A, carotenoid metabolic pathway.

Additional, selectivity and specificity of the measurements can be increased by including additional pathways. For example, in another embodiment, the PTSD metabolomics profile includes 17-19 metabolic pathways including the fatty acid oxidation and synthesis pathway; the vitamin A/carotenoid pathway; the tryptophan, kynurenine, serotonin, melatonin pathway; the vitamin B3 pathway; amino acid metabolic pathway; tyrosine/phenylalanine metabolic pathway; microbiome metabolic pathway; bioamines and neurotransmitter metabolic pathway; SAM, SAH, methionine, cysteine, glutathione metabolic pathway; food source, additives, preservatives, coloring and dyes; purine metabolic pathway; sphingolipid metabolic pathway; bile salt metabolic pathway; pyrimidine metabolic pathway; cholesterol, cortisol, non-gonadal steroid metabolic pathway; 1-carbon, folate, formate, clycine, serine metabolic pathway; vitamin B5 metabolic pathway; eicosanoid and resolving metabolic pathway; and phospholipid metabolic pathway.

In some embodiments, the metabolic activity and/or presence or alteration of individual pathways in a PTSD metabolomics profile are measured by assessing the amount of one or more small molecule metabolites in the respective individual pathways. Table 2A-B list exemplary pathways and exemplary small molecule metabolite, the detection of which can indicate pathway activities and/or alteration state.

TABLE 2A
List of pathways and metabolites measured
per pathway in some examples.

| | Measured |
|---|---|
| | Metabolites in |
| Pathway Name | the Pathway (N) |
| Phospholipid Metabolism | 109 |
| Fatty Acid Oxidation and Synthesis | 38 |
| Purine Metabolism | 35 |
| Bioamines and Neurotransmitter Metabolism | 13 |
| Microbiome Metabolism | 26 |
| Sphingolipid Metabolism | 74 |
| Cholesterol, Cortisol, Non-Gonadal Steroid | 20 |
| Metabolism | |
| Pyrimidine Metabolism | 26 |
| Amino Acid Metabolism (not otherwise covered) | 4 |
| Branch Chain Amino Acid Metabolism | 11 |
| Tryptophan, Kynurenine, Serotonin, Melatonin | 9 |
| Metabolism | |
| Tyrosine and Phenylalanine Metabolism | 4 |
| SAM, SAH, Methionine, Cysteine, Glutathione | 20 |
| Metabolism | |
| Eicosanoid and Resolvin Metabolism | 22 |
| Pentose Phosphate, Gluconate Metabolism | 9 |
| Vitamin A (Retinol), Carotenoid Metabolism | 3 |
| GABA, Glutamate, Arginine, Ornithine, Proline | 6 |
| Metabolism | |
| Vitamin B3 (Niacin, NAD+) Metabolism | 6 |
| Food Sources, Additives, Preservatives, Colorings, and | 2 |
| Dyes | |
| Bile Salt Metabolism | 7 |
| 1-Carbon, Folate, Formate, Glycine, Serine | 5 |
| Metabolism | |
| Vitamin B5 (Pantothenate, CoA) Metabolism | 1 |
| Vitamin C (Ascorbate) Metabolism | 3 |
| Amino-Sugar, Galactose, & Non-Glucose Metabolism | 5 |
| Vitamin B12 (Cobalamin) Metabolism | 4 |
| Histidine, Histamine, Carnosine Metabolism | 5 |
| Vitamin D (Calciferol) Metabolism | 2 |
| Isoleucine, Valine, Threonine, or Methionine | 2 |
| Metabolism | |
| Taurine, Hypotaurine Metabolism | 1 |
| Lysine Metabolism | 3 |

TABLE 2B
PTSD small molecule metabolite profile (30 metabolites); mass fragment criteria

| | | | VIP | PTSD/ | Source |
|---|---|---|---|---|---|
| No. | Chemical Name | Pathway Name | Score | Control | Temp (° C.) |
| 1 | 2-Octenoylcarnitine | Fatty Acid Oxidation and Synthesis | 4.056 | 1.448325507 | 500 |
| 2 | Retinol | Vitamin A (Retinol), Carotenoid Metabolism | 2.4319 | 0.8755181 | 500 |
| 3 | L-Tryptophan | Tryptophan, Kynurenine, Serotonin, Melatonin Metabolism | 2.3793 | 0.926512298 | 500 |
| 4 | Nicotinamide N-oxide | Vitamin B3 (Niacin, NAD+) Metabolism | 2.318 | 1.394824009 | 500 |
| 5 | Alanine | Amino Acid Metabolism (not otherwise covered) | 2.2815 | 0.905483428 | 500 |
| 6 | L-Tyrosine | Tyrosine and Phenylalanine Metabolism | 2.2207 | 0.904852776 | 500 |
| 7 | 3-Hydroxyanthranilic acid | Microbiome Metabolism | 2.1939 | 1.253125607 | 500 |
| 8 | N-Acetyl-L-aspartic acid | Bioamines and Neurotransmitter Metabolism | 2.1731 | 1.314277177 | 500 |
| 9 | Sarcosine | SAM, SAH, Methionine, Cysteine, Glutathione Metabolism | 2.1722 | 0.912096618 | 500 |
| 10 | N-Acetylaspartylglutamic acid | Bioamines and Neurotransmitter Metabolism | 2.0357 | 1.166149151 | 500 |
| 11 | Methylcysteine | Food Sources, Additives, Preservatives, Colorings, | 2.013 | 0.910113809 | 500 |
| | | and Dyes | | | |
| 12 | AICAR | Purine Metabolism | 2.012 | 1.150232677 | 500 |
| 13 | SM(d18:1/12:0) | Sphingolipid Metabolism | 1.9931 | 0.891748792 | 500 |
| 14 | Oleic acid | Fatty Acid Oxidation and Synthesis | 1.9581 | 1.296895361 | 500 |
| 15 | Docosahexaenoic acid | Fatty Acid Oxidation and Synthesis | 1.9237 | 1.212057593 | 500 |
| 16 | Glycocholic acid | Bile Salt Metabolism | 1.8912 | 0.717261659 | 500 |
| 17 | Guanosine monophosphate | Purine Metabolism | 1.8835 | 0.907181168 | 500 |
| 18 | Cytidine | Pyrimidine Metabolism | 1.8298 | 0.745451641 | 500 |
| 19 | SM(d18:1/22:0 OH) | Sphingolipid Metabolism | 1.8293 | 0.896532243 | 500 |
| 20 | Xanthine | Purine Metabolism | 1.8238 | 0.853260412 | 500 |
| 21 | Indoleacrylic acid | Microbiome Metabolism | 1.8146 | 0.938473106 | 500 |
| 22 | 7-ketocholesterol | Cholesterol, Cortisol, Non-Gonadal Steroid Metabolism | 1.8067 | 0.79813196 | 500 |
| 23 | 3-Hydroxyhexadecanoylcarnitine | Fatty Acid Oxidation and Synthesis | 1.7857 | 0.883375243 | 500 |
| 24 | Linoleic acid | Fatty Acid Oxidation and Synthesis | 1.7491 | 1.240076341 | 500 |
| 25 | Adenosine monophosphate | Purine Metabolism | 1.7135 | 0.800863804 | 500 |
| 26 | L-Serine | 1-Carbon, Folate, Formate, Glycine, Serine Metabolism | 1.7119 | 1.114712881 | 500 |
| 27 | Pantothenic acid | Vitamin B5 (Pantothenate, CoA) Metabolism | 1.7056 | 1.196600269 | 500 |
| 28 | Arachidonic Acid | Eicosanoid and Resolvin Metabolism | 1.6973 | 1.171032978 | 500 |
| 29 | PC(26:1) | Phospholipid Metabolism | 1.6424 | 1.413289444 | 500 |
| 30 | Uracil | Pyrimidine Metabolism | 1.6358 | 0.88204525 | 500 |

| | Electrospray | | | Retention | | | | |
|---|---|---|---|---|---|---|---|---|
| No. | Voltage | Q1 Mass | Q3 Mass | Time (min) | DP | EP | CE | CXP |
| 1 | 5500 | 286.3 | 85 | 8.8 | 49.64 | 10.22 | 35.83 | 16.99 |
| 2 | 5500 | 269.2 | 91 | 2.83 | 85 | 10 | 63.17 | 14.62 |
| 3 | 5500 | 205 | 146 | 12.66 | 70 | 10 | 21 | 12 |
| 4 | 5500 | 139.1 | 106 | 7.52 | 85 | 10 | 28 | 4 |
| 5 | 5500 | 90.1 | 44.2 | 13.91 | 93 | 10 | 13 | 10 |
| 6 | 5500 | 182 | 136.07 | 14.17 | 93 | 10 | 37 | 10 |
| 7 | 5500 | 154.1 | 80 | 4.82 | 35 | 10 | 39 | 10 |
| 8 | −4500 | 173.7 | 88 | 21.3 | −56.2 | −9.94 | −22.6 | −9 |
| 9 | 5500 | 90.09 | 44 | 14.39 | 93 | 10 | 19.11 | 17.26 |
| 10 | −4500 | 303 | 128.1 | 21.59 | −58.17 | −7.89 | −26.54 | −7.83 |
| 11 | 5500 | 136.02 | 119.02 | 13.67 | 93 | 10 | 12 | 10 |
| 12 | −4500 | 257 | 125 | 10.37 | −61 | −8.85 | −22.3 | −10.07 |
| 13 | −4500 | 647.67 | 184.1 | 8.8 | 121 | 10.33 | 56.8 | 25.04 |
| 14 | −4500 | 281.2 | 71 | 10.28 | −128 | −10 | −68 | −28 |
| 15 | −4500 | 327.4 | 283.4 | 10.16 | −115.8 | −8.01 | −19.8 | −15.51 |
| 16 | −4500 | 464.3 | 74 | 14.24 | −95 | −10 | −60 | −10 |
| 17 | 5500 | 364 | 152 | 21.53 | 93 | 10 | 19 | 10 |
| 18 | 5500 | 244 | 112 | 9.61 | 93 | 10 | 12 | 10 |
| 19 | −4500 | 787.8 | 79 | 8.13 | −120 | −10 | −100 | −10 |
| 20 | −4500 | 151.11 | 108 | 15.93 | −93 | −10 | −23 | −10 |
| 21 | −4500 | 186 | 142.03 | 12.66 | −93 | −10 | −20 | −10 |
| 22 | 5500 | 401.6 | 383 | 2.82 | 180 | 10 | 35 | 13 |
| 23 | 5500 | 416.6 | 85 | 2.93 | 49.64 | 10.22 | 35.83 | 16.99 |
| 24 | −4500 | 279.4 | 59 | 10.33 | −120 | −10 | −40 | −18.92 |
| 25 | 5500 | 348.22 | 136 | 21.6 | 50.34 | 10 | 27.16 | 20.8 |
| 26 | 5500 | 106 | 60 | 14.65 | 93 | 10 | 13 | 10 |
| 27 | −4500 | 218 | 146 | 14.9 | −90 | −10 | −19 | −10 |
| 28 | −4500 | 303.4 | 259.5 | 10.25 | −110 | −10 | −21.5 | −12.5 |
| 29 | 5500 | 648.5 | 184.1 | 8.52 | 80 | 10 | 20 | 11 |
| 30 | −4500 | 111.05 | 42.1 | 6.7 | −93 | −10 | −22 | −10 |

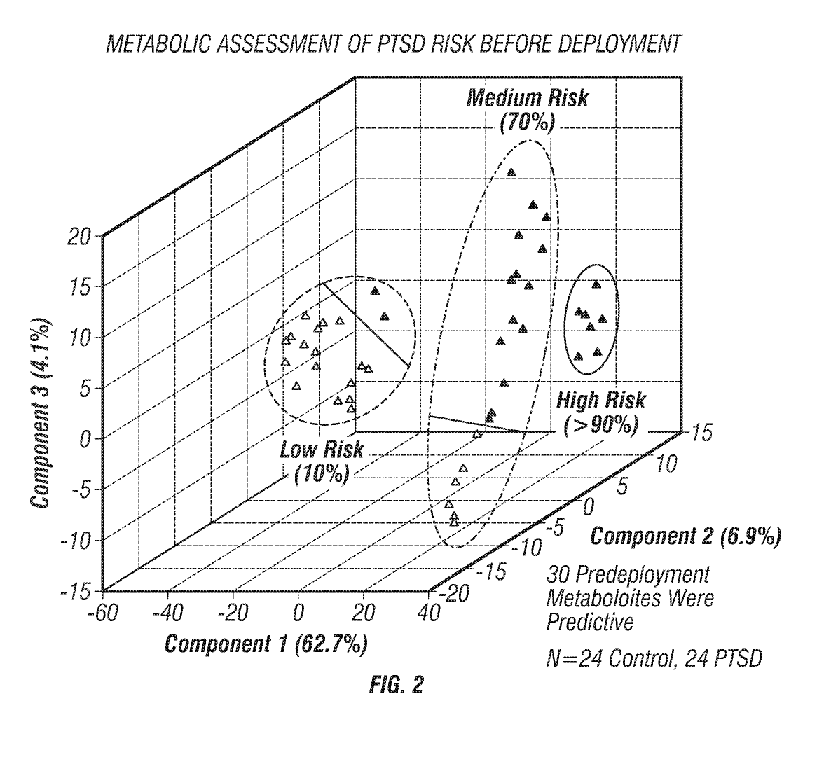

As demonstrated herein, embodiments of the provided methods were used to characterize PTSD subjects based upon metabolomics profiles. In some embodiments, the method comprises obtaining a sample from a subject (e.g., blood, urine, tissue); preparing the sample (e.g., extracting, enriching, and the like) metabolites, which can include the addition of internal standards; performing a technique to quantitate metabolites in the sample (e.g., HPLC, Mass spectroscopy, LC-MS/MS, and the like); identifying aberrant quantities of metabolites; and generating heat maps, biochemical pathway visualization or other data output for analysis. The resulting data output in some aspects is then compared to a “normal” or “control” data. Using a PTSD metabolomics profile, 20 metabolites were determined in one study to be useful in characterizing PTSD subject (see, e.g., FIG. 1). In addition, using similar methodology, 34 metabolites were useful in characterizing “at risk” subject for PTSD (see, e.g., FIG. 2).

In some embodiments, the disclosure provides an autism spectrum disorder (ASD) small molecule metabolite profile and ASD metabolomics profiles, and methods and assays for assessing the amounts or levels of metabolites within the profile and determining the presence or absence of alterations in the pathways in the profile in a subject. In some embodiment, the ASD metabolomics profile comprises a plurality of metabolic pathways and each pathway comprises one or more small molecule metabolites that make up the ASD small molecule metabolite profile. Although a large number of pathways can be used in the determining the presence or risk of ASD, a smaller subset is sufficient. For example, in one embodiment, an ASD metabolomics pathway comprises 14 metabolic pathways including purine metabolism, fatty acid oxidation, microbiome, phospholipid, eicosanoid, cholesterol/sterol, sphingolipid/gangliosides, mitochondrial, nitric oxide and reactive oxygen metabolism, branched chain amino acids, propionate and propiogenic amino acid metabolism (IVTM; Ile, Val, Thr, Met), pyrimidines, SAM/SAH/glutathione, and B6/pyridoxine metabolism. Additional, selectivity and specificity of the measurements can be increased by including additional pathways. In some embodiments, the ASD metabolomics pathway includes 14 metabolites and also includes one or more additional pathways selected from the group consisting of Vitamin B3 metabolism pathways, Cardiolipin metabolic pathways, bile salt metabolic pathways and glycolytic metabolic pathways.

The metabolic activity of each of the pathway in the ASD metabolomics profile can be measured with one or more small molecule metabolites. Tables 5 and 6, provide the pathway and the small molecule metabolite used to determine the pathway's activity.

In some embodiments, the disclosure provides methods of using metabolomics profile information to study the effectiveness of a therapy or intervention for a disease or disorder. For example, by obtaining and comparing the metabolomics profiles, amounts of metabolites, and/or alterations in pathways, from a subject having a disease or disorder and a control population, certain aberrant small molecule metabolites can be identified and their corresponding metabolic pathways identified. A therapy can then be administered or provided to a subject having the disease or disorder and a small molecule metabolite profile and metabolomics profile obtain from the subject during or after therapy. The small molecule and metabolomics profiles from the subject are analyzed with particular attention to any previously identified aberrant measurement from the disease state. A change in the small molecule metabolite or metabolomics profile of the treated subject that is more consistent with a normal control profile would be indicative of an effective therapy. By “more consistent” means that the aberrant values or pathway are trending towards or are within a desired range considered “normal” for the population.

As described in the Examples, mouse models of Fragile X and MIA were used to study the treatment of the disease model with suramine. The Fragile X mouse model is a commonly used genetic mouse model of autism. Using this genetic model, the results show that antipurinergic therapy (APT) with suramin reverses the behavioral, metabolic, and the synaptic structural abnormalities. The results support the conclusion that antipurinergic therapy is operating by a metabolic mechanism that is common to, and underlies, both the environmental MIA, and the genetic Fragile X models of ASD. This mechanism is ultimately traceable to mitochondria and is regulated by purinergic signaling.

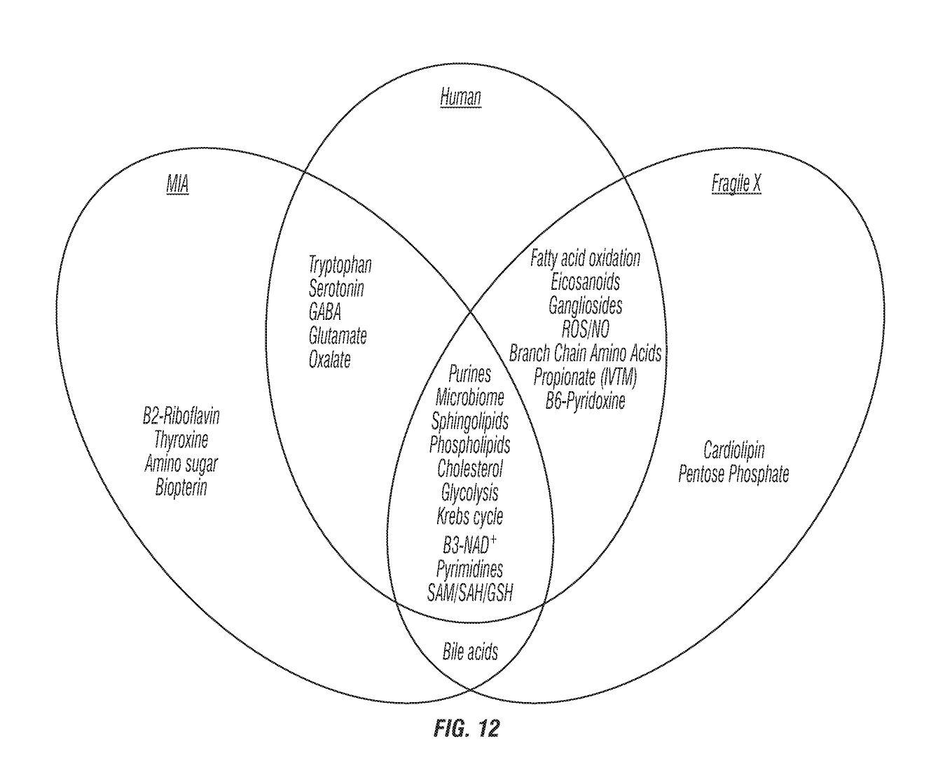

As described below, using a metabolomics profile as described herein, purine metabolism was identified as the most discriminating single metabolic pathway in the Fragile X mouse model, explaining 20% of the variance. The primary pharmacologic mechanism of action of suramin is as a competitive antagonist of extracellular ATP and other nucleotides, acting at purinergic receptors. The metabolomic data show that the major impact of suramin in the Fragile X mouse models was on purine metabolism (Table 6). In addition, a comparison of the metabolomic results for both the maternal immune activation (MIA) (Example 3) and Fragile X mouse models (Example 2) of ASD identified 11 overlapping metabolic pathways (FIG. 12). These were purines, microbiome, phospholipids, sphingolipids/gangliosides, cholesterol/sterol, bile acids, glycolysis, mitochondrial Krebs cycle, NAD+, pyrimidines, and S-adenosylmethionine/homocysteine/glutathione (SAM/SAH/GSH) metabolism. Fourteen of the 20 metabolic pathway disturbances found in the Fragile X mouse model have been described in human ASD. These include purine metabolism (Nyhan et al., 1969; Page and Coleman, 2000), fatty acid oxidation (Frye et al., 2013), microbiome (Mulle et al., 2013; Williams et al., 2011), phospholipid (Pastural et al., 2009), eicosanoid (Beaulieu, 2013; El-Ansary and Al-Ayadhi, 2012; Gorrindo et al., 2013), cholesterol/sterol (Tierney et al., 2006), sphingolipid/gangliosides (Nordin et al., 1998; Schengrund et al., 2012), mitochondrial (Graf et al., 2000; Rose et al., 2014; Smith et al., 2012), nitric oxide and reactive oxygen metabolism (Frustaci et al., 2012), branched chain amino acids (Tirouvanziam et al., 2012), propionate and propiogenic amino acid metabolism (IVTM; Ile, Val, Thr, Met) (Al-Owain et al., 2013), pyrimidines (Micheli et al., 2011), SAM/SAH/glutathione (James et al., 2008), and B6/pyridoxine metabolism (Adams et al., 2006). The upregulation of glycolysis and downregulation of mitochondrial Krebs cycle in ASD are a direct consequence of the regulated decrease in mitochondrial oxidative phosphorylation and the poised state of mitochondrial underfunction. If cellular activity is maintained, this produces the capacity for bursts of reactive oxygen species (ROS) production associated with the cell danger response. When cellular activity drops, then some cells within the mosaic that makes up a tissue may enter a hypometabolic state associated with resistance to harsh extracellular conditions and cellular persistence. In both cases fatty acid oxidation is decreased to facilitate intracellular lipid accumulation needed for persistence metabolism. The discovery that bile acid metabolism is dysregulated in both the MIA and Fragile X models has not previously been identified and opens the door for further studies on the role of bile acids in the brain under conditions of chronic stress. These data show that the metabolic disturbances in the MIA and Fragile X mouse models are similar to those found in human ASD, and provide strong support for the biochemical validity of these two mouse models.

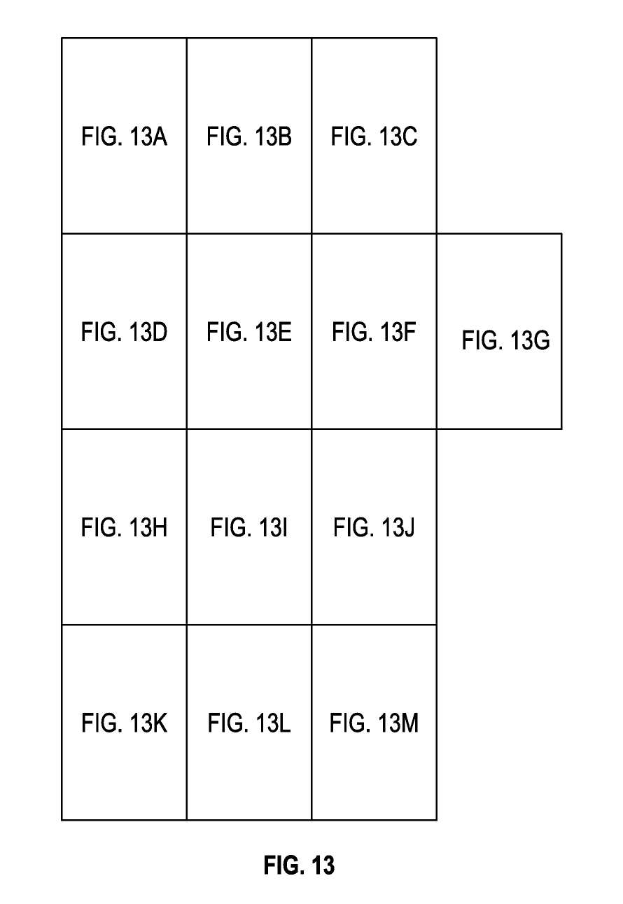

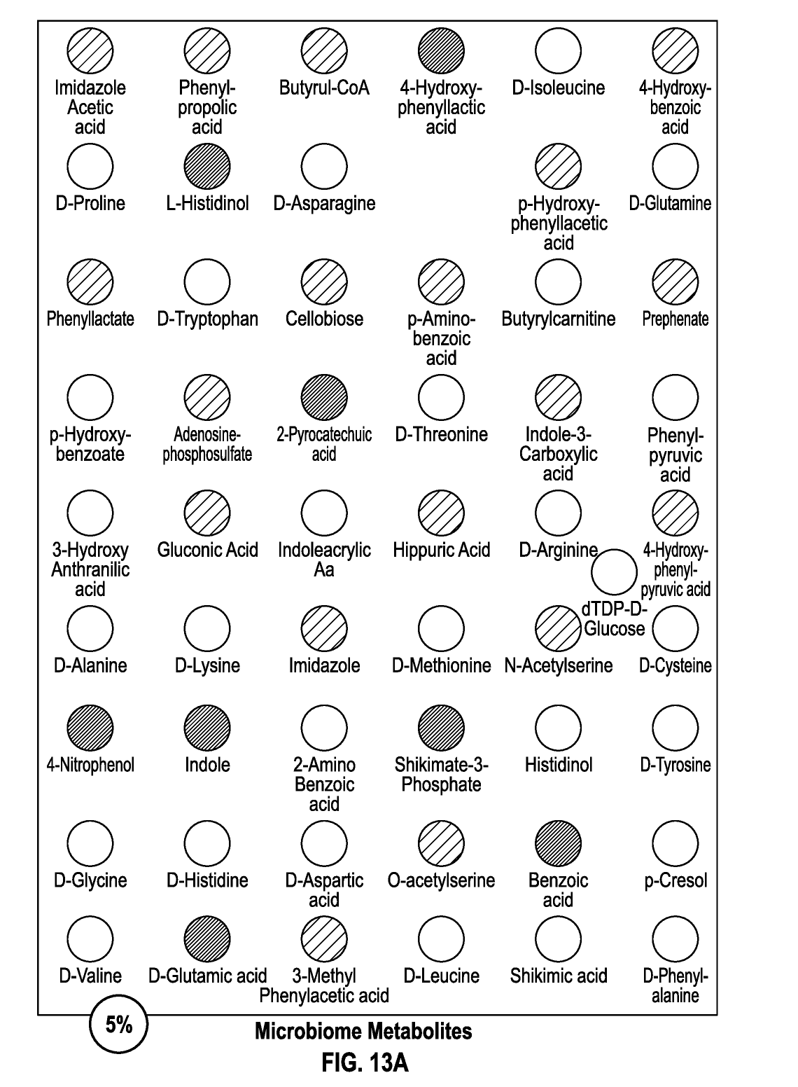

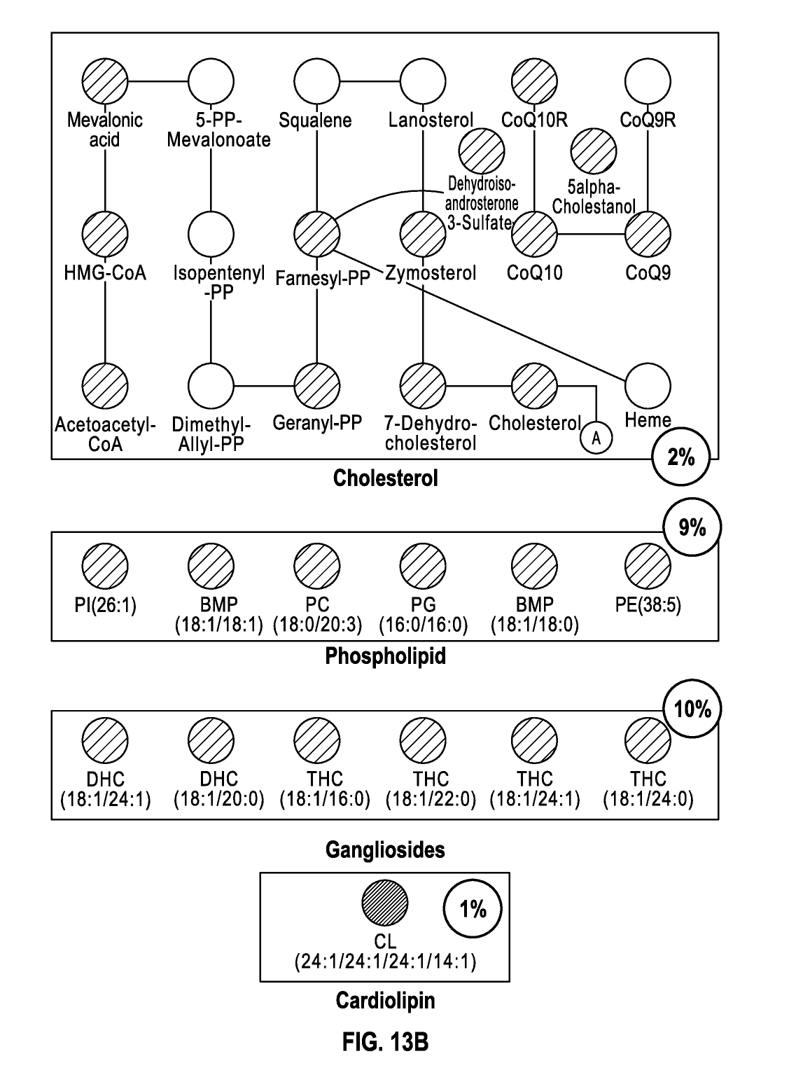

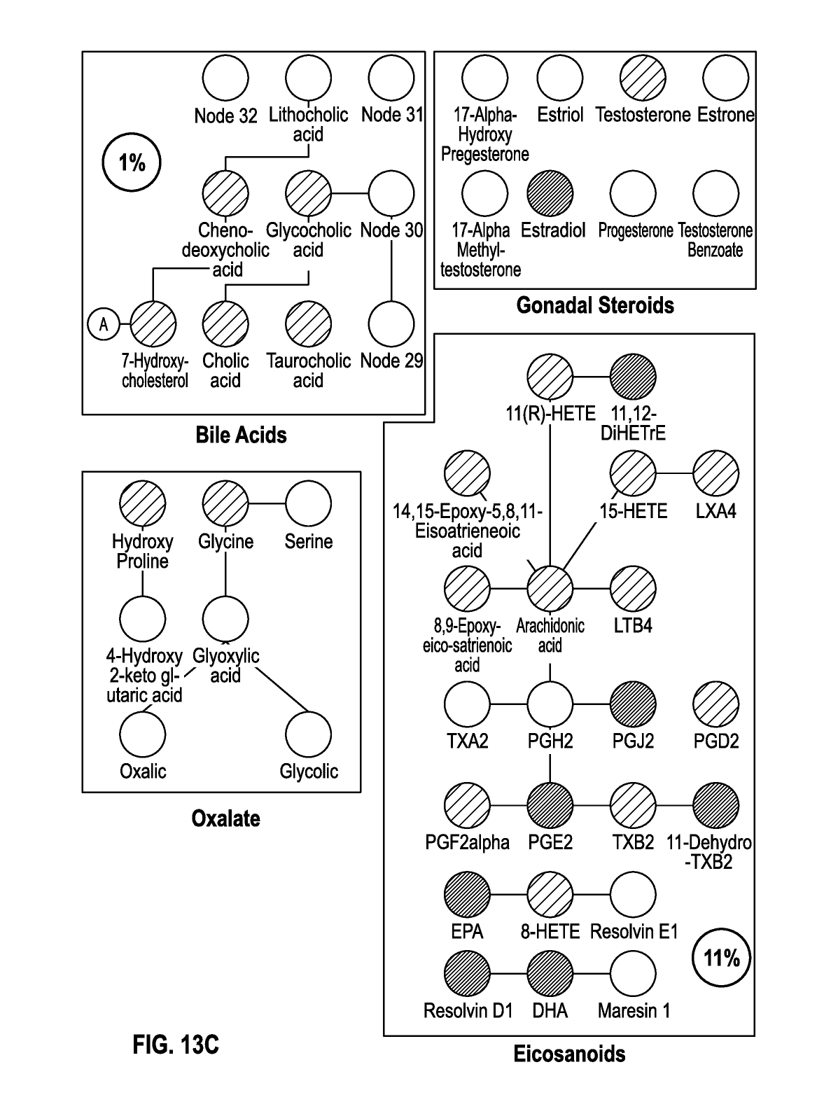

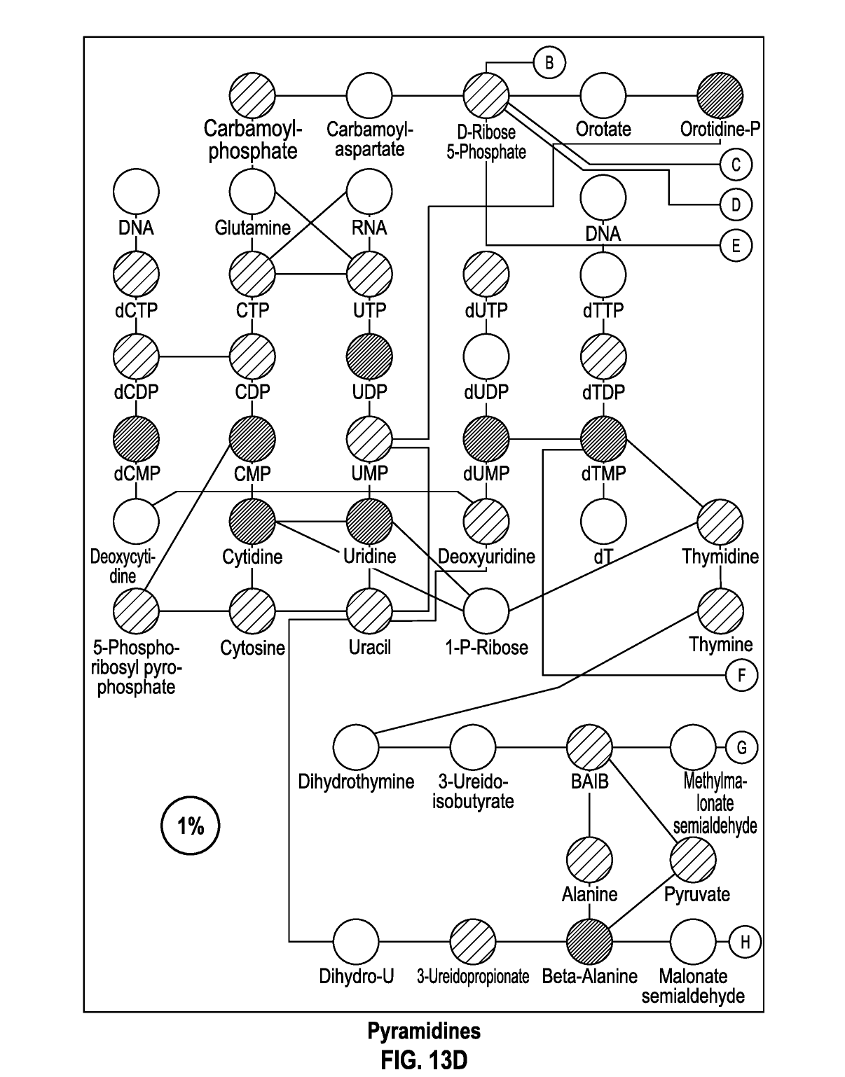

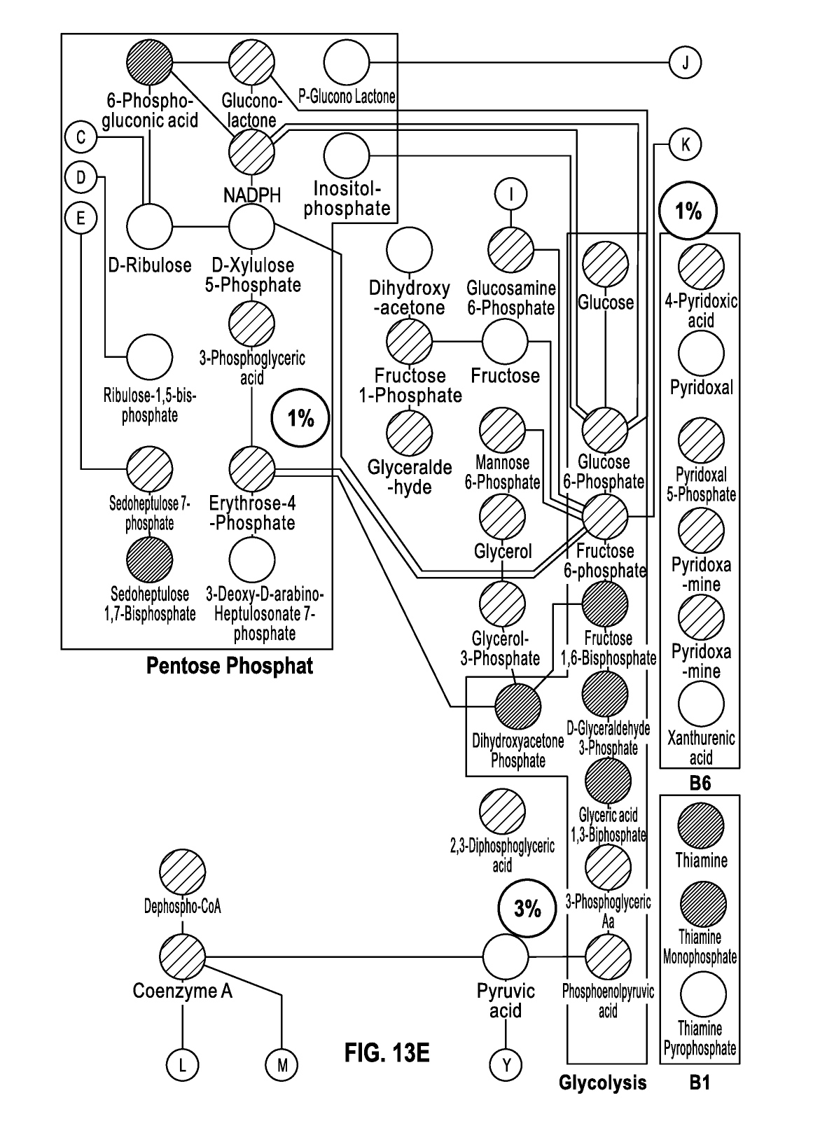

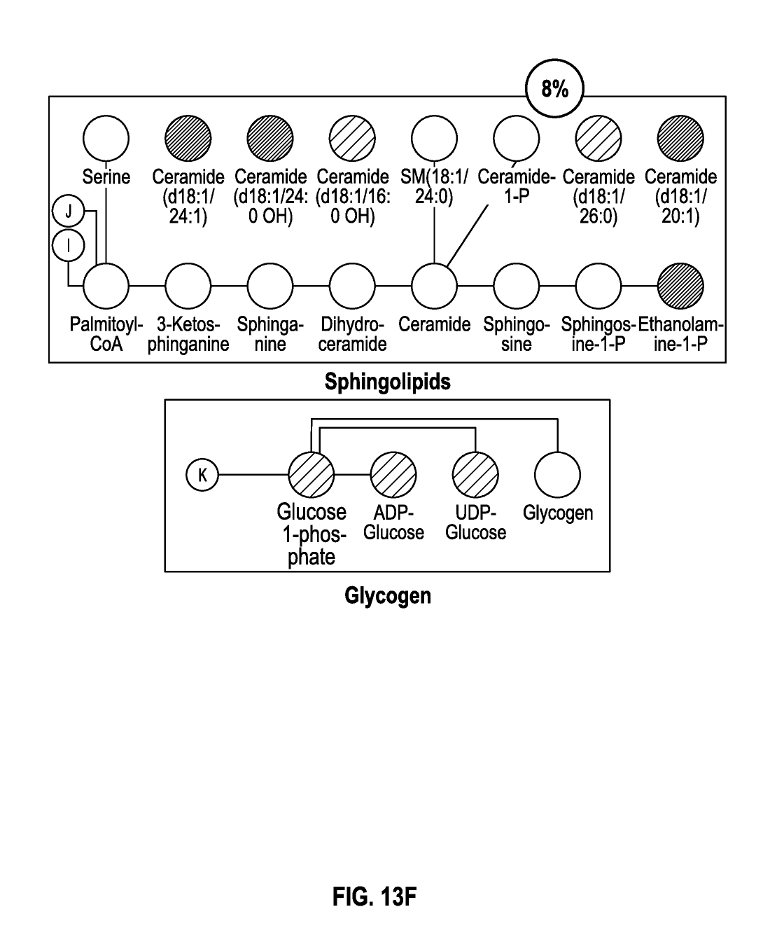

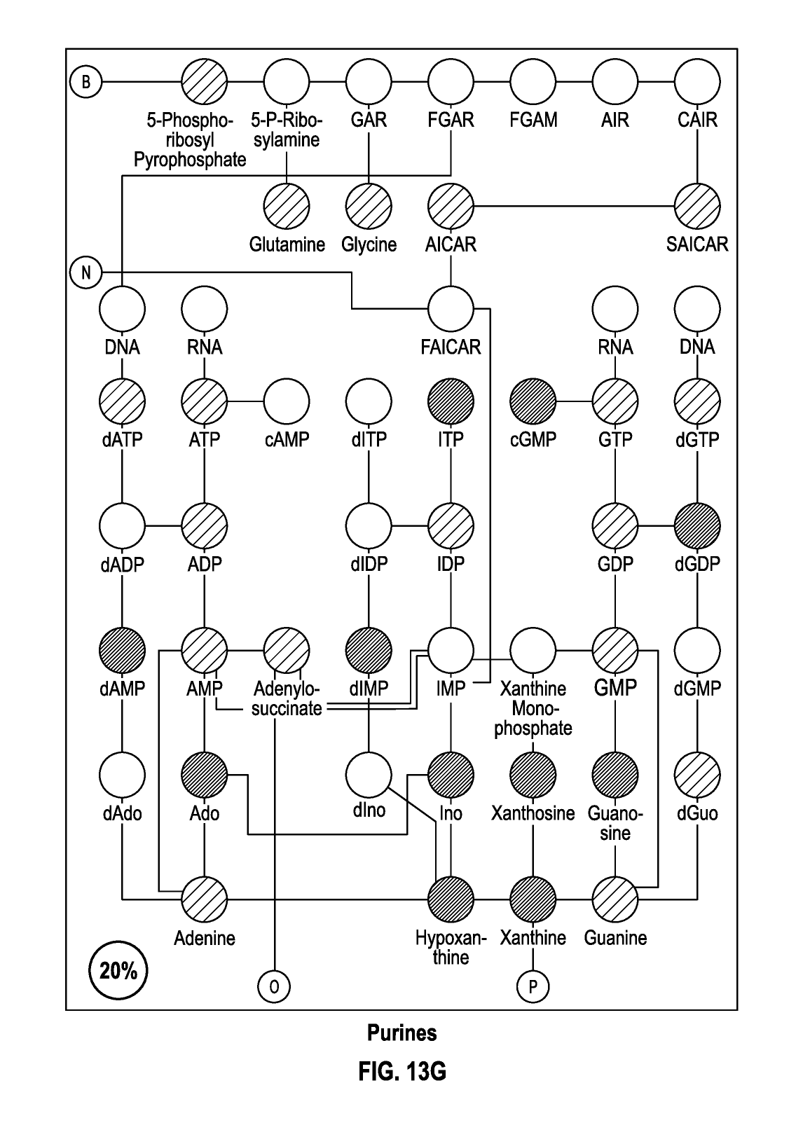

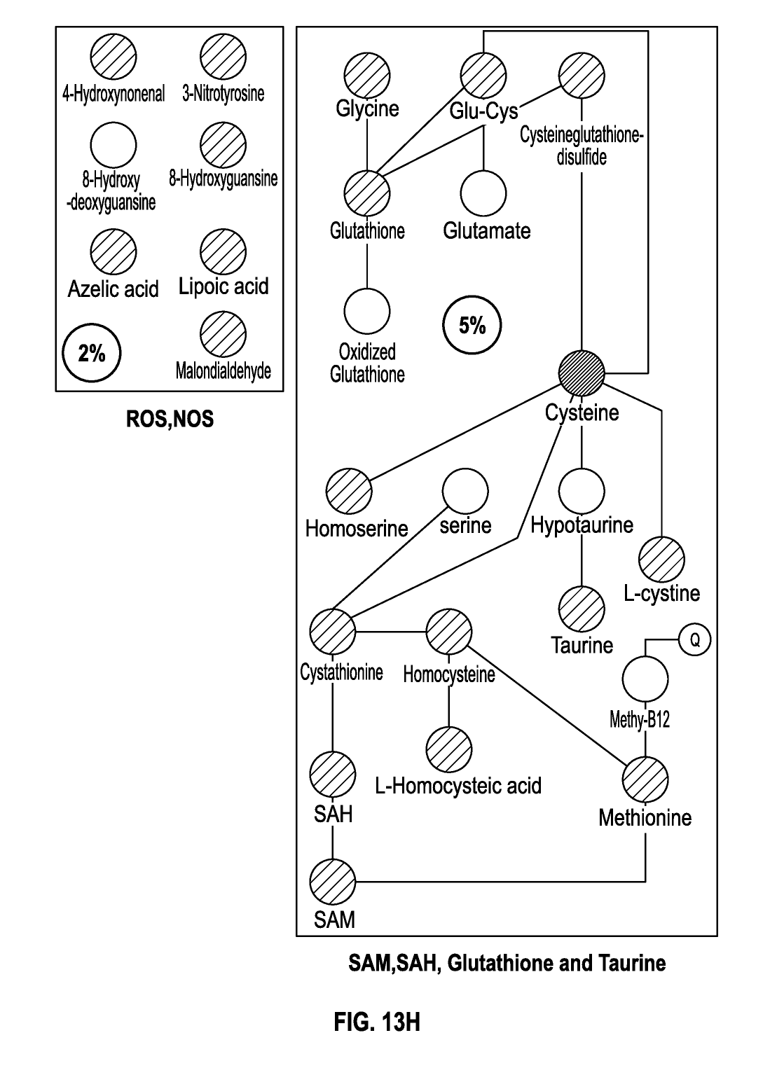

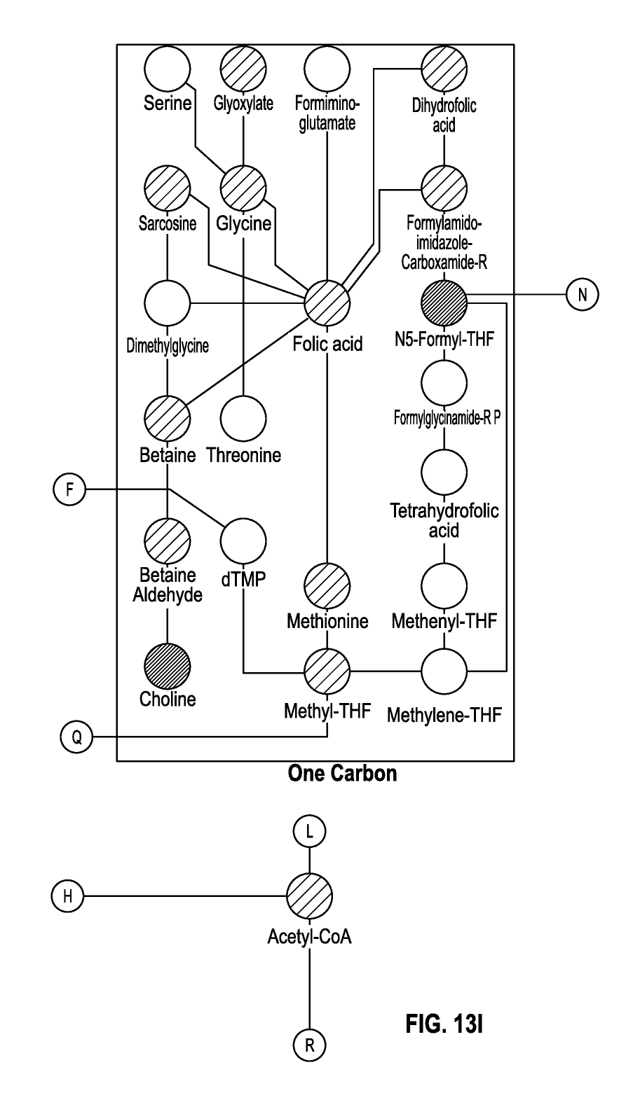

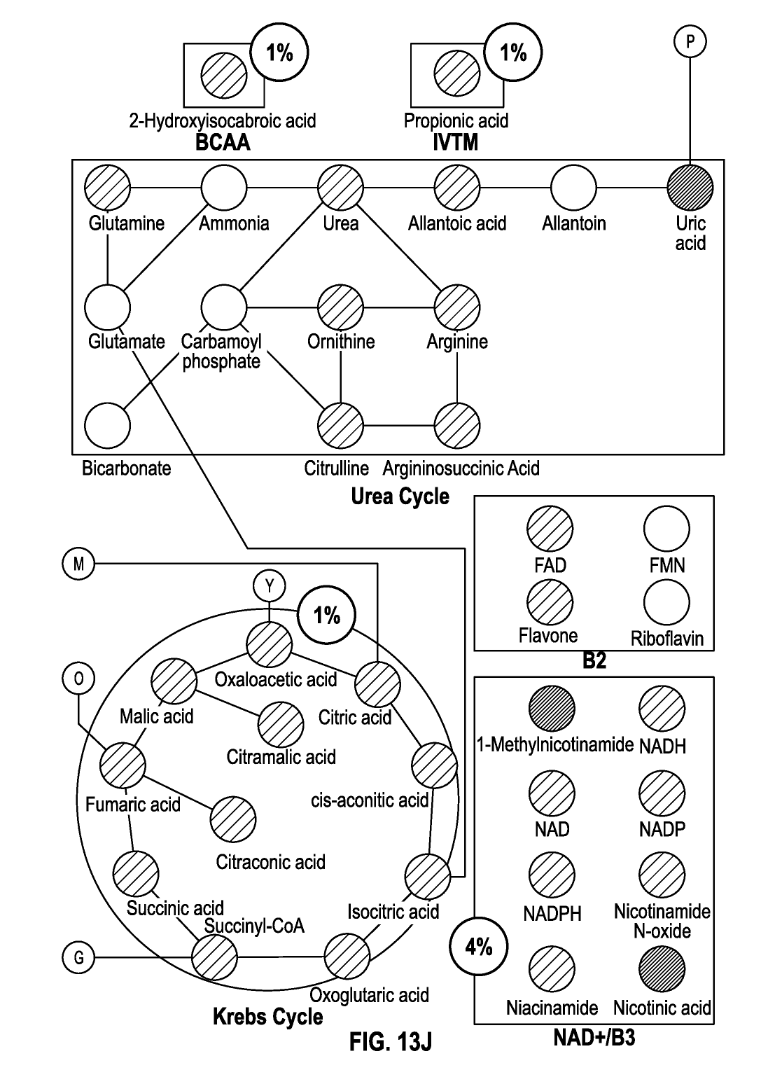

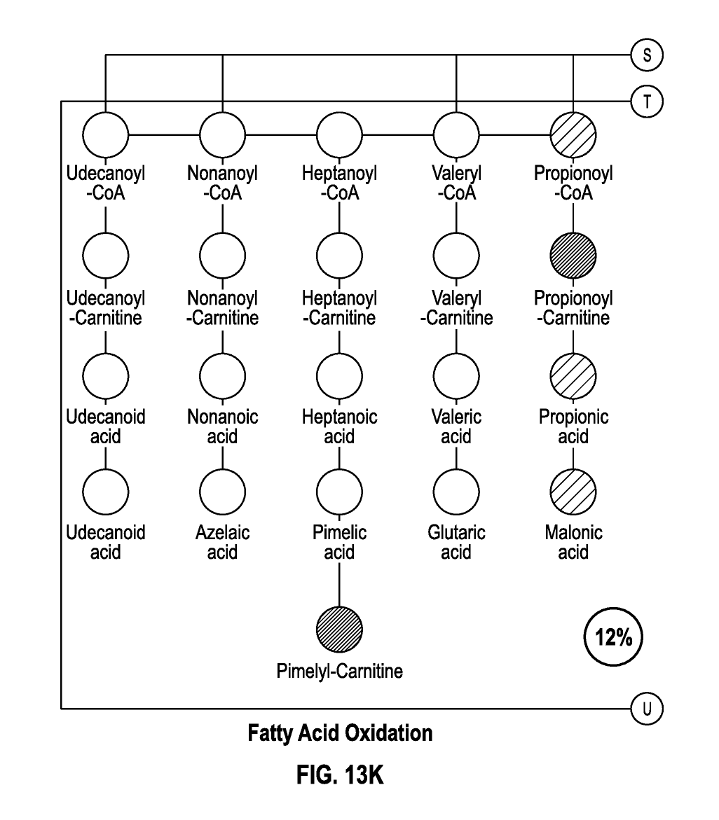

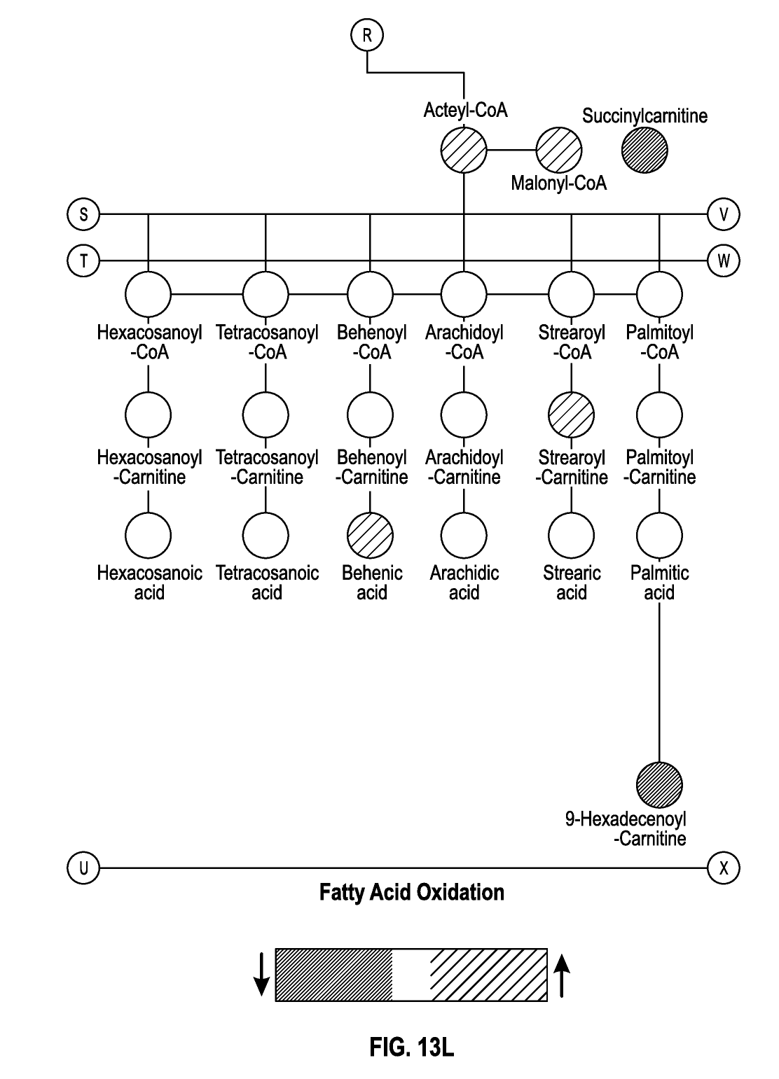

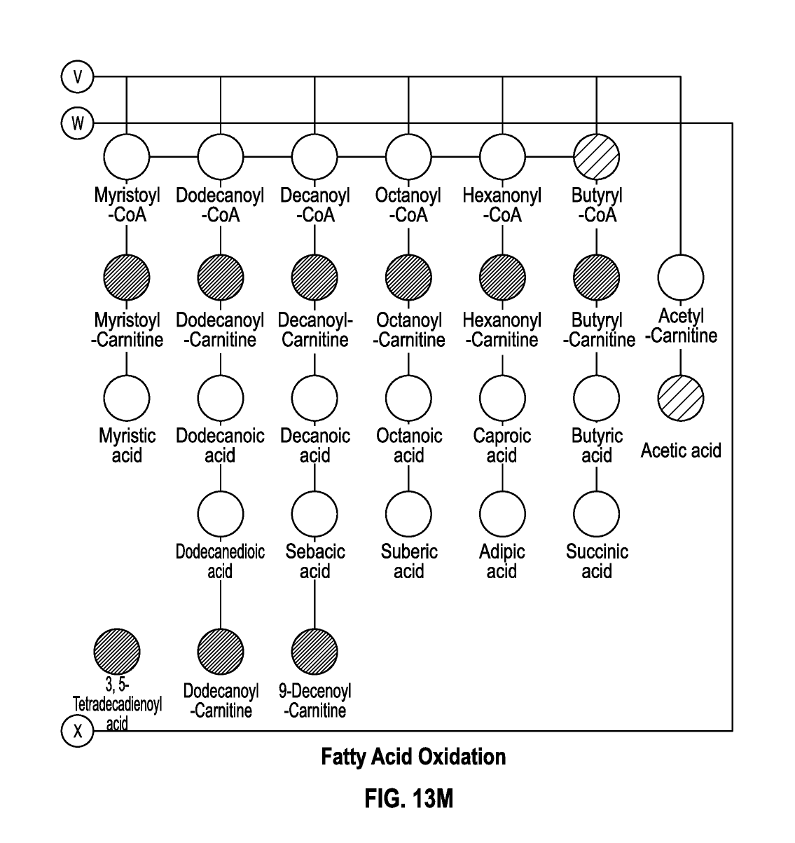

In addition, the metabolomic analysis demonstrates that disturbances in lipid metabolism are prominent in the Fragile X mouse model, and its response to treatment (Table 6, FIG. 13). Correction of purinergic signaling and purine metabolism produced concerted effects in 8 different classes of lipids that collectively explained 54% of the metabolic variance. In rank order of importance these were: fatty acid metabolism (12%), eicosanoid metabolism (11%), ganglioside metabolism (10%), phospholipid metabolism (9%), sphingolipids (8%), cholesterol/sterols (2%), cardiolipin (1%), and bile acids (1%) (Table 6). Suramin also had a significant impact on lipid metabolism in the MIA model. Four of the top 6 metabolic pathways were lipids, explaining 30% of the total metabolic variance. In rank order of importance the lipid pathways in the MIA model were: phospholipids (8%), bile acids (8%), sphingolipids (7%), and cholesterol/sterols (7%).

Several drug interventions have been successful in mitigating symptoms in the Fragile X mouse model or in human clinical trials. These include antagonists of glutamatergic (mGluR5) signaling (Michalon et al., 2014), agonists of GABAergic signaling (Henderson et al., 2012), metabolic supportive therapy with acetyl-L-carnitine (Torrioli et al., 2008), and inhibition of the metabolic control enzyme glycogen synthase kinase 3β (GSK3β) (Franklin et al., 2014). The data presented herein show that metabolic changes, in the form of altered abundance and flow of metabolites used for cell growth, repair and signaling, are driving the ship formerly thought to be controlled by neurotransmitters, protein signaling, and transcription factors. The data presented below that proteins like TDP43 and APP are decreased by antipurinergic therapy with suramin. Thus, contributing to the emerging concept of metabolic primacy in neurodevelopmental, neuropsychiatric, and neurodegenerative disease.

### EXAMPLES

### Example 1A

### PTSD Metabolomics

Broad spectrum analysis of 478 targeted metabolites from 44 biochemical pathways was performed (Table 8). In other experiments 868 metabolites form 63 pathways have been interrogated (see, e.g., Table 1). Samples were analyzed on an AB SCIEX QTRAP 5500 triple quadrupole mass spectrometer equipped with a Turbo V electrospray ionization (ESI) source, Shimadzu LC-20A UHPLC system, and a PAL CTC autosampler (AB ACIEX, Framingham, Mass., USA). Whole blood was collected into BD Microtainer tubes containing lithium heparin (Becton Dickinson, San Diego, Calif., USA, Ref#365971). Plasma was separated by centrifugation at 600 g×5 minutes at 20° C. within one hour of collection. Fresh lithium-heparin plasma was transferred to labeled tubes for storage at −80° C. for analysis. Typically 45 μl of plasma was thawed on ice and transferred to a 1.7 ml Eppendorf tube. Two and one-half (2.5) μl of a cocktail containing 35 commercial stable isotope internal standards, and 2.5 μl of 310 stable isotope internal standards that were custom-synthesized in E. coli and S. cerevisiae by metabolic labeling with ¹³C-glucose and ¹³C-bicarbonate, were added, mixed, and incubated for 10 min at room temperature to permit small molecules and vitamins in the internal standards to associate with plasma binding proteins. Macromolecules (protein, DNA, RNA, etc.) were precipitated by extraction with 4 volumes (200 μl) of cold (−20° C.), acetonitrile:methanol (50:50) (LCMS grade, Cat#LC015-2.5 and GC230-4, Burdick & Jackson, Honeywell), vortexed vigorously, and incubated on crushed ice for 10 min, then removed by centrifugation at 16,000 g×10 min at 4° C. The supernatants containing the extracted metabolites and internal standards in the resulting 40:40:20 solvent mix of acetonitrile:methanol:water were transferred to labeled cryotubes and stored at −80° C. for LC-MS/MS (liquid chromatography-tandem mass spectrometry) analysis.

LC-MS/MS analysis was performed by multiple reaction monitoring (MRM) under Analyst v1.6.1 (AB SCIEX, Framingham, Mass., USA) software control in both negative and positive mode with rapid polarity switching (50 ms). Nitrogen was used for curtain gas (set to 30), collision gas (set to high), ion source gas 1 and 2 (set to 35). The source temperature was 500° C. Spray voltage was set to −4500 V in negative mode and 5500 V in positive mode. The values for Q1 and Q3 mass-to-charge ratios (m/z), declustering potential (DP), entrance potential (EP), collision energy (CE), and collision cell exit potential (CXP) were determined and optimized for each MRM for each metabolite. Ten microliters of extract was injected by PAL CTC autosampler into a 250 mm×2 mm, 5 μm Luna NH₂ aminopropyl HPLC column (Phenomenex, Torrance, Calif., USA) held at 25° C. for chromatographic separation. The mobile phase was solvent A: 95% water with 23.18 mM NH₄OH (Sigma-Aldrich, St. Louis, Mo., USA, Fluka Cat#17837-100ML), 20 mM formic acid (Sigma, Fluka Cat#09676-100ML) and 5% acetonitrile (pH 9.44); solvent B: 100% acetonitrile. Separation was achieved using the following gradient: 0 min-95% B, 3 min-95% B, 3.1 min 80% B, 6 min 80% B, 6.1 min 70% B, 10 min 70% B, 18 min 2% B, 27 min 0% B, 32 min 0% B, 33 min 100% B, 36.1 95% B, 40 min 95% B end. The flow rate was 300 μl/min. All the samples were kept at 4° C. during analysis. The chromatographic peaks were identified using MultiQuant (v3.0, AB SCIEX), confirmed by manual inspection, and the peak areas integrated. The median of the peak area of stable isotope internal standards was calculated and used for the normalization of metabolites concentration across the samples and batches. Prior to multivariate and univariate analysis, the data were log-transformed.

The metabolites and pathways analyzed are set forth in Table 2A-B and Table 2C.

TABLE 2C
Metabolic Pathways for PTSD Diagnosis:

| | | | | | | | Fraction | | |
|---|---|---|---|---|---|---|---|---|---|
| | | | Expected | | | | of Impact | | |
| | Measured | Expected | Hits in | Observed | | Impact | (VIP) | | |
| | Metabolites | Pathway | Sample of | Hits in the | Fold | (Sum | Explained | | |
| | in the | Proportion | 85 (P * | Top 85 | Enrichment | VIP | (% of | | |
| Pathway Name | Pathway (N) | (P = N/580) | 85) | Metabolites | (Obs/Exp) | Score) | 130.7) | Up | Down |
| Phospholipid Metabolism | 109 | 0.188 | 16.0 | 12 | 0.8 | 16.1 | 12.3% | 3 | 9 |
| Fatty Acid Oxidation and Synthesis | 38 | 0.066 | 5.6 | 8 | 1.4 | 15.6 | 11.9% | 6 | 2 |
| Purine Metabolism | 35 | 0.060 | 5.1 | 10 | 1.9 | 14.7 | 11.2% | 2 | 8 |
| Bioamines and Neurotransmitter | 13 | 0.022 | 1.9 | 6 | 3.1 | 9.2 | 7.1% | 3 | 3 |
| Metabolism | | | | | | | | | |
| Microbiome Metabolism | 26 | 0.045 | 3.8 | 6 | 1.6 | 8.8 | 6.7% | 1 | 5 |
| Sphingolipid Metabolism | 74 | 0.128 | 10.8 | 5 | 0.5 | 7.6 | 5.8% | 0 | 5 |
| Cholesterol, Cortisol, Non-Gonadal Steroid | 20 | 0.034 | 2.9 | 4 | 1.4 | 5.5 | 4.2% | 1 | 3 |
| Metabolism | | | | | | | | | |
| Pyrimidine Metabolism | 26 | 0.045 | 3.8 | 3 | 0.8 | 4.5 | 3.4% | 1 | 2 |
| Amino Acid Metabolism (not otherwise | 4 | 0.007 | 0.6 | 2 | 3.4 | 3.6 | 2.8% | 0 | 2 |
| covered) | | | | | | | | | |
| Branch Chain Amino Acid Metabolism | 11 | 0.019 | 1.6 | 3 | 1.9 | 3.6 | 2.7% | 1 | 2 |
| Tryptophan, Kynurenine, Serotonin, | 9 | 0.016 | 1.3 | 2 | 1.5 | 3.5 | 2.7% | 0 | 2 |
| Melatonin Metabolism | | | | | | | | | |
| Tyrosine and Phenylalanine Metabolism | 4 | 0.007 | 0.6 | 2 | 3.4 | 3.5 | 2.7% | 0 | 2 |
| SAM, SAH, Methionine, Cysteine, | 20 | 0.034 | 2.9 | 2 | 0.7 | 3.4 | 2.6% | 0 | 2 |
| Glutathione Metabolism | | | | | | | | | |
| Eicosanoid and Resolvin Metabolism | 22 | 0.038 | 3.2 | 2 | 0.6 | 3.3 | 2.5% | 1 | 1 |
| Pentose Phosphate, Gluconate Metabolism | 9 | 0.016 | 1.3 | 2 | 1.5 | 3.2 | 2.4% | 1 | 1 |
| Vitamin A (Retinol), Carotenoid | 3 | 0.005 | 0.4 | 1 | 2.3 | 2.4 | 1.9% | 0 | 1 |
| Metabolism | | | | | | | | | |
| GABA, Glutamate, Arginine, Ornithine, | 6 | 0.010 | 0.9 | 2 | 2.3 | 2.3 | 1.8% | 1 | 1 |
| Proline Metabolism | | | | | | | | | |
| Vitamin B3 (Niacin, NAD+) Metabolism | 6 | 0.010 | 0.9 | 1 | 1.1 | 2.3 | 1.8% | 1 | 0 |
| Food Sources, Additives, Preservatives, | 2 | 0.003 | 0.3 | 1 | 3.4 | 2.0 | 1.5% | 0 | 1 |
| Colorings, and Dyes | | | | | | | | | |
| Bile Salt Metabolism | 7 | 0.012 | 1.0 | 1 | 1.0 | 1.9 | 1.4% | 0 | 1 |
| 1-Carbon, Folate, Formate, Glycine, Serine | 5 | 0.009 | 0.7 | 1 | 1.4 | 1.7 | 1.3% | 1 | 0 |
| Metabolism | | | | | | | | | |
| Vitamin B5 (Pantothenate, CoA) | 1 | 0.002 | 0.1 | 1 | 6.8 | 1.7 | 1.3% | 1 | 0 |
| Metabolism | | | | | | | | | |
| Vitamin C (Ascorbate) Metabolism | 3 | 0.005 | 0.4 | 1 | 2.3 | 1.6 | 1.2% | 1 | 0 |
| Amino-Sugar, Galactose, & Non-Glucose | 5 | 0.009 | 0.7 | 1 | 1.4 | 1.5 | 1.2% | 0 | 1 |
| Metabolism | | | | | | | | | |
| Vitamin B12 (Cobalamin) Metabolism | 4 | 0.007 | 0.6 | 1 | 1.7 | 1.2 | 1.0% | 1 | 0 |
| Histidine, Histamine, Carnosine | 5 | 0.009 | 0.7 | 1 | 1.4 | 1.2 | 0.9% | 1 | 0 |
| Metabolism | | | | | | | | | |
| Vitamin D (Calciferol) Metabolism | 2 | 0.003 | 0.3 | 1 | 3.4 | 1.2 | 0.9% | 0 | 1 |
| Isoleucine, Valine, Threonine, or | 2 | 0.003 | 0.3 | 1 | 3.4 | 1.2 | 0.9% | 1 | 0 |
| Methionine Metabolism | | | | | | | | | |
| Taurine, Hypotaurine Metabolism | 1 | 0.002 | 0.1 | 1 | 6.8 | 1.2 | 0.9% | 1 | 0 |
| Lysine Metabolism | 3 | 0.005 | 0.4 | 1 | 2.3 | 1.0 | 0.8% | 0 | 1 |
| | 475 | 82% | 70 (0.82 × | 85 | | 130.7 | 100% | 29 | 56 |
| | | (475/580) | 85) | | | | | | |

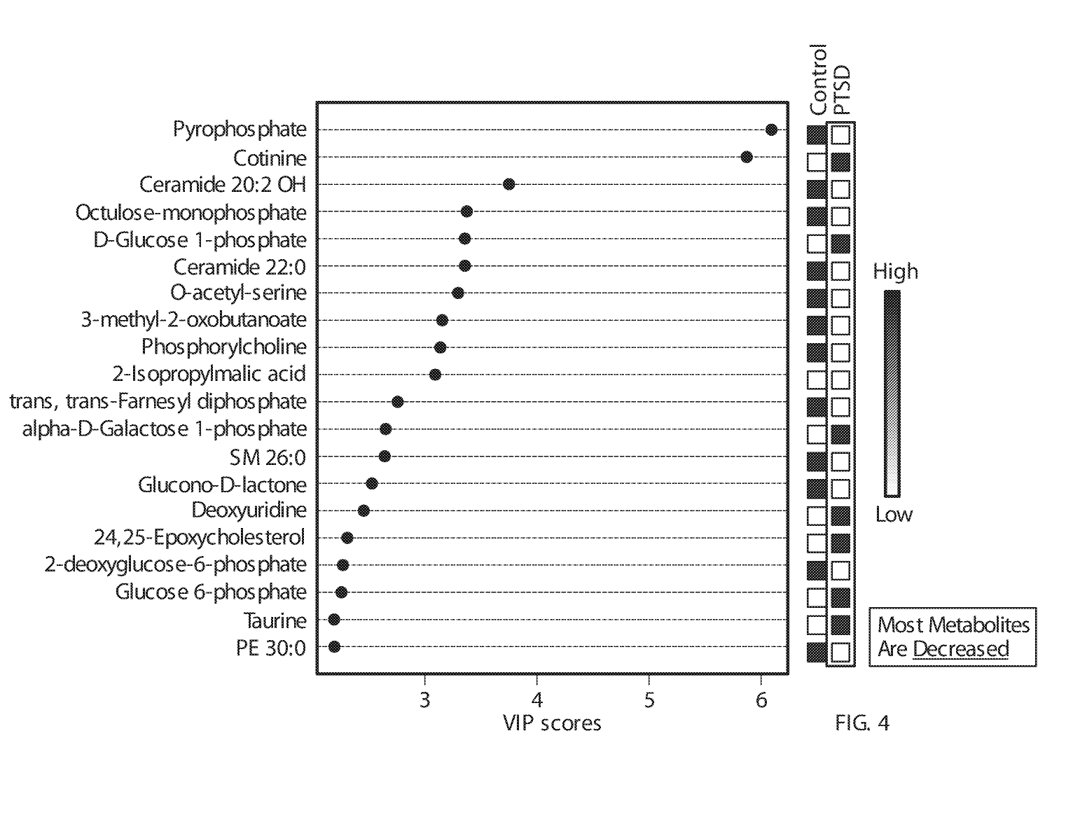

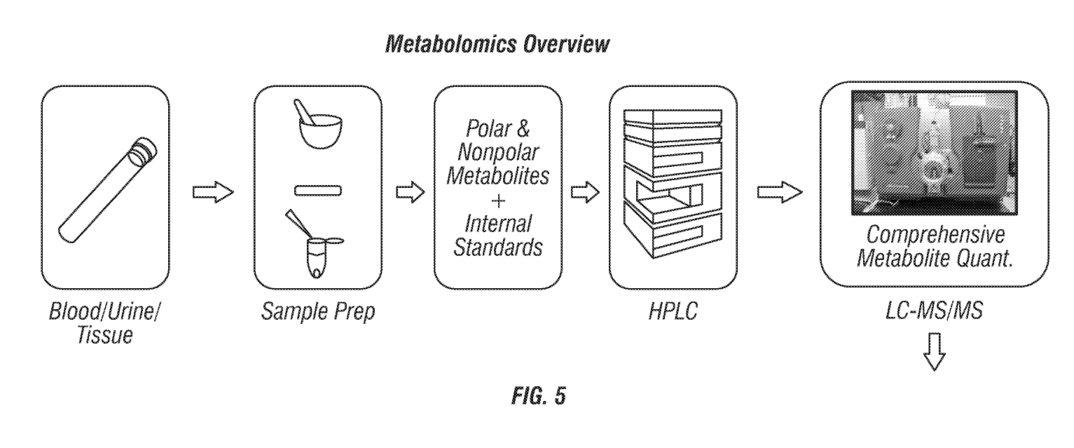

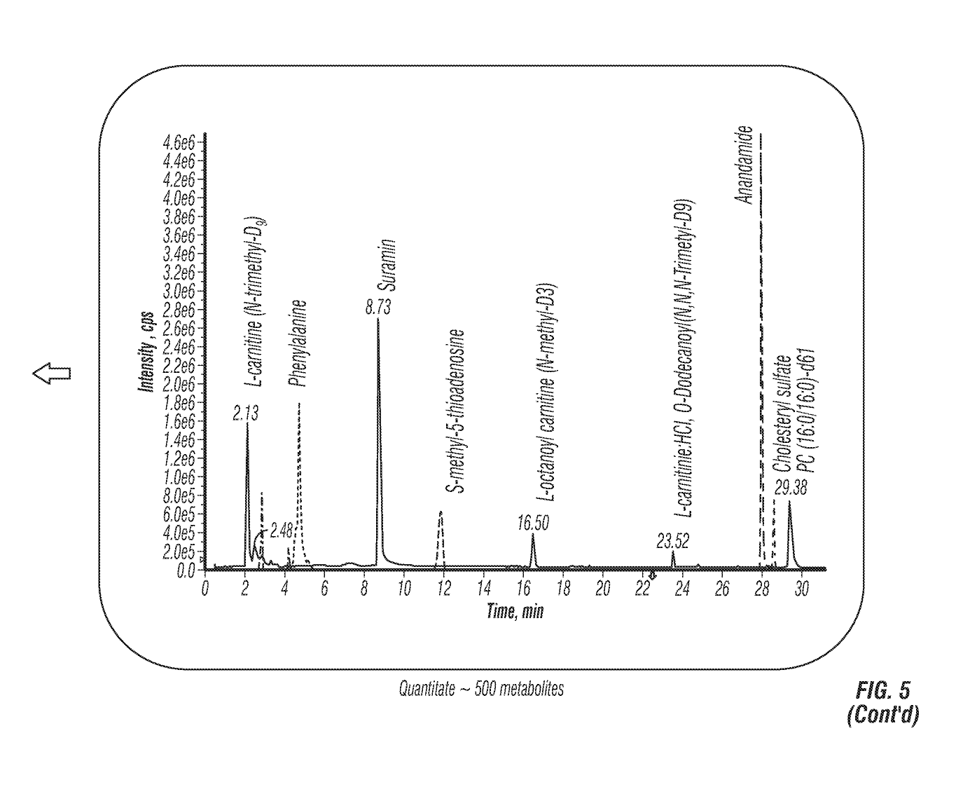

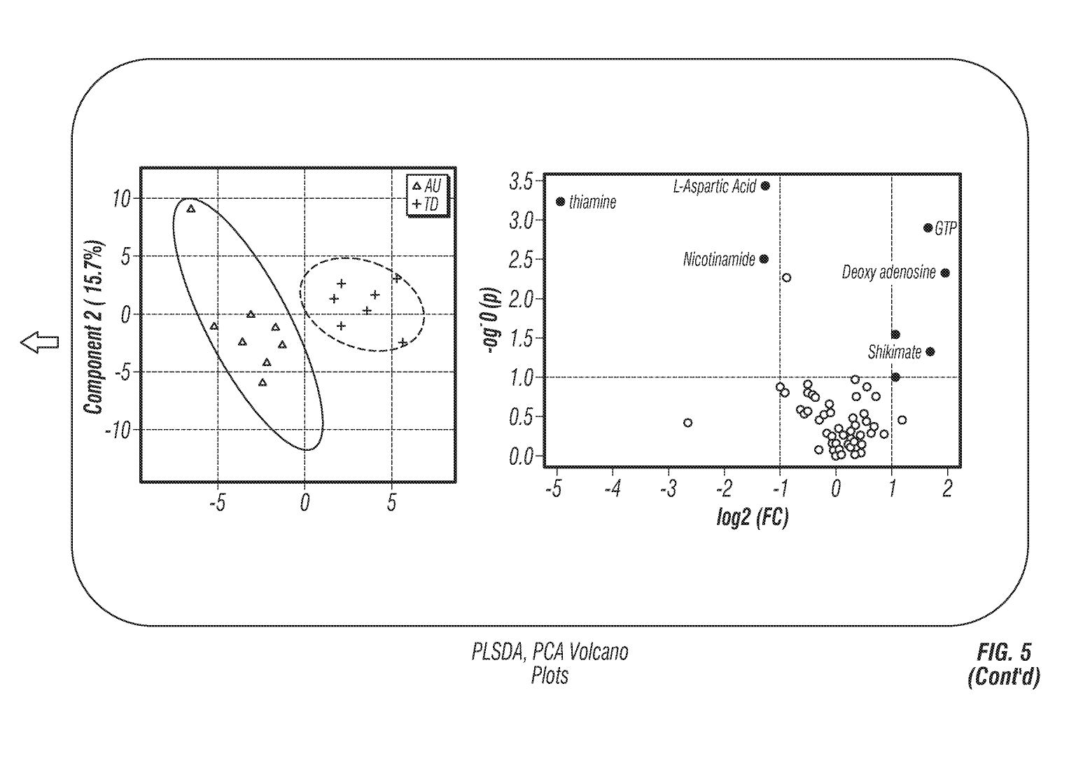

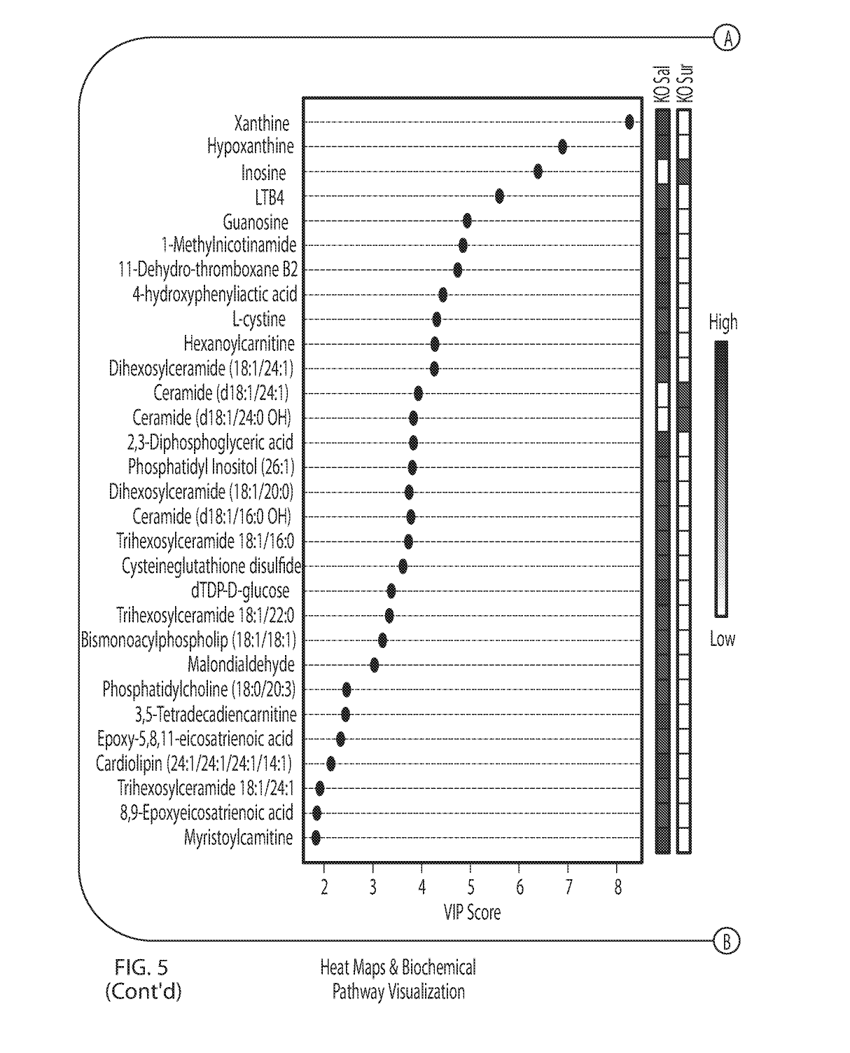

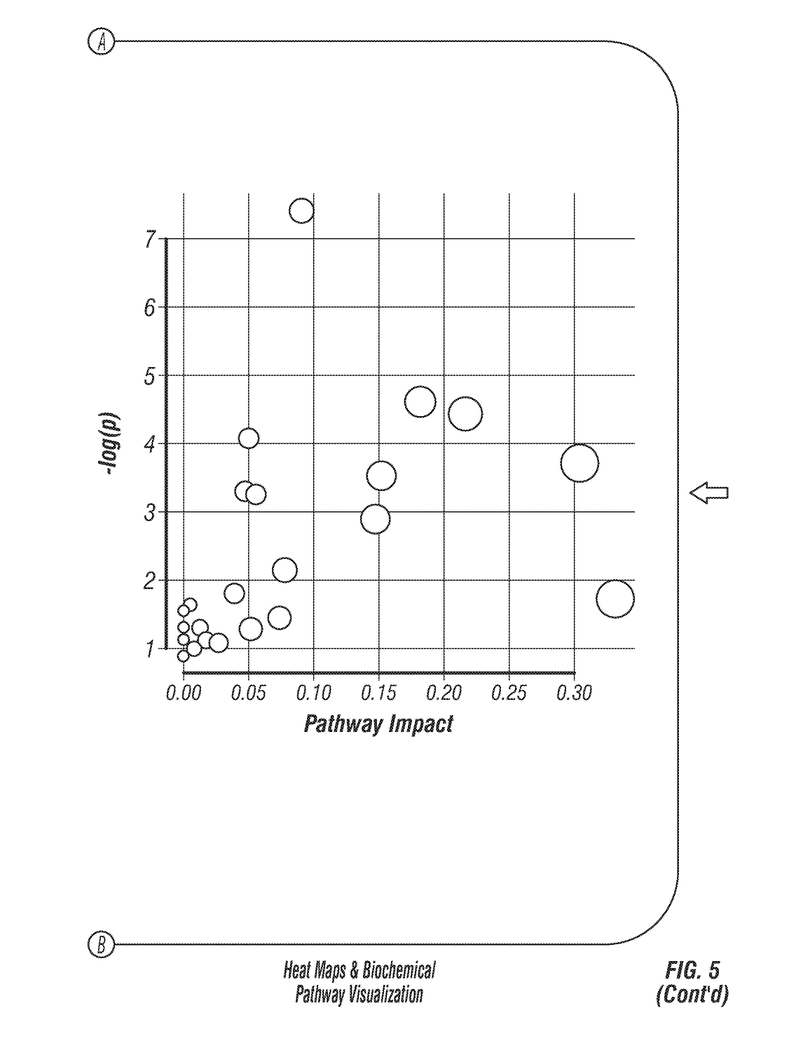

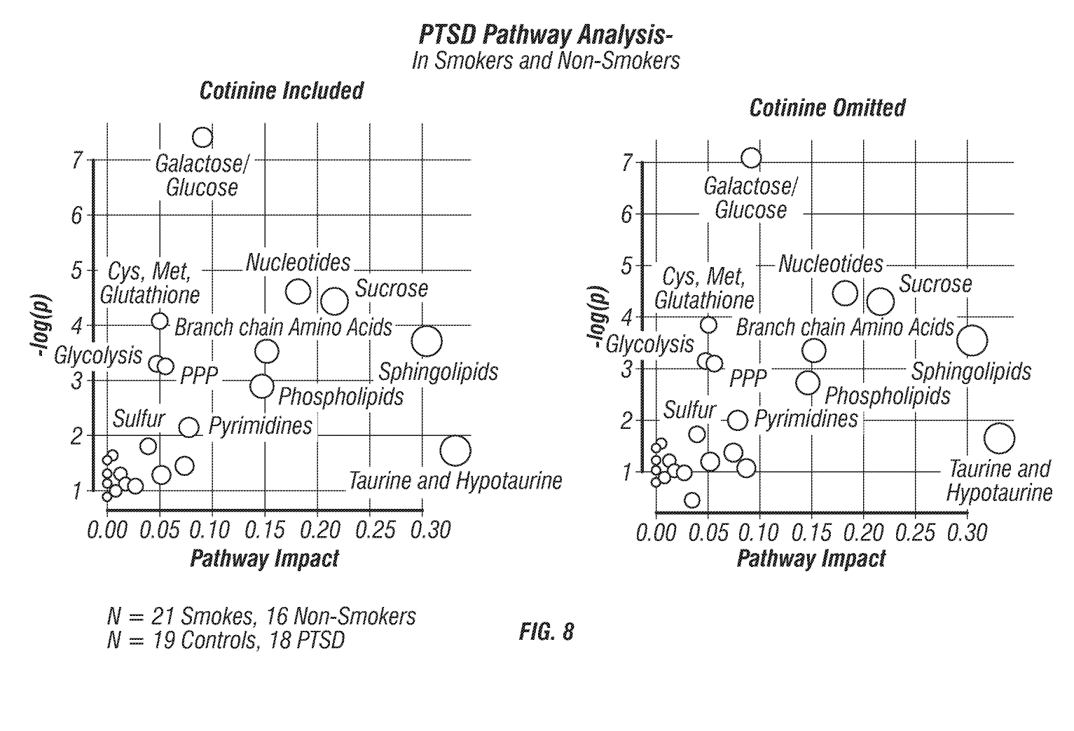

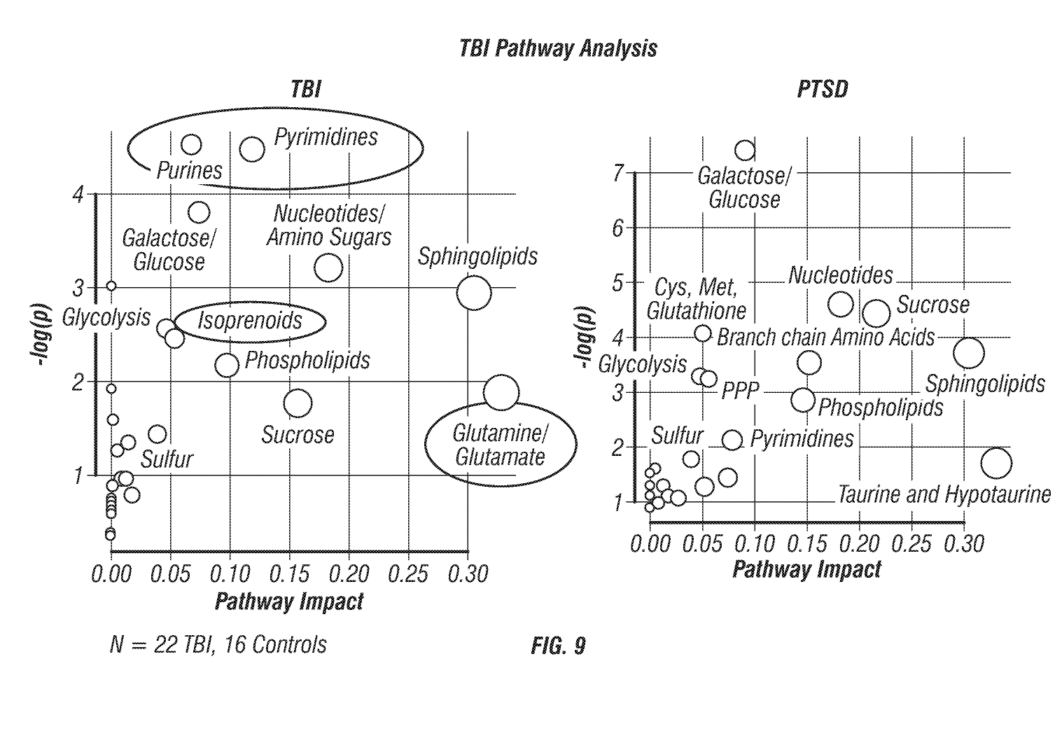

The metabolomic effects were measured in serum obtained from the subjects (20 control and 18 with PTSD). 475 metabolites were measured from 30 pathways by mass spectrometry (Table 2C), the data was analyzed by partial least squares discriminant analysis (PLSDA), and visualized by projection in three dimensions FIG. 1 and ranked by VIP scores FIG. 4. FIG. 1 shows that the top 20 metabolites (i.e., metabolites 1-20 in Table 2B) were sufficient to identify subjects with PTSD. FIG. 8 shows a depiction of metabolic pathways in PTSD smoker and non-smokers.

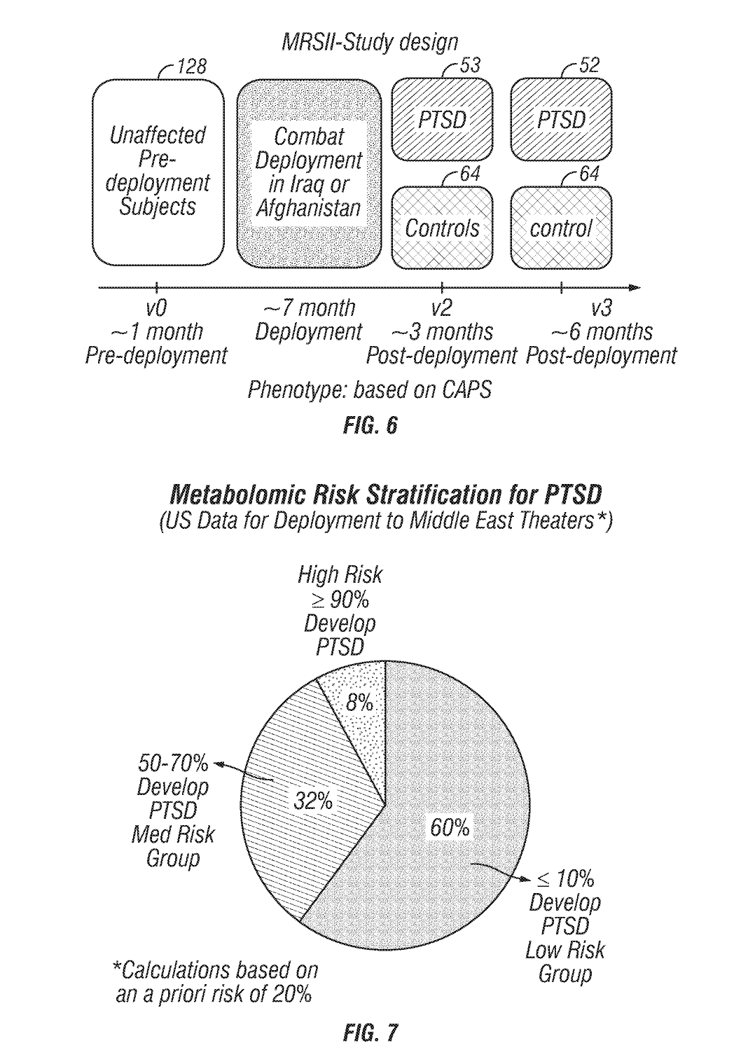

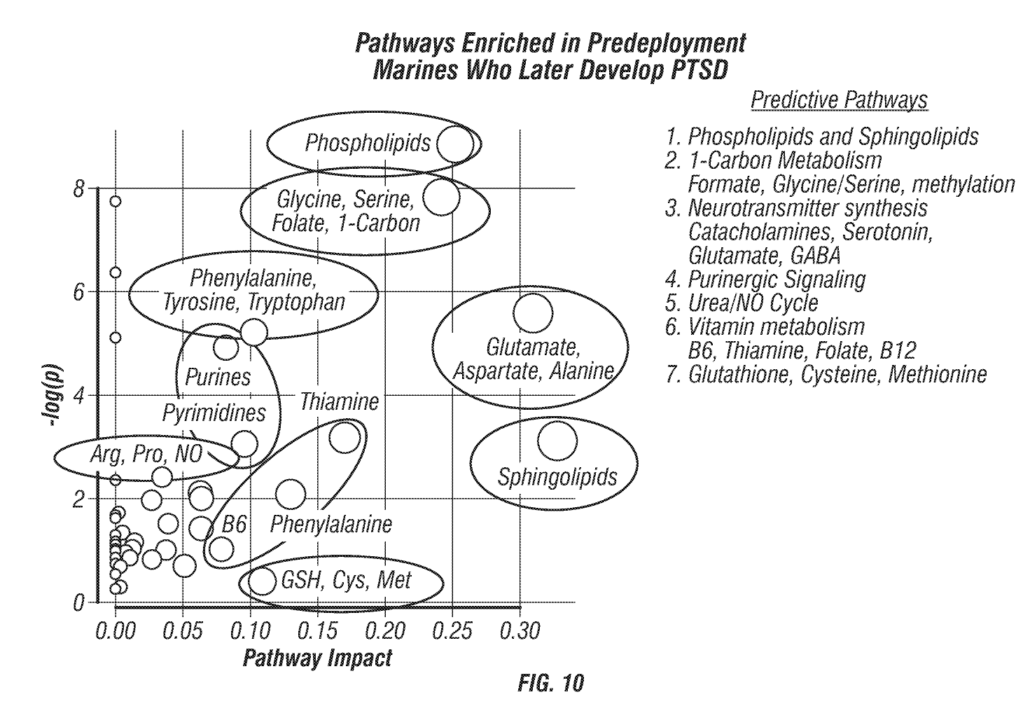

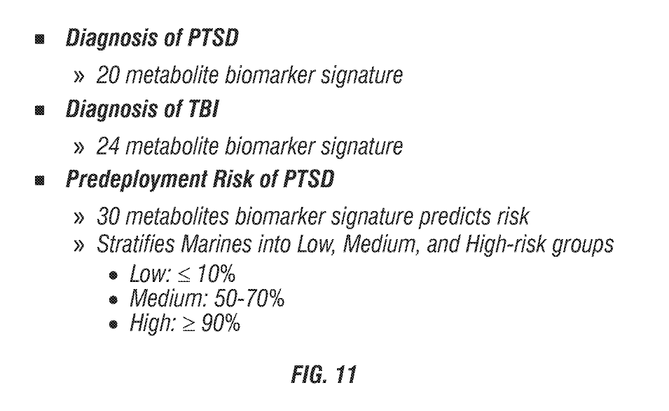

In addition, metabolomics experiments were performed to assess the risk of developing PTSD. In these experiments samples were obtained from subjects prior to a soldier deployment and the 475 metabolites measured from 30 pathways by mass spectrometry (Table 2C). The subject were then monitored for PTSD development by clinical manifestations of symptoms (see, FIG. 6). The metabolites were then analyzed by partial least squares discriminant analysis (PLSDA), and visualized by projection in three dimensions FIG. 2 (see also FIG. 7). As shown in FIG. 2, 30 metabolites were predictive of PTSD developmental risk. Metabolites from 7 pathways were predictive of PTSD risk. The metabolic pathways included (i) phospholipids and sphingolipids, (ii) 1-carbon metabolism (formate, glycine/serine, methylation), (iii) neurotransmitter synthesis (catacholamine, serotonin, glutamate, GABA), (iv) purinergic signaling, (v) urea/NO cycle, (vi) vitamin metabolism (vitamin B6, thiamine, folate, vitamin B12), and (vii) sulfur metabolic pathways (glutathione, cysteine, methionine) (see, FIG. 10). The metabolites analyzed were able to stratify soldiers into low, medium and high-risk groups (see, e.g., FIG. 11).

### Example 1B

Because traumatic brain injury (TBI) is related to aspect of PTSD, a metabolomics profile was performed on subjects with TBI.

TBI Metabolomics.

Broad spectrum analysis of 478 targeted metabolites from 44 biochemical pathways was performed. Samples were analyzed on an AB SCIEX QTRAP 5500 triple quadrupole mass spectrometer equipped with a Turbo V electrospray ionization (ESI) source, Shimadzu LC-20A UHPLC system, and a PAL CTC autosampler (AB ACIEX, Framingham, Mass., USA). Whole blood was collected into BD Microtainer tubes containing lithium heparin (Becton Dickinson, San Diego, Calif., USA, Ref#365971). Plasma was separated by centrifugation at 600 g×5 minutes at 20° C. within one hour of collection. Fresh lithium-heparin plasma was transferred to labeled tubes for storage at −80° C. for analysis. Typically 45 μl of plasma was thawed on ice and transferred to a 1.7 ml Eppendorf tube. Two and one-half (2.5) μl of a cocktail containing 35 commercial stable isotope internal standards, and 2.5 μl of 310 stable isotope internal standards that were custom-synthesized in E. coli and S. cerevisiae by metabolic labeling with ¹³C-glucose and ¹³C-bicarbonate, were added, mixed, and incubated for 10 min at room temperature to permit small molecules and vitamins in the internal standards to associate with plasma binding proteins. Macromolecules (protein, DNA, RNA, etc.) were precipitated by extraction with 4 volumes (200 μl) of cold (−20° C.), acetonitrile:methanol (50:50) (LCMS grade, Cat#LC015-2.5 and GC230-4, Burdick & Jackson, Honeywell), vortexed vigorously, and incubated on crushed ice for 10 min, then removed by centrifugation at 16,000 g×10 min at 4° C. The supernatants containing the extracted metabolites and internal standards in the resulting 40:40:20 solvent mix of acetonitrile:methanol:water were transferred to labeled cryotubes and stored at −80° C. for LC-MS/MS (liquid chromatography-tandem mass spectrometry) analysis.

LC-MS/MS analysis was performed by multiple reaction monitoring (MRM) under Analyst v1.6.1 (AB SCIEX, Framingham, Mass., USA) software control in both negative and positive mode with rapid polarity switching (50 ms). Nitrogen was used for curtain gas (set to 30), collision gas (set to high), ion source gas 1 and 2 (set to 35). The source temperature was 500° C. Spray voltage was set to −4500 V in negative mode and 5500 V in positive mode. The values for Q1 and Q3 mass-to-charge ratios (m/z), declustering potential (DP), entrance potential (EP), collision energy (CE), and collision cell exit potential (CXP) were determined and optimized for each MRM for each metabolite. Ten microliters of extract was injected by PAL CTC autosampler into a 250 mm×2 mm, 5 μm Luna NH₂ aminopropyl HPLC column (Phenomenex, Torrance, Calif., USA) held at 25° C. for chromatographic separation. The mobile phase was solvent A: 95% water with 23.18 mM NH₄OH (Sigma-Aldrich, St. Louis, Mo., USA, Fluka Cat#17837-100ML), 20 mM formic acid (Sigma, Fluka Cat#09676-100ML) and 5% acetonitrile (pH 9.44); solvent B: 100% acetonitrile. Separation was achieved using the following gradient: 0 min-95% B, 3 min-95% B, 3.1 min 80% B, 6 min 80% B, 6.1 min 70% B, 10 min 70% B, 18 min 2% B, 27 min 0% B, 32 min 0% B, 33 min 100% B, 36.1 95% B, 40 min 95% B end. The flow rate was 300 μl/min. All the samples were kept at 4° C. during analysis. The chromatographic peaks were identified using MultiQuant (v3.0, AB SCIEX), confirmed by manual inspection, and the peak areas integrated. The median of the peak area of stable isotope internal standards was calculated and used for the normalization of metabolites concentration across the samples and batches. Prior to multivariate and univariate analysis, the data were log-transformed.

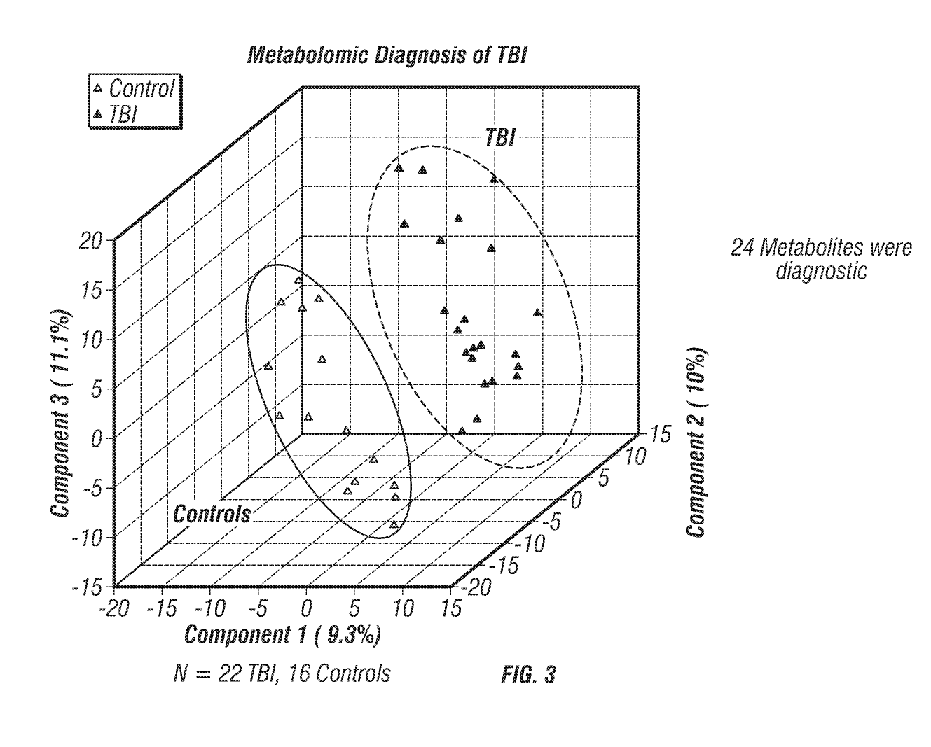

The metabolomic effects were measured in serum obtained from subjects (22 TBI subjects and 16 controls). 478 to 741 metabolites were measured from 17-44 pathways (see, e.g., Table 3) by mass spectrometry, the data was analyzed by partial least squares discriminant analysis (PLSDA), and the results visualized by projection in three dimensions FIG. 3. As shown in FIG. 3, 24 metabolites were diagnostic of TBI.

TABLE 3
Metabolic pathways in TBI:

| | Measured | | | | | | Fraction of |
|---|---|---|---|---|---|---|---|
| | Metabolites | Expected | Expected | Observed | | Impact | Impact |
| | in the | Pathway | Hits in | Hits in the | Fold | (Sum | (VIP) |
| | Pathway | Proportion | Sample of | Top 48 | Enrichment | VIP | Explained |
| Pathway Name | (N) | (P = N/477) | 48 (P * 48) | Metabolites | (Obs/Exp) | Score) | (%) |
| Purine Metabolism | 84 | 0.18 | 8.45 | 6 | 0.71 | 14.77 | 12.5% |
| Sphingolipid Metabolism | 77 | 0.16 | 7.75 | 6 | 0.77 | 14.49 | 12.2% |
| Phospholipid Metabolism | 214 | 0.45 | 21.53 | 6 | 0.28 | 14.06 | 11.9% |
| Pyrimidine Metabolism | 64 | 0.13 | 6.44 | 4 | 0.62 | 10.81 | 9.1% |
| Cholesterol, Cortisol, Non-Gonadal Steroid | 37 | 0.08 | 3.72 | 4 | 1.07 | 10.28 | 8.7% |
| Metabolism | | | | | | | |
| Glycolysis and Gluconeogenesis | 34 | 0.07 | 3.42 | 3 | 0.88 | 8.86 | 7.5% |
| Metabolism | | | | | | | |
| Amino-Sugar, Galactose, & Non-Glucose | 18 | 0.04 | 1.81 | 4 | 2.21 | 8.68 | 7.3% |
| Metabolism | | | | | | | |
| SAM, SAH, Methionine, Cysteine, Glutathione | 38 | 0.08 | 3.82 | 3 | 0.78 | 6.23 | 5.3% |
| Metabolism | | | | | | | |
| Microbiome Metabolism | 72 | 0.15 | 7.25 | 2 | 0.28 | 6.01 | 5.1% |
| Tryptophan, Kynurenine, Serotonin, Melatonin | 15 | 0.03 | 1.51 | 2 | 1.33 | 5.22 | 4.4% |
| Metabolism | | | | | | | |
| Bile Salt Metabolism | 8 | 0.02 | 0.81 | 1 | 1.24 | 4.04 | 3.4% |
| Pentose Phosphate, Gluconate Metabolism | 18 | 0.04 | 1.81 | 2 | 1.10 | 3.56 | 3.0% |
| Vitamin B2 (Riboflavin) Metabolism | 7 | 0.01 | 0.70 | 1 | 1.42 | 2.84 | 2.4% |
| Biopterin, Neopterin, Molybdopterin Metabolism | 2 | 0.00 | 0.20 | 1 | 4.97 | 2.24 | 1.9% |
| Phosphate and Pyrophosphate Metabolism | 1 | 0.00 | 0.10 | 1 | 9.94 | 2.17 | 1.8% |
| Bioamines and Neurotransmitter Metabolism | 23 | 0.05 | 2.31 | 1 | 0.43 | 2.09 | 1.8% |
| Krebs Cycle | 29 | 0.06 | 2.92 | 1 | 0.34 | 2.05 | 1.7% |

### Example 2

### Fragile X Model

Mouse Strain.

A Fragile X (Fmr1) knockout mouse was used on the FVB strain background. It has the genotype: FVB.129P2-Pde6b⁺ Tyr^{c-ch} Fmr1^{tm1Cgr}/J (Jackson Stock #004624). The Fmr1^{tm1Cgr} allele contains a neomycin resistance cassette replacing exon 5 that results in a null allele that makes no FMR mRNA or protein. The control strain used has the genotype: FVB.129P2-Pde6b⁺ Tyr^{c-ch}/AntJ (Jackson Stock #004828). In contrast to the white coat color of wild-type FVB mice, these animals had a chinchilla (Tyr^{c-ch}) gray coat color. The wild-type Pde6b locus from the 129P2 ES cells corrects the retinal degeneration phenotype that produces blindness by 5 weeks of age in typical FVB mice. The Fmr1 locus is X-linked, so males are hemizygous and females are homozygous for the knockout. A metabolomic analysis on Fmr1 knockout mice on the C57BL/6J background was also performed to refine the understanding of which metabolic disturbances were directly related to the Fmr1 knockout, and which were the result of changes in genetic background. For these studies the same Fmr1^{tm1Cgr} knockout allele bred on the C57BL6/J background was used. These animals had the genotype: B6.129P2-Fmr1^{tm1Cgr}/J (Jackson Stock#003025). The standard C57BL6/J strain (Jackson Stock#000664) was used as a control for the B6 metabolic studies.

The absence of Fragile X mental retardation protein (FMRP) expression in Fmr1 knockout mice, and its presence in FVB and C57BL/6J controls was confirmed by Western blot analysis before phenotyping the Fmr1 knockout animals used in this study.

Animal Husbandry and Care.

All studies were conducted in facilities accredited by the Association for Assessment and Accreditation of Laboratory Animal Care International (AAALAC), and followed the National Institutes of Health (NIH) Guidelines for the use of animals in research. Five-week old male mice were obtained from Jackson Laboratories (Bar Harbor, Me.), identified by ear tags, placed in cages of 2-4 animals, and maintained on ad libitum Harlan Teklad 8604 mouse chow (14% fat, 54% carbohydrate, 32% protein) and water. Animals were housed in a temperature (22-24° C.) and humidity (40-55%) controlled vivarium with a 12 h light-dark cycle (lights on at 7 AM). No mice were housed in isolation. Beginning at 9 weeks of age, animals received weekly injections of either saline (5 μl/g ip) or suramin (hexasodium salt, 20 mg/kg ip; Tocris Cat #1472).

Behavioral Testing.

Behavioral testing began at 13 weeks of age, after one month of weekly antipurinergic therapy with suramin. Mice were tested in social approach, T-maze, locomomtor activity, marble burying, acoustic startle, and prepulse inhibition paradigms as follows.

Social Preference and Social Novelty.

Social behavior was tested as social preference described in Example 3, with the addition of a third phase with a second novel mouse to interrogate social novelty.

Altered social behavior is a measure of autism-like features in mouse models of autism. In the Fragile X knockout genetic model of autism, it has also been a reproducible paradigm across different studies (Budimirovic and Kaufmann, 2011). Males with the Fragile X knockout showed a 26% reduction in social preference, as measured by the time spent interacting with a stranger mouse compared to an inanimate object. There was also a 35% reduction in social novelty, as measured by the time spent interacting with a novel mouse compared to a familiar mouse. This altered social behavior was corrected by antipurinergic therapy with suramin.

T-Maze.

Novelty preference was tested as spontaneous alternation behavior in the T-maze as described in Example 3.

Novelty preference is an innate feature of normal rodent (Hughes, 2007) and human (Vecera et al., 1991) behavior, and a predictor of socialization and communication growth in children with ASD (Munson et al., 2008). The loss or suppression of novelty preference in children with autism spectrum disorders (ASD) is associated with the phenomenon known as insistence on sameness (Gotham et al., 2013). A preference for novelty was estimated as spontaneous alternation behavior in the T-maze. The T-maze can also be used to estimate spatial working memory, especially when food motivated. The Fragile X knockout mice showed deficient novelty preference as reflected by chance (near 50%) spontaneous alternation behavior. These deficits were normalized by suramin treatment. Fragile X knockout mice were no different from controls in latency to choice.

Marble Burying.

Marble burying behavior was measured over 30 minutes by a modification of methods used by Thomas, et al. (Thomas et al., 2009).

Marble burying was used as a measure of normal rodent digging behavior. Marble burying has sometimes been considered a measure of anxiety, however, comprehensive genetic and behavioral studies have shown that marble burying is a normal mouse behavior that is genetically determined (Thomas et al., 2009). Marble burying was diminished 38% in Fragile X knockout mice. Suramin improved this (KO-Sal v KO-Sur).

Locomotor Activity.

Locomotor activity, hyperactivity (total distance traveled), center entries, holepoke exploration, and vertical investigation (rearing) behaviors were quantified by automated beam break analysis in the mouse behavioral pattern monitor (mBPM) as previously described (Halberstadt et al., 2009).

Acoustic Startle and Prepulse Inhibition.

Sensitivity to acoustic startle and prepulse inhibition of the startle reflex were measured by automated testing in commercial startle chambers as previously described (Asp et al., 2010).

Body Temperature Measurements.

A BAT-12 Microprobe digital thermometer and RET-3 mouse rectal probe (Physitemp Instruments, Clifton, N.J.) were used to obtain rectal core temperatures to a precision of +/−0.1° C. Care was taken to measure temperatures ≧2 days after cage bedding changes, and to avoid animal transport stress immediately prior to measurement in order to avoid stress-induced hyperthermia (Adriaan Bouwknecht et al., 2007). Temperatures were measured between 9 am to 12 noon each day.

Fmr1 knockout mice showed relative hypothermia of about 0.5-0.7° C. below the basal body temperature of the FVB controls. The maternal immune activation (MIA) mouse model showed a similar mild reduction in body temperature that was consistent with pathologic persistence of the cell danger response. Normal basal body temperature was restored by antipurinergic therapy with suramin. Suramin had no effect on the body temperature of control animals (WT-Sal vs WT-Sur).

Synaptosome Isolation and Ultrastructure.

Cerebral samples were collected, homogenized and synaptosomes isolated by discontinuous Percoll gradient centrifugation, drop dialyzed, glutaraldehyde fixed, post-fixed in osmium tetroxide, embedded, sectioned, and stained with uranyl acetate for transmission electron microscopy. Samples from the FVB control animals (+/−suramin) were not available for study by either electron microscopy or Western analysis. Therefore, only the effects of suramin on the two groups of FMR knockout animals (KO-saline and KO-suramin) are provided.

Studies showed synaptic ultrastructural abnormalities in the maternal immune activation (MIA) mouse model that were corrected by antipurinergic therapy. In that study, in which neuroinflammation and the cell danger response (CDR) play a role in pathogenesis, the animals with ASD-like behaviors were found to have abnormal synaptosomes containing an electron dense matrix and brittle or fragile and hypomorphic post-synaptic densities. In the present study of the Fragile X model, saline-treated Fmr1 knockout mice had cerebral synaptosomes that also contained an electron dense matrix, and fragile, hypomorphic post-synaptic densities. In contrast, suramin-treated mice had near-normal appearing cerebral synaptosomes with an electron lucent matrix and normal appearing post-synaptic densities.

17 of 54 proteins that were interrogated in cerebral synaptosomes (See table 4) were changed by antipurinergic therapy with suramin in the Fragile X model. As a treatment study, focus was placed on the effect of suramin in the Fmr1 knockout mice only. The current study did not compare knockout brain protein levels to littermate FVB controls.

The PI3/AKT/GSK3β pathway is pathologically elevated in the Fragile X model. Suramin inhibited this pathway at several points. Suramin decreased the expression of PI3 Kinase and AKT, and increased the inhibitory phosphorylation of the PI3K/AKT pathway proteins glycogen synthase kinase 3β (GSK3β) by 75%, and S6 kinase (S6K) by 47%. A corresponding change in mTOR expression or phosphorylation was not observed in this model (Table 4).

Adenomatous polyposis coli (APC) is a tumor suppressor protein that is increased in the Fragile X knockout model. APC forms a complex with, and is phosphorylated by, active GSK3β to inhibit microtubule assembly during undifferentiated cell growth of neuronal progenitors (Arevalo and Chao, 2005). Suramin treatment returned total APC to normal by decreasing expression by 29%.

Chronic hyperpurinergia associates with the MIA mouse model and results in downregulation of expression of the P2Y2 receptor. Suramin treatment in the MIA model increased P2Y2 expression to normal levels. In the Fragile X mouse model, suramin treatment increased the expression of the P2Y1 receptor 31%, and decreased P2X3 receptor expression 18%. There was no effect on P2Y2 expression (Table 4). P2Y1 signaling is known to inhibit IP3 gated calcium release from the endoplasmic reticulum. Suramin treatment was associated with a 101% increase in IP3R1 expression.

AMPA receptor (GluR1) mRNA transcription, translation, and receptor recycling are known to be pathologically dysregulated in the Fragile X model. Suramin treatment decreased the overall expression of the ionotropic GluR1 by 15% but had no effect on metabotropic glutamate receptor mGluR5 expression (Table 4).

Cannabinoid signaling is pathologically increased in the FMR knockout model. Suramin treatment decreased CB1 receptor expression 16%. This is consistent with recent data that has shown endocannabinoid signaling to be sharply increased in response to the cell danger response (CDR) produced by brain injury. Pharmacologic blockade with the CB1R antagonist rimonabant has been shown to improve several symptoms in the Fragile X model. CB2 expression is increased in the peripheral blood monocytes of children with autism spectrum disorders. However, CB2 receptor expression in the brain synaptosomes of the Fragile X model was unchanged (Table 4).

PPARβ (also known as PPARδ) is a widely expressed transcriptional coactivator that is correlated with the aerobic and bioenergetic capacity in a variety of tissue types. Suramin treatment increased the expression of PPARβ in purified brain synaptosomes by 34%. Suramin treatment had no effect on synaptosomal PPARα (Table 4).

Antipurinergic therapy with suramin increased three key proteins involved in sterol and bile acid synthesis. 7-dehydrocholesterol reductase (7DHCR) was increased by 24%, cholesterol 7α-hydroxylase (CYP7A1) by 37%, steroidogenic acute regulatory (StAR) protein by 165%. The function of bile salts in the brain is unknown, although their neuroprotective effects have been documented in several models.

Recent studies have revealed an important role for complement proteins in tagging synapses during inflammation and remodeling. Activated complement proteins have also been found in the brains of children with autism. Suramin decreased synaptosomal C1qA by 24%.

Tar-DNA binding protein 43 (TDP43) is and single-strand DNA and RNA binding protein that disturbs mitochondrial transport and function under conditions of cell stress. Mutations in TDP43 are associated with genetic forms of amyotrophic lateral sclerosis (ALS). Wild-type TDP43 protein is a component of the tau and α-synuclein inclusion bodies found in Alzheimer and Parkinson disease and plays a role in RNA homeostasis and protein translation. The similarities of these functions to the role of the Fmr1 gene in RNA homeostasis prompted investigation of TDP43 in the Fragile X model. Suramin treatment decreased synaptosomal TDP43 by 27%.

A number of recent papers have identified the upregulation of gene networks in ASD and inborn errors of purine metabolism that were formerly thought to be specific for Alzheimer and other neurodegenerative disorders. Amyloid-β precursor protein (APP) expression is upregulated in the brain of subjects with ASD. Antipurinergic therapy with suramin decreased synaptosomal APP levels by 23% in the Fragile X model.

The effect of suramin on several additional proteins that were found to be dysregulated in the MIA mouse model were also examined. No effect of suramin in the Fragile X model on ERK 1 and 2, or its phosphorylation, CAMKII or its phosphorylation, nicotinic acetylcholine receptor alpha 7 subunit (nAchRα7) expression, or the expression of the purinergic receptors P2Y2 and P2X7 were observed (Table 4). These data show that the detailed molecular effects of antipurinergic therapy with suramin are different in different genetic backgrounds and different mechanistic models of autism spectrum disorders. However, the efficacy in restoring normal behavior and brain synaptic morphology cuts across models. These data support the conclusion that antipurinergic therapy is operating by a metabolic mechanism that is common to, and underlies, both the environmental MIA, and the genetic Fragile X models of ASD.

Western Blot Analysis.

Twenty μg of cerebral synaptosomal protein was loaded in SDS-polyacrylamide gels (Bis-Tris Gels) and transferred to PVDF membranes. The blots were first stained with Ponceau S, scanned, and the transfer efficiency was quantified by densitometry before blocking with 3% skim milk, and probing with primary and secondary antibodies for signal development by enhanced chemiluminescence (ECL). The cerebral synaptosome expression of 54 proteins was evaluated (Table 4).

TABLE 4

| | | Response to Suramin | | | |
|---|---|---|---|---|---|
| No. | Protein/Antibody Target | MW (KDa) | KO-Sur/KO-Sal | Vendor | Cat# |
| 1 | PI3K | 100 | Down | Cell Signaling | #3811 |
| 2 | Akt | 60 | Down | Cell Signaling | #9272 |
| 3 | pGSK3β (Ser9) | 50 | Up | Cell Signaling | #9323 |
| 4 | pS6K(Thr389) | 70 | Up | Cell signaling | #9205 |
| 5 | APC | 310 | Down | Cellsignaling | #2504 |
| 6 | P2Y1R | 48 | Up | Alomone Labs | #APR-009 |
| 7 | P2X3R | 44 | Down | Alomone Labs | #APR-026 |
| 8 | IP3R I | 320 | Up | Cellsignaling | #3763 |
| 9 | GluR1 | 106 | Down | Abcam | #ab172971 |
| 10 | CB1 | 53 | Down | Abcam | #ab172970 |
| 11 | PPAR beta/delta | 50 | Up | Abcam | #ab23673 |
| 12 | 7-dehydrocholesterol reductase/7DHCR | 54 | Up | Abcam | #ab103296 |
| 13 | Cholesterol 7 alpha-hydroxylase/CYP7A1 | 55 | Up | Abcam | #ab65596 |
| 14 | Steroidogenic acute regulatory protein/StAR | 50 | Up | Cell Signaling | #8449 |
| 15 | C1qA | 25 | Down | Abcam | #ab155052 |
| 16 | TAR DNA-binding protein 43/TDP43 | 45 | Down | Cell Signaling | #3449 |
| 17 | Amyloid β (Aβ) precursor protein/APP | 100-140 | Down | Cellsignaling | #2452 |
| 18 | pCAMKII(Thr286) | 50, 60 | None | Cellsignaling | #3361 |
| 19 | pERK1/2(Thr202/Tyr204) | 42, 44 | None | Cell Signaling | #4370 |
| 20 | pSTAT3(ser727) | 86 | None | Cell Signaling | #9134 |
| 21 | P2Y2 | 42 | None | Alomone Labs | #APR-010 |
| 22 | P2Y4 | 41 | None | Alomone Labs | #APR-006 |
| 23 | P2X1 | 45 | None | Alomone Labs | #APR-022 |
| 24 | P2X2 | 44 | None | Alomone Labs | #APR-025 |
| 25 | P2X4 | 43 | None | Alomone Labs | #APR-024 |
| 26 | P2X5 | 47 | None | Alomone Labs | #APR-005 |
| 27 | P2X6 | 50 | None | Alomone Labs | #APR-013 |
| 28 | P2X7 | 68 | None | Alomone Labs | #APR-004 |
| 29 | Metabotropic glutamate receptor 5/mGluR5 | 132 | None | Abcam | #ab76316 |
| 30 | Nicotinic Acetylcholine Receptor alpha 7/nAchR7 [symbol] | 50 | None | Abcam | #ab23832 |
| 31 | GABA A Receptor beta 3/GABA-β3 | 54 | None | Abcam | #ab4046 |
| 32 | Dopamine Receptor D4/D4R | 42 | None | Alomone Labs | #ADR-004 |
| 33 | ETFQO/ETFDH | 65 | None | Abcam | #ab126576 |
| 34 | Methionine Sulfoxide Reductase A/MSRA | 30 | None | Abcam | #ab16803 |
| 35 | Acetyl-CoA acetyltransferase 2/ACAT2 | 41 | None | Cellsignal | #11814 |
| 36 | HMGCoA Reductase/HMOCoAR | 97 | None | BioVision | #3952-100 |
| 37 | Indoleamine 2,3-dioxygenase 1/IDO-1 | 45 | None | Millipore | #MAB5412 |
| 38 | p-mTOR(ser2448) | 289 | None | Cell Signaling | #2971 |
| 39 | mTOR | 289 | None | Cell Signaling | #2972 |
| 40 | pPERK(Thr980) | 170 | None | Cell Signaling | #3179 |
| 41 | p-eIF2α(Ser51) | 38 | None | Cell Signaling | #9721 |
| 42 | Nitro Tyrosine | 10-200 | None | Abcam | #ab7048 |
| 43 | TGFβ Receptor I | 50 | None | Abcam | #ab31013 |
| 44 | CB2 | 45 | None | Abcam | ab45942 |
| 45 | PGC1a | 115 | None | Abcam | #ab54481 |
| 46 | PPARa | 53 | None | Santa Cruz | #sc-9000 |
| 47 | CPY27A1 | 60 | None | Abcam | #ab151987 |
| 48 | pAkt(Thr308) | 60 | None | Cell Signaling | #4056 |
| 49 | pAkt(Ser473) | 60 | None | Cell Signaling | #9018 |
| 50 | PKC | 82 | None | Abcam | #ab19031 |
| 51 | pPKC(Ser660) | 80 | None | Cell Signaling | #9371 |
| 52 | nAchR beta2 | 70 | None | Alomone Labs | #ANC-012 |
| 53 | Postsynaptic Density protein 95/PSD95 | 95 | None | Cell Signaling | #3450 |
| 54 | Fragile X mental retardation protein/FMRP | 80 | None | Cell Signaling | #4317 |

[symbol]

indicates data missing or illegible when filed

Metabolomics.

Broad spectrum analysis of 673 targeted metabolites from 60 biochemical pathways was performed. Samples were analyzed on an AB SCIEX QTRAP 5500 triple quadrupole mass spectrometer equipped with a Turbo V electrospray ionization (ESI) source, Shimadzu LC-20A UHPLC system, and a PAL CTC autosampler (AB ACIEX, Framingham, Mass., USA). Whole blood was collected 3-4 days after the last weekly dose of suramin (20 mg/kg ip) or saline (5 μl/g ip), after light anesthesia in an isoflurane (Med-Vet International, Mettawa, Ill., USA, Cat#RXISO-250) drop jar, into BD Microtainer tubes containing lithium heparin (Becton Dickinson, San Diego, Calif., USA, Ref#365971) by submandibular vein lancet (Golde et al., 2005). Plasma was separated by centrifugation at 600 g×5 minutes at 20° C. within one hour of collection. Fresh lithium-heparin plasma was transferred to labeled tubes for storage at −80° C. for analysis. Typically 45 μl of plasma was thawed on ice and transferred to a 1.7 ml Eppendorf tube. Two and one-half (2.5) μl of a cocktail containing 35 commercial stable isotope internal standards, and 2.5 μl of 310 stable isotope internal standards that were custom-synthesized in E. coli and S. cerevisiae by metabolic labeling with ¹³C-glucose and ¹³C-bicarbonate, were added, mixed, and incubated for 10 min at room temperature to permit small molecules and vitamins in the internal standards to associate with plasma binding proteins. Macromolecules (protein, DNA, RNA, etc.) were precipitated by extraction with 4 volumes (200 μl) of cold (−20° C.), acetonitrile:methanol (50:50) (LCMS grade, Cat#LC015-2.5 and GC230-4, Burdick & Jackson, Honeywell), vortexed vigorously, and incubated on crushed ice for 10 min, then removed by centrifugation at 16,000 g×10 min at 4° C. The supernatants containing the extracted metabolites and internal standards in the resulting 40:40:20 solvent mix of acetonitrile:methanol:water were transferred to labeled cryotubes and stored at −80° C. for LC-MS/MS (liquid chromatography-tandem mass spectrometry) analysis.

LC-MS/MS analysis was performed by multiple reaction monitoring (MRM) under Analyst v1.6.1 (AB SCIEX, Framingham, Mass., USA) software control in both negative and positive mode with rapid polarity switching (50 ms). Nitrogen was used for curtain gas (set to 30), collision gas (set to high), ion source gas 1 and 2 (set to 35). The source temperature was 500° C. Spray voltage was set to −4500 V in negative mode and 5500 V in positive mode. The values for Q1 and Q3 mass-to-charge ratios (m/z), declustering potential (DP), entrance potential (EP), collision energy (CE), and collision cell exit potential (CXP) were determined and optimized for each MRM for each metabolite. Ten microliters of extract was injected by PAL CTC autosampler into a 250 mm×2 mm, 5 μm Luna NH₂ aminopropyl HPLC column (Phenomenex, Torrance, Calif., USA) held at 25° C. for chromatographic separation. The mobile phase was solvent A: 95% water with 23.18 mM NH₄OH (Sigma-Aldrich, St. Louis, Mo., USA, Fluka Cat#17837-100ML), 20 mM formic acid (Sigma, Fluka Cat#09676-100ML) and 5% acetonitrile (pH 9.44); solvent B: 100% acetonitrile. Separation was achieved using the following gradient: 0 min-95% B, 3 min-95% B, 3.1 min 80% B, 6 min 80% B, 6.1 min 70% B, 10 min 70% B, 18 min 2% B, 27 min 0% B, 32 min 0% B, 33 min 100% B, 36.1 95% B, 40 min 95% B end. The flow rate was 300 μl/min. All the samples were kept at 4° C. during analysis. The chromatographic peaks were identified using MultiQuant (v3.0, AB SCIEX), confirmed by manual inspection, and the peak areas integrated. The median of the peak area of stable isotope internal standards was calculated and used for the normalization of metabolites concentration across the samples and batches. Prior to multivariate and univariate analysis, the data were log-transformed.

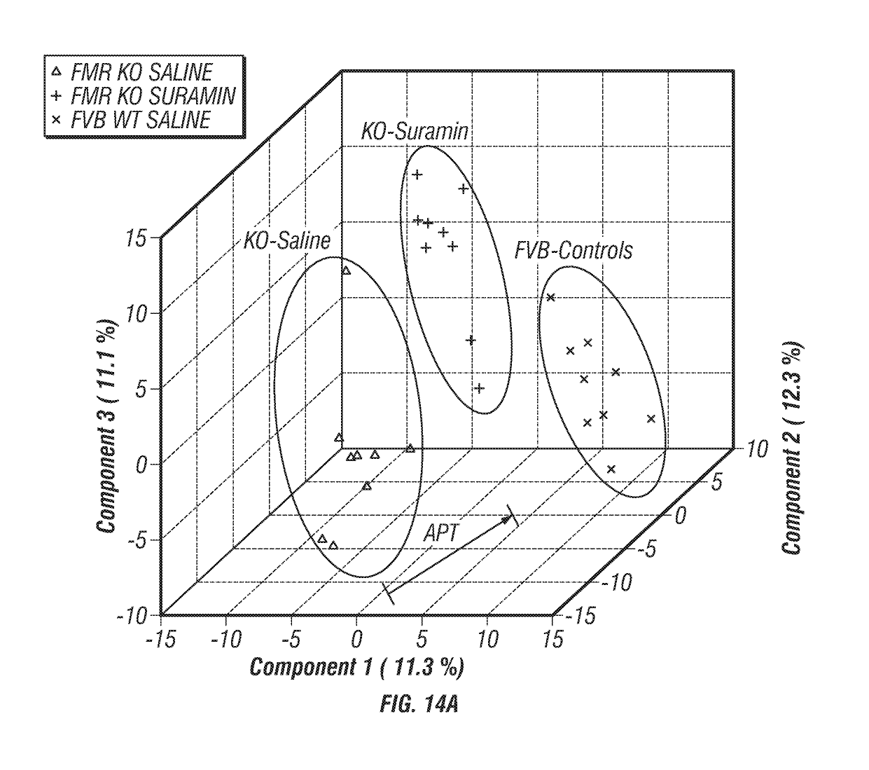

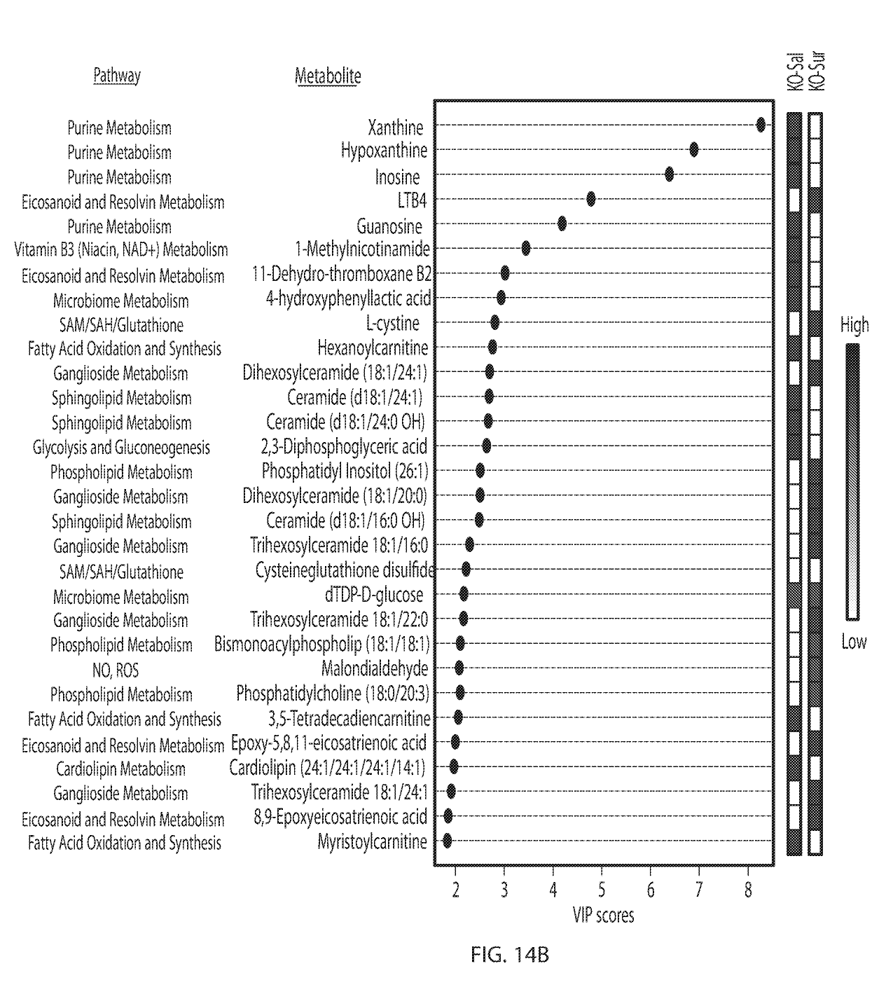

The metabolomic effects were measured in plasma after weekly treatment with suramin or saline. 673 metabolites were measured from 60 pathways by mass spectrometry (Table 5), analyzed the data by partial least squares discriminant analysis (PLSDA), and visualized the results by projection in three dimensions FIG. 14A, and ranked by VIP scores FIG. 14B. Suramin produced pharmacometabolomic changes in one third of the biochemical pathways interrogated (20 of 60 pathways).

TABLE 5

| No. | Pathway | Metabolites |
|---|---|---|
| 1 | 1-Carbon, Folate, Formate, Glycine, Serine | 9 |
| | Metabolism | |
| 2 | Amino Acid Metabolism (not otherwise covered) | 4 |
| 3 | Amino-Sugar, Galactose, & Non-Glucose | 10 |
| | Metabolism | |
| 4 | Bile Salt Metabolism | 8 |
| 5 | Bioamines and Neurotransmitter Metabolism | 11 |
| 6 | Biopterin, Neopterin, Molybdopterin Metabolism | 2 |
| 7 | Biotin (Vitamin B7) Metabolism | 1 |
| 8 | Branch Chain Amino Acid Metabolism | 13 |
| 9 | Cardiolipin Metabolism | 12 |
| 10 | Cholesterol, Cortisol, Non-Gonadal Steroid | 29 |
| | Metabolism | |
| 11 | Eicosanoid and Resolvin Metabolism | 36 |
| 12 | Endocannabinoid Metabolism | 2 |
| 13 | Fatty Acid Oxidation and Synthesis | 39 |
| 14 | Food Sources, Additives, Preservatives, Colorings, | 3 |
| | and Dyes | |
| 15 | Forensic Drugs | 1 |
| 16 | GABA, Glutamate, Arginine, Ornithine, Proline | 6 |
| | Metabolism | |
| 17 | Gamma-Glutamyl and other Dipeptides | 6 |
| 18 | Ganglioside Metabolism | 12 |
| 19 | Glycolysis and Gluconeogenesis Metabolism | 18 |
| 20 | Gonadal Steroids | 2 |
| 21 | Heme and Porphyrin Metabolism | 4 |
| 22 | Histidine, Histamine, Carnosine Metabolism | 5 |
| 23 | Isoleucine, Valine, Threonine, or Methionine | 4 |
| | Metabolism | |
| 24 | Ketone Body Metabolism | 2 |
| 25 | Krebs Cycle | 17 |
| 26 | Lysine Metabolism | 3 |
| 27 | Microbiome Metabolism | 33 |
| 28 | Nitric Oxide, Superoxide, Peroxide Metabolism | 6 |
| 29 | OTC and Prescription Pharmaceutical Metabolism | 3 |
| 30 | Oxalate, Glyoxylate Metabolism | 3 |
| Subtotal | | 304 |
| TOTAL Pathways | | 60 |
| 31 | Pentose Phosphate, Gluconate Metabolism | 11 |
| 32 | Phosphate and Pyrophosphate Metabolism | 1 |
| 33 | Phospholipid Metabolism | 115 |
| 34 | Phytanic, Branch, Odd Chain Fatty Acid | 1 |
| | Metabolism | |
| 35 | Phytonutrients, Bioactive Botanical Metabolites | 3 |
| 36 | Plasmalogen Metabolism | 4 |
| 37 | Polyamine Metabolism | 6 |
| 38 | Purine Metabolism | 41 |
| 39 | Pyrimidine Metabolism | 31 |
| 40 | SAM, SAH, Methionine, Cysteine, Glutathione | 22 |
| | Metabolism | |
| 41 | Sphingolipid Metabolism | 72 |
| 42 | Taurine, Hypotaurine Metabolism | 2 |
| 43 | Thyroxine Metabolism | 1 |
| 44 | Triacylglycerol Metabolism | 1 |
| 45 | Tryptophan, Kynurenine, Serotonin, Melatonin | 10 |
| | Metabolism | |
| 46 | Tyrosine and Phenylalanine Metabolism | 4 |
| 47 | Ubiquinone and Dolichol Metabolism | 4 |
| 48 | Urea Cycle | 4 |
| 49 | Very Long Chain Fatty Acid Oxidation | 3 |
| 50 | Vitamin A (Retinol), Carotenoid Metabolism | 3 |
| 51 | Vitamin B1 (Thiamine) Metabolism | 3 |
| 52 | Vitamin B12 (Cobalamin) Metabolism | 3 |
| 53 | Vitamin B2 (Riboflavin) Metabolism | 4 |
| 54 | Vitamin B3 (Niacin, NAD+) Metabolism | 8 |
| 55 | Vitamin B5 (Pantothenate, CoA) Metabolism | 1 |
| 56 | Vitamin B6 (Pyridoxine) Metabolism | 5 |
| 57 | Vitamin C (Ascorbate) Metabolism | 2 |
| 58 | Vitamin D (Calciferol) Metabolism | 2 |
| 59 | Vitamin E (Tocopherol) Metabolism | 1 |
| 60 | Vitamin K (Menaquinone) Metabolism | 1 |

| | |
|---|---|
| Subtotal | 369 |
| TOTAL Metabolites | 673 |

The top 11 of 20 discriminating metabolic pathways were represented by 2 or more metabolites and explained 89% of the biochemical variance in the Fragile X mouse model treated with suramin (Table 6). These pathways were: purines (20%), fatty acid oxidation (12%), eicosanoids (11%), gangliosides (10%), phospholipids (9%), sphingolipids (8%), microbiome (5%), SAM/SAH glutathione (5%), NAD+ metabolism (4%), glycolysis (3%), and cholesterol metabolism (2%) (Table 6).

TABLE 6A
Biochemical Pathways with Metabolites Changed
by Antipurinergic Therapy in the Fragile X Model:

| | | Measured | Expected | Expected | Observed | | Impact | Fraction of | Suramin |
|---|---|---|---|---|---|---|---|---|---|
| | | Metabolites | Pathway | Hits in | Hits in | Fold | (Sum | Impact (VIP) | Treatment |
| | | in the | Proportion | Sample of 58 | the Top 58 | Enrichment | VIP | Explained | Effect (KO- |
| No. | Pathway Name | Pathway (N) | (P = N/673) | (P * 58) | Metabolites | (Obs/Exp) | Score) | (% of 136.0) | Sur/KO-Sal) |
| 1 | Purine Metabolism | 41 | 0.061 | 3.54 | 5 | 1.41 | 27.2 | 20.0% | 4/5 Decreased |
| 2 | Fatty Acid Oxidation and | 39 | 0.057 | 3.37 | 9 | 2.67 | 16.8 | 12.4% | 9/9 Decreased |
| | Synthesis | | | | | | | | |
| 3 | Eicosanoid and Resolvin | 36 | 0.053 | 3.11 | 6 | 1.93 | 14.7 | 10.8% | 4/6 Increased |
| | Metabolism | | | | | | | | |
| 4 | Ganglioside Metabolism | 12 | 0.018 | 1.04 | 6 | 5.79 | 13.4 | 9.8% | 6/6 Increased |
| 5 | Phospholipid Metabolism | 115 | 0.18 | 9.93 | 6 | 0.60 | 11.5 | 8.5% | 6/6 Increased |
| 6 | Sphingolipid Metabolism | 72 | 0.105 | 6.21 | 5 | 0.80 | 11.1 | 8.2% | 3/5 Decreased |
| 7 | Microbiome Metabolism | 33 | 0.047 | 2.85 | 3 | 1.05 | 6.7 | 4.9% | 2/3 Decreased |
| 8 | SAM, SAH, Methionine, | 22 | 0.032 | 1.90 | 3 | 1.58 | 6.7 | 4.9% | 3/3 Increased |
| | Cysteine, Glutathione | | | | | | | | |
| | Metabolism | | | | | | | | |
| 9 | Vitamin B3 (Niacin, NAD+) | 8 | 0.012 | 0.69 | 2 | 2.90 | 5.2 | 3.8% | 1/2 Increased |
| | Metabolism | | | | | | | | |
| 10 | Glycolysis and | 18 | 0.026 | 1.55 | 2 | 1.29 | 4.2 | 3.1% | 2/2 Decreased |
| | Gluconeogenesis | | | | | | | | |
| 11 | Cholesterol, Cortisol, | 29 | 0.042 | 2.50 | 2 | 0.80 | 3.2 | 2.4% | 2/2 Increased |
| | Non-Gonadal Steroid | | | | | | | | |
| | Metabolism | | | | | | | | |
| 12 | Nitric Oxide, Superoxide, | 6 | 0.009 | 0.52 | 1 | 1.93 | 2.1 | 1.5% | Increased |
| | Peroxide Metabolism | | | | | | | | |
| 13 | Cardiolipin Metabolism | 12 | 0.018 | 1.04 | 1 | 0.97 | 2.0 | 1.4% | Decreased |
| 14 | Bile Salt Metabolism | 8 | 0.012 | 0.69 | 1 | 1.45 | 1.8 | 1.3% | Increased |
| 15 | Branch Chain Amino Acid | 13 | 0.019 | 1.12 | 1 | 0.89 | 1.7 | 1.2% | Increased |
| | Metabolism | | | | | | | | |
| 16 | Isoleucine, Valine, Threonine, | 4 | 0.006 | 0.35 | 1 | 2.90 | 1.7 | 1.2% | Increased |
| | or Methionine Metabolism | | | | | | | | |
| 17 | Pyrimidine Metabolism | 31 | 0.051 | 2.68 | 1 | 0.37 | 1.6 | 1.1% | Decreased |
| 18 | Krebs Cycle | 17 | 0.025 | 1.47 | 1 | 0.68 | 1.6 | 1.1% | Increased |
| 19 | Vitamin B6 (Pyridoxine) | 5 | 0.007 | 0.43 | 1 | 2.32 | 1.5 | 1.1% | Increased |
| | Metabolism | | | | | | | | |
| 20 | Pentose Phosphate, Gluconate | 11 | 0.016 | 0.95 | 1 | 1.05 | 1.5 | 1.1% | Increased |
| | Metabolism | | | | | | | | |
| | 20 of 60 Pathways | 532 | 79% | 46 | 58 | | 136.0 | 100% | 33/58 Increased |
| | Dysregulated | (0.79 × 673) | (532/673) | (0.79 × 58) | | | | | |

Table 6A Legend.
Pathways were ranked by their impact measured by summed VIP (Σ_{VIP}; variable importance in projection) scores. A total of 58 metabolites were found to discriminate suramin-treated and saline-treated Fragile X knockout groups by multivariate partial least squares discriminant analysis (PLSDA). Significant metabolites had VIP scores of ≧1.5. Twenty (33%) of the 60 pathways interrogated had at least one metabolite with VIP scores ≧1.5. The total impact of these 58 metabolites corresponded to a summed VIP score of 136. The fractional impact of each pathway is quantified as the percent of the summed VIP score and displayed in the final column on the right in the table. Antipurinergic therapy with suramin not only corrected purine metabolism, but also produced changes in 19 other pathways associated with multi-system improvements in ASD-like symptoms.

TABLE 6B
Metabolites changed by antipurinergic
therapy in the Fragile X Model:

| Metabolite | VIP Score |
|---|---|
| Xanthine | 8.283 |
| Hypoxanthine | 6.9083 |
| Inosine | 6.3985 |
| LTB4 | 4.7929 |
| Guanosine | 4.1962 |
| 1-Methylnicotinamide | 3.4567 |
| 11-Dehydro-thromboxane B2 | 3.0285 |
| 4-hydroxyphenyllactic acid | 2.9524 |
| L-cystine | 2.8156 |
| Hexanoylcarnitine | 2.766 |
| Dihexosylceramide (18:1/24:1) | 2.7087 |
| Ceramide (d18:1/24:1) | 2.6984 |
| Ceramide (d18:1/24:0 OH) | 2.6743 |
| 2,3-Diphosphoglyceric acid | 2.6413 |
| PI (26:1) | 2.5143 |
| Dihexosylceramide (18:1/20:0) | 2.5094 |
| Ceramide (d18:1/16:0 OH) | 2.4973 |
| Trihexosylceramide 18:1/16:0 | 2.2984 |
| Cysteineglutathione disulfide | 2.2284 |
| dTDP-D-glucose | 2.1762 |
| Trihexosylceramide 18:1/22:0 | 2.1755 |
| Bismonoacylphospholipid (18:1/18:1) | 2.0984 |
| Malondialdehyde | 2.0928 |
| PC (18:0/20:3) | 2.087 |
| 3,5-Tetradecadiencarnitine | 2.0594 |
| 14,15-epoxy-5,8,11-eicosatrienoic acid | 1.9964 |
| Cardiolipin (24:1/24:1/24:1/14:1) | 1.9754 |
| Trihexosylceramide 18:1/24:1 | 1.9105 |
| 8,9-Epoxyeicosatrienoic acid | 1.8643 |
| Myristoylcarnitine | 1.8395 |
| Trihexosylceramide 18:1/24:0 | 1.8222 |
| Cholic acid | 1.8062 |
| Octanoylcarnitine | 1.7888 |
| Pimelylcarnitine | 1.7778 |
| Ceramide (d18:1/26:0) | 1.7619 |
| PG(16:0/16:0) | 1.7575 |
| Dodecenoylcarnitine | 1.7435 |
| Nicotinamide N-oxide | 1.724 |
| Dodecanoylcarnitine | 1.6983 |
| L-Homocysteic acid | 1.6739 |
| 9-Decenoylcarnitine | 1.6702 |
| Hydroxyisocaproic acid | 1.6696 |
| Propionic acid | 1.6633 |
| 5-alpha-Cholestanol | 1.6542 |
| Glyceric acid 1,3-biphosphate | 1.6112 |
| Bismonoacylphospholipid (18:1/18:0) | 1.6108 |
| 3-methylphenylacetic acid | 1.6055 |
| Cytidine | 1.5738 |
| Oxaloacetic acid | 1.5682 |
| 9-Hexadecenoylcarnitine | 1.5637 |
| Dehydroisoandrosterone 3-sulfate | 1.5627 |
| Ceramide (d18:1/20:1) | 1.5607 |
| 11(R)-HETE | 1.5384 |
| PE (38:5) | 1.5338 |
| Pyridoxamine | 1.5335 |
| 11,12-DiHETrE | 1.5284 |
| Sedoheptulose 7-phosphate | 1.5159 |
| AICAR | 1.5150 |

A simplified map of metabolism is illustrated in the form of 26 major biochemical pathways in FIG. 13. This figure shows the effect of suramin treatment on each metabolite as measured in the plasma. The magnitude of the pharmacometabolomic effect is quantified as the z-score for nearly 500 metabolites. Red indicates an increase. Green indicates a decrease. A quick visual inspection of this figure leads to several conclusions. First, 1-carbon folate and Krebs cycle metabolism are dominated by red shading, indicating a general increase in methylation pathways, and mitochondrial oxidative phosphorylation. Next, there was a generalized increase in intermediates of the SAM/SAH and glutathione metabolism. Purine metabolism showed a mixture of upregulated precursors of adenine nucleotides and downregulated inosine and guanosine precursors. There was a generalized increase in gangliosides, phospholipids, and cholesterol metabolites needed for myelin and cell membrane synthesis. Finally, there was a generalized decrease in 9 of 9 acyl-carnitine species. Acyl-carnitines accumulate when fatty acid oxidation is impaired, and decline when normal mitochondrial fatty acid oxidation is restored. Each of these pathways is a known feature of the cell danger response (CDR) (Naviaux, 2013).

Metabolic Pathway Visualization in Cytoscape.

A rendering of mammalian intermediary metabolism was constructed in Cytoscape v 3.1.1 (see, e.g., [http://]www.cytoscape.org/). Pathways represented in the network for Fragile X syndrome included the 20 metabolic pathways and the 58 metabolites that were altered by antipurinergic therapy with suramin (VIP scores >1.5). Nodes in the Cytoscape network represent metabolites within the pathways and have been colored according to the Z-score. The Z-score was computed as the arithmetic difference between the mean concentration of each metabolite in the KO-Sur treatment group and the KO-Sal control group, divided by the standard deviation in the controls. Node colors were arranged on a red-green color scale with green representing −2.00 Z-score, red representing +2.00 Z-score, and with a zero (0) Z-score represented as white. The sum of the VIP scores of those metabolites with VIP scores >1.5 for each metabolic pathway is displayed next to the pathway name.

The 20 pathways found to be altered in the Fragile X model (Table 6) were compared to the 18 metabolic pathways that were altered in the maternal immune activation (MIA) model (Example 2 below). A Venn diagram of this comparison revealed 11 pathways that were shared between these two models (FIG. 12). These were purines, the microbiome, phospholipid, sphingolipid, cholesterol, bile acids, glycolysis, the Krebs cycle, NAD+, pyrimidines, and adenosylmethionine (SAM), adenosyl-homocysteine (SAH), and glutathione (GSH) metabolism.

Data Analysis.

Group means and standard error of the means (SEM) are reported. Behavioral data were analyzed by two-way ANOVA and one-way ANOVAs (GraphPad Prism 5.0d, GraphPad Software Inc., La Jolla, Calif., USA, or Stata/SE v12.1, StataCorp, College Station, Tex., USA). Pair-wise post hoc testing was performed by the method of Tukey or Newman-Keuls. Significance was set at p<0.05. Metabolomic data were log-transformed and analyzed by multivariate partial least squares discriminant analysis (PLSDA) in MetaboAnalyst (Xia et al., 2012). Metabolites with variable importance in projection (VIP) scores greater than 1.5 were considered significant.

### Example 3

### MIA Model

Animals and Husbandry.

All studies were conducted in facilities accredited by the Association for Assessment and Accreditation of Laboratory Animal Care International (AAALAC), and followed the National Institutes of Health Guidelines for the use of animals in research. Six- to eight-week-old C57BL/6J (strain no. 000664) mice were obtained from Jackson Laboratories (Bar Harbor, Me., USA), given food and water ad libitum, identified by ear tags, and used to produce the timed matings. Animals were housed in a temperature-(22-24° C.) and humidity (40-55%)-controlled vivarium with a 12-h light-dark cycle (lights on at 0700 hours). Nulliparous dams were mated at 9-10 weeks of age. The sires were also 9-10 weeks of age. The human biological age equivalent for the C57BL/6J strain of laboratory mouse (Mus musculus) can be estimated from the following equation: 12 years for the first month, 6 years for the second month, 3 years for months 3-6 and 2.5 years for each month thereafter. Therefore, a 6-month-old mouse would be the biological equivalent of 30 years old (=12+6+3×4) on a human timeline.

Poly(IC) Preparation and Gestational Exposure.

To initiate the MIA model, pregnant dams were given two intraperitoneal injections of Poly(I:C) (Potassium salt; Sigma-Aldrich, St. Louis, Mo., USA, Cat no. P9582; >99% pure; <1% mononucleotide content). These were quantified by UV spectrophotometry. One unit (U) of poly(IC) was defined as 1 absorbance unit at 260 nm. Typically, 1U=12 μg of RNA. 0.25U/g [3 mg kg⁻¹] of poly(IC) was given on E12.5 and 0.125U g⁻¹ (1.5 mg kg⁻¹) on E17.5 as previously described. Contemporaneous control pregnancies were produced by timed matings and randomized assignment of pregnant dams to saline injection (5 μl g⁻¹ intraperitoneally (i.p.)) on E12.5 and E17.5.

Postnatal Handling and Antipurinergic Therapy (APT).

Offspring of timed matings were weaned at 3-4 weeks of age into cages of two to four animals. No mice were housed in isolation. Only males were evaluated in these studies. Littermates were identified by ear tags and distributed into different cages in order to minimize litter and dam effects. To avoid chance differences in groups selected for single-dose treatment, the saline and poly(IC) exposure groups were each balanced according to their social approach scores at 2.25 months. At 5.25 or 6.5 months of age, half the animals received a single injection of either saline (5 μl g⁻¹ i.p.) or suramin (hexasodium salt, 20 mg kg⁻¹ i.p.; Tocris Bioscience, Bristol, UK, Cat no. 1472). Beginning 2 days later, behaviors were evaluated. After completing the behavioral measurements, half of the subjects were killed after a 5-week-washout period for measurement of suramin tissue levels. For acute suramin levels, the other half was injected at 7.75 months of age and killed 2 days later for tissue level determinations.

Behavioral Testing.

Behavioral testing began at 2.25 months (9 weeks) of age. Mice were tested in social approach, rotarod, t-maze test of spontaneous alternation and light-dark box test. If abnormalities were found, treatment with suramin or saline was given at 5.25 months (21 weeks) or 6.5-6.75 months (26-27 weeks) and the testing was repeated. Only male animals were tested.

Social Approach.

Social behavior was tested as social preference (N=19-25, 2.25-month-old males per group before adult treatment with suramin. N=8-13, 6.5-month-old males per group).

Social behavior in mice can be quantified as the time spent interacting with a novel (‘stranger’) mouse compared with the total time spent interacting with either a mouse or a novel inanimate object. MIA animals showed social deficits from an early age. Single-dose APT with suramin completely reversed the social abnormalities in 6.5-month-old adults. Five weeks (5 half-lives) after suramin washout, a small residual benefit to social behavior was still detectable. The residual social benefit of APT even after 5 weeks following suramin was correlated with retained metabolomic benefits.

T-Maze.

Novelty preference was tested as spontaneous alternation behavior in the T-maze. N=19-25, 4-month-old males per group before adult treatment with suramin. (N=8-13, 5.25-month-old males per group).

Novelty preference is an innate feature of normal rodent and human behavior and a predictor of socialization and communication growth in children with ASD. The loss or suppression of novelty preference in children with ASD is associated with the phenomenon known as insistence on sameness. Preference for novelty was estimated as spontaneous alternation behavior in the T-maze. The T-maze can also be used to estimate spatial working memory, especially when food-motivated. MIA animals showed deficient novelty preference as reflected by chance (near 50%) spontaneous alternation behavior. These deficits were normalized after a single dose of suramin. Five weeks after suramin washout, no residual benefit remained.

Rotarod.

Sensorimotor coordination was tested as latency to fall on the rotarod; N=19-25, 2.5-month-old males per group before adult treatment with suramin. (N=8-13, 6.75-month-old males per group).

Previous studies have shown age-dependent, postnatal loss of cerebellar Purkinje cells in the MIA model. This can reach up to 60% of Purkinje cells lost by 4 months (16 weeks) of age. Motor coordination measured by rotarod performance is deficient in the MIA model and is critically dependent on the integrity of Purkinje cell circuits in the cerebellum. Since Purkinje cells are known to be lost in MIA animals by 4 months (16 weeks) of age, it was hypothesized that APT given later in life would have no effect. The results confirmed this. A single injection of suramin given to 6-month-old adults failed to restore normal motor coordination. Although cerebellar Purkinje cell density was not quantified in this study, our results are consistent with the notion that once Purkinje cells are lost, their function cannot be restored by APT in adult animals.

Light-Dark Box.

Certain anxiety-related and light-avoidance behaviors were tested in the light-dark box paradigm. (N=19-25, 3.5-month-old males per group).

Absence of Abnormal Behaviors Produced by Suramin.

This was assessed in the non-MIA control animals (indicated as the ‘Saline’ group) that were injected with suramin as adults (indicated as the ‘Sal-Sur’ groups in the single-dose treatment) using each of the above behavioral paradigms.

Suramin Quantitation.

Tissue samples (brainstem, cerebrum and cerebellum) were ground into powder under liquid nitrogen in a pre-cooled mortar. Powdered tissue (15-50 mg) was weighed and mixed with the internal standard trypan blue to a final concentration of 5 μM (pmol mg⁻¹) and incubated at room temperature for 10 min to permit metabolite interaction with binding proteins. Nine volumes of methanol:acetonitrile:H₂O (43:43:16) pre-chilled to −20° C. was added to produce a final solvent ratio of 40:40:20, and the samples were deproteinated and macromolecules removed by precipitation on crushed ice for 30 min. The mixture was centrifuged at 16 000 g for 10 min at 4° C. and the supernatant was transferred to a new tube and kept at −80° C. for further LC-MS/MS (liquid chromatography-tandem mass spectrometry) analysis. For plasma, 90 μl was used, to which 10 μl of 50 μM stock of trypan blue was added to achieve an internal standard concentration of 5 μM. This was incubated at room temperature for 10 min to permit metabolite interaction with binding proteins, then extracted with 4 volumes (400 μl) of pre-chilled methanol:acetonitrile (50:50) to produce a final concentration of 40:40:20 (methanol:acetonitrile:H₂O) and precipitated on ice for 10 min. Other steps were the same as for solid tissue extraction.

Suramin was analyzed on an AB SCIEX QTRAP 5500 triple quadrupole mass spectrometer equipped with a Turbo V electrospray ionization source, Shimadzu LC-20A UHPLC system, and a PAL CTC autosampler (AB SCIEX, Framingham, Mass., USA). Ten microliters of extract were injected onto a Kinetix pentafluorophenyl column (150×2.1 mm, 2.6 μm; Phenomenex, Torrance, Calif., USA) held at 30° C. for chromatographic separation. The mobile phase A was water with 20 mM ammonium acetate (NH₄OAC; pH 7) and mobile phase B was methanol with 20 mM NH₄OAC (pH 7). Elution was performed using the following gradient: 0 min-0% B, 15 min-100% B, 18 min-100% B, 18.1 min-0% B, 23 min-end. The flow rate was 300 μl min⁻¹. All the samples were kept at 4° C. during analysis. Suramin and trypan blue were detected using scheduled multiple reaction monitoring (MRM) with a dwell time of 30 ms in negative mode and retention time window of 7.5-8.5 min for suramin and 8.4-9.4 min for trypan blue. MRM transitions for the doubly charged form of suramin were 647.0 mz⁻¹ (Q1) precursor and 382.0 mz⁻¹ (Q3) product. MRM transitions for trypan blue were 435.2 (Q1) and 185.0 (Q3). Absolute concentrations of suramin were determined for each tissue using a tissue-specific standard curve to account for matrix effects, and the peak area ratio of suramin to the internal standard trypan blue. The declustering potential, collision energy, entrance potential and collision exit potential were −104, −9.5, −32 and −16.9, and −144.58, −7, −57.8 and −20.94 for suramin and trypan blue, respectively. The electrospray ionization source parameters were set as follows: source temperature 500° C.; curtain gas 30; ion source gas 1, 35; ion source gas 2 35; spray voltage −4500V. Analyst 1.6.1 was used for data acquisition and analysis. N=4-6 per tissue. Results are reported as means±s.e.m. in absolute μM (pmol μl⁻¹) concentration for plasma, and pmol mg⁻¹ wet weight for tissues.

Suramin is known not to pass the blood-brain barrier; however, no studies have looked at suramin concentrations in areas of the brain similar to the area postrema in the brainstem that lack a blood-brain barrier. After completing the behavioral studies described above, mass spectrometry was used to measure drug levels in plasma, cerebrum, cerebellum and brainstem following a 5-week period of drug washout. The plasma half-life of suramin after a single dose in mice is 1 week. No suramin was detected in any tissue after 5 weeks of drug washout. An acute injection of suramin (20 mg kg⁻¹ i.p.) to the remaining subjects was performed. After 2 days, plasma suramin was 7.64 μM±0.50, and brainstem suramin was 5.15 pmol mg⁻¹±0.49. No drug was detectable in the cerebrum or cerebellum (<0.10 pmol mg⁻¹ wet weight) in either control (Sal-Sur) or MIA (PIC-Sur) animals, consistent with an intact blood-brain barrier that excluded suramin from these tissues. In contrast to the cerebrum and cerebellum, the brainstem showed significant suramin uptake. These results are consistent with the notion that nuclei in brainstem, or their projection targets in distant sites of the brain, may mediate the dramatic behavioral effects of acute and chronic APT in this model.

Metabolomics.

Broad-spectrum analysis of 478 targeted metabolites from 44 biochemical pathways in the plasma was performed. Only male animals that had been behaviorally evaluated were tested. Samples were analyzed on an AB SCIEX QTRAP 5500 triple quadrupole mass spectrometer equipped with a Turbo V electrospray ionization source, Shimadzu LC-20A UHPLC system and a PAL CTC autosampler (AB SCIEX). Whole blood was collected 2 days after a single dose of suramin (20 mg kg⁻¹ i.p.) or saline (5 μl g⁻¹ i.p.) from animals that were lightly anesthetized with isoflurane (Med-Vet International, Mettawa, Ill., USA, Cat no. RXISO-250) in a drop jar into BD Microtainer tubes containing lithium heparin (Becton Dickinson, San Diego, Calif., USA, Ref no. 365971) by submandibular vein lancet. Plasma was separated by centrifugation at 600 g×5 min at 20° C. within 1 h of collection. Fresh lithium-heparin plasma was transferred to labeled tubes for storage at −80° C. for analysis. Typically, 45 μl of plasma was thawed on ice and transferred to a 1.7-ml Eppendorf tube. Two and one-half (2.5) microliters of a cocktail containing 35 commercial stable isotope internal standards and 2.5 μl of 310 stable isotope internal standards that were custom-synthesized in Escherichia coli and Saccharomyces cerevisiae by metabolic labeling with ¹³C-glucose and ¹³C-bicarbonate were added, mixed and incubated for 10 min at 20° C. to permit small molecules and vitamins in the internal standards to associate with plasma-binding proteins. Macromolecules (protein, DNA, RNA and so on) were precipitated by extraction with 4 volumes (200 μl) of cold (−20° C.), acetonitrile:methanol (50:50) (LCMS grade, Cat no. LC015-2.5 and GC230-4, Burdick & Jackson, Honeywell, Muskegon, Mich., USA), vortexed vigorously and incubated on crushed ice for 10 min, and then removed with centrifugation at 16000 g×10 min at 4° C. The supernatants containing the extracted metabolites and internal standards in the resulting 40:40:20 solvent mix of acetonitrile:methanol:water were transferred to labeled cryotubes and stored at −80° C. for LC-MS/MS (liquid chromatography-tandem mass spectrometry) analysis.

LC-MS/MS analysis was performed by MRM under the Analyst v1.6.1 software control in both negative and positive modes with rapid polarity switching (50 ms). Nitrogen was used for curtain gas (set to 30), collision gas (set to high) and ion source gases 1 and 2 (set to 35). The source temperature was 500° C. Spray voltage was set to −4500V in negative mode and to 5500V in positive mode. The values for Q1 and Q3 mass-to-charge ratios (mz−1), declustering potential, entrance potential, collision energy and collision cell exit potential were determined and optimized for each MRM for each metabolite. Ten microliters of extract were injected with PAL CTC autosampler into a 250 mm×2.1 mm, 5-μm Luna NH2 aminopropyl HPLC column (Phenomenex) held at 25° C. for chromatographic separation. The mobile phase was solvent A: 95% water with 23.18 mM NH₄OH (Sigma, Fluka Cat no. 17837-100ML), 20 mM formic acid (Sigma, Fluka Cat no. 09676-100ML) and 5% acetonitrile (pH 9.44); solvent B: 100% acetonitrile. Separation was achieved using the following gradient: 0 min-95% B, 4 min-B, 19 min-2% B, 22 min-2% B, 23 min-95% B, 28 min-end. The flow rate was 300 μl min⁻¹. All the samples were kept at 4° C. during analysis. The chromatographic peaks were identified using MultiQuant v2.1.1 (AB SCIEX), confirmed by manual inspection and the peak areas were integrated. The median of the peak area of stable isotope internal standards was calculated and used for the normalization of metabolite concentration across the samples and batches. N=6, 6.5-month-old males per group. Metabolite data were log-transformed before multivariate and univariate analyses.

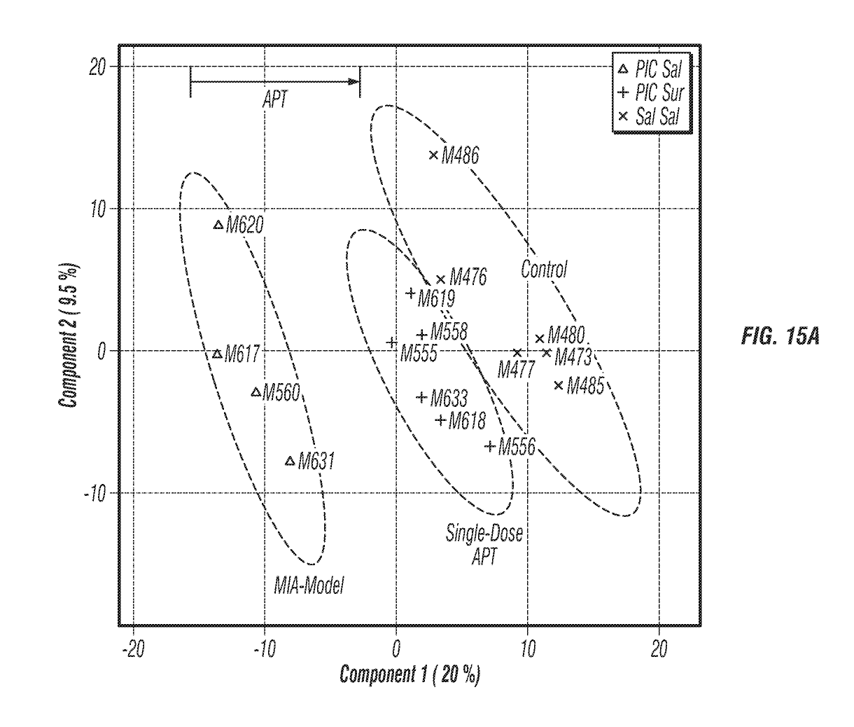

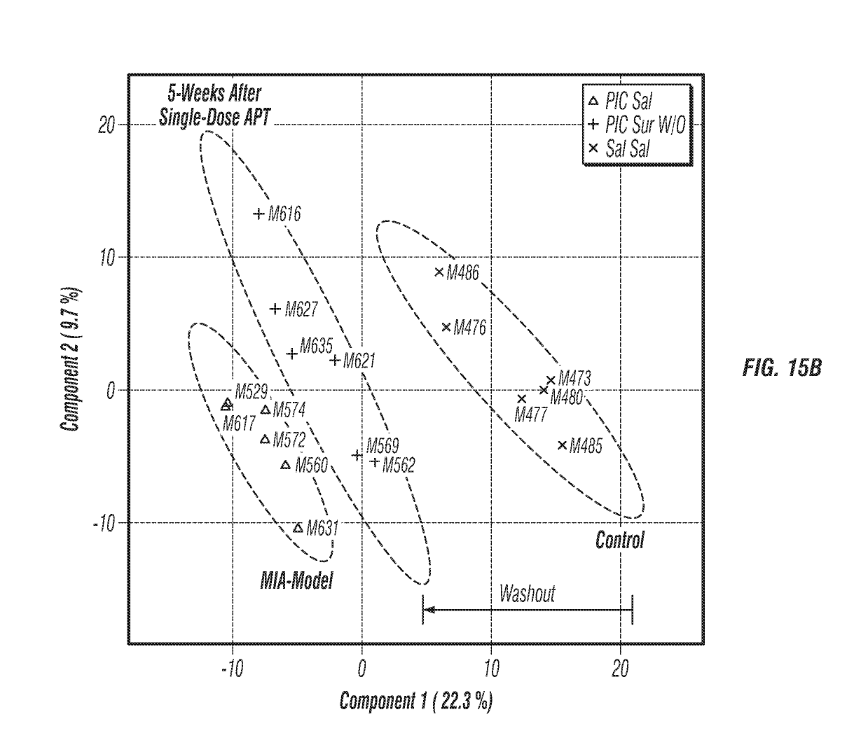

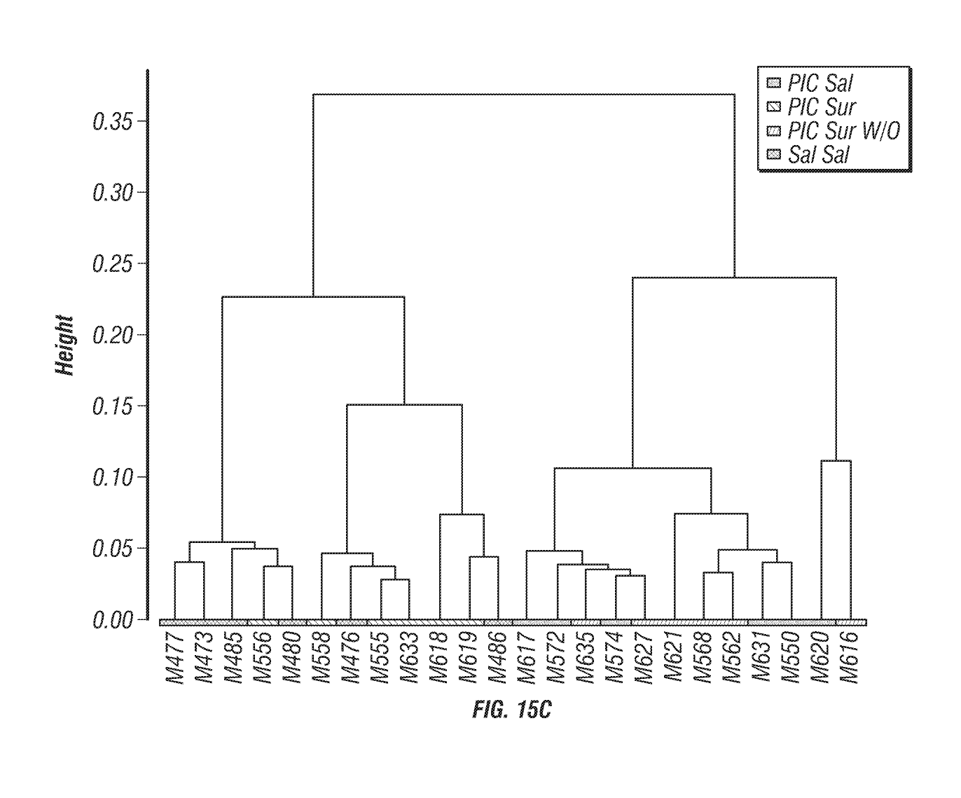

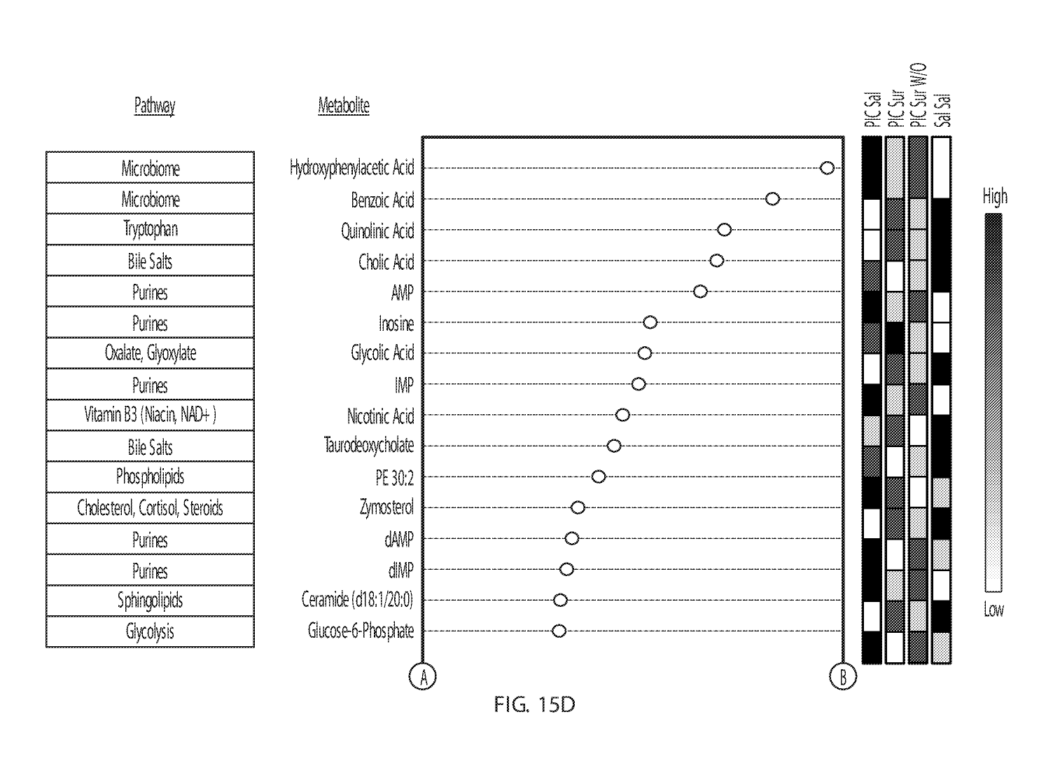

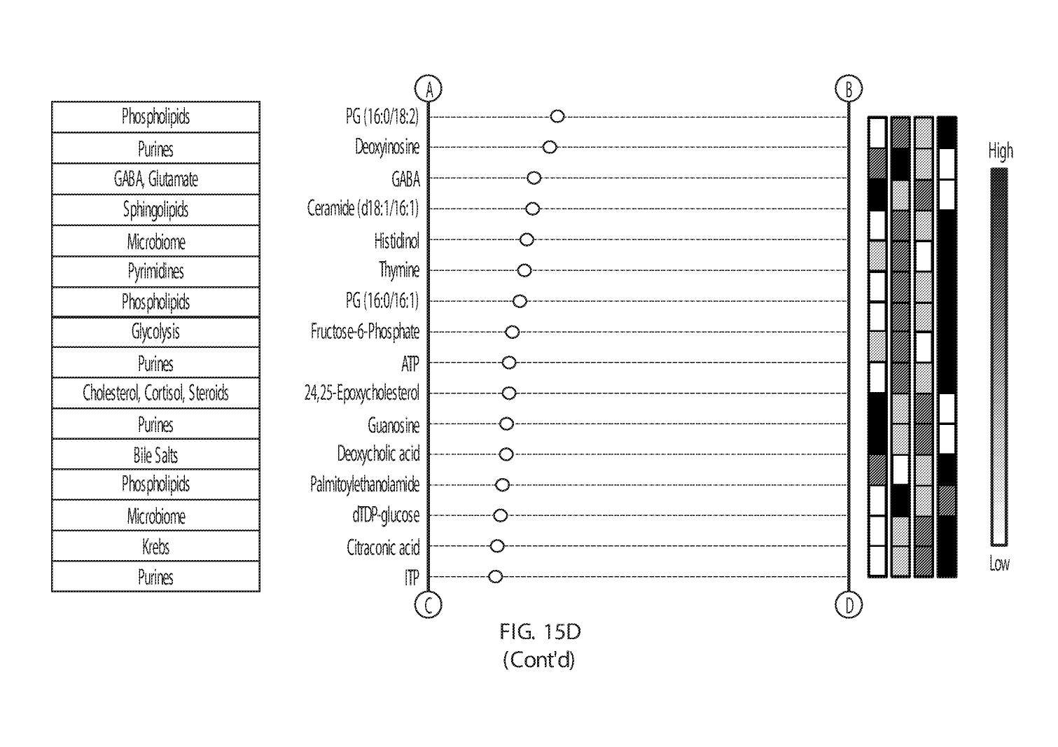

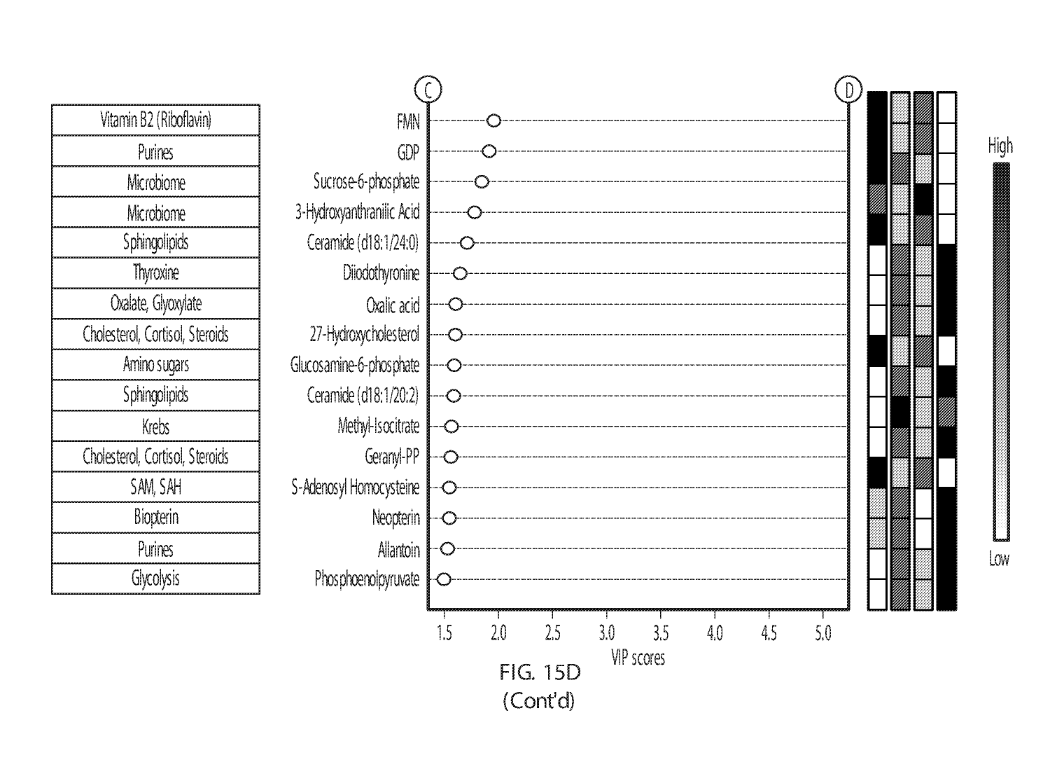

The acute metabolomic effects in plasma 2 days after single-dose treatment with suramin or saline in the same animals studied behaviorally were also analyzed. 478 metabolites were measured from 44 pathways using mass spectrometry, analyzed the data by partial least squares discriminant analysis and visualized the results by projection in two dimensions (FIGS. 15A-B). This revealed sharp differences between control and MIA animals that were substantially normalized by a single treatment with suramin (FIG. 15A). FIG. 15B shows a similar analysis that illustrates the gradual return to disease-associated metabolism after 5 weeks of drug washout. Using hierarchical cluster analysis the data show that the metabolic profiles of controls (Sal-Sal) and MIA animals that were treated with one dose of suramin (PIC-Sur) were more similar (major branch on the left of FIG. 15C) than the metabolic profiles of saline-treated MIA animals (PIC-Sal) and the MIA animals tested 5 weeks after suramin washout (PIC-Sur W/O; major branch on the right of FIG. 15C). The reason that the metabolic profile had not returned completely to pretreatment conditions (to the position of the red triangles in FIG. 15B) even after 5 weeks following a dose of suramin was not investigated but could be due to the development of metabolic memory and/or somatic epigenetic DNA changes that lasted longer than the physical presence of the drug.

FIG. 15D shows the top 48 significant metabolites found in the untreated MIA animals, ranked according to their impact by variable importance in projection (VIP) score. The columns on the right of the figure indicate the direction of the change. In 43 of the 48 (90%) discriminating metabolites, suramin treatment (PIC-Sur) resulted in a metabolic shift in concentration that was either intermediate or in the direction of and beyond that found in control animals (Sal-Sal). The biochemical pathways represented by each metabolite are indicated on the left of FIG. 15D.

The most influenced biochemical pathway in the MIA mouse was purine metabolism (Table 7). Eleven (23%) of the 48 discriminant metabolites were purines. Nine (82%) of the 11 purine metabolites were increased in the untreated MIA mice, consistent with hyperpurinergia. Only ATP and allantoin, the end product of purine metabolism in mice, were decreased in the plasma. A limitation of plasma metabolomics is that it cannot measure the effective concentration of nucleotides in the pericellular halo that defines the unstirred water layer near the cell surface where receptors and ligands meet. The concentration of ATP in the unstirred water layer is regulated according to conditions of cell health and danger in the range of 1-10 μM, which is near the EC₅₀ of most purinergic receptors. This is up to 1000-fold more concentrated than the 10-20 nM levels of ATP in compartments removed from the cell surface such as the plasma. In the plasma the data showed that suramin restored 9 (82%) of the 11 purine metabolites to more normal levels, including ATP and allantoin (FIG. 15D, right PIC-Sur column) and increased inosine and deoxyinosine to above normal.

TABLE 7
Biochemical pathways with metabolites altered in the MIA mouse model of neurodevelopmental disorders

| | | Measured | | Expected | Observed | | | Fraction | Pathway |
|---|---|---|---|---|---|---|---|---|---|
| | | metabolites | Expected | hits in a | hits in | | | of VIP | normalized |
| | | in the | pathway | sample | the top | Fold- | | explained | by single-dose |
| | | pathway | proportion | of 48 | 48 | enrichment | Impact | (% of | suramin |
| No. | Pathway | (N) | (P = N/478) | (P * 48) | metabolites | (Obs/Exp) | (Σvip) | 116.16) | treatment |
| 1 | Purine metabolism | 48 | 0.1004 | 4.8201 | 11 | 2.3 | 28.19 | 24.3% | Yes (9/11) |
| 2 | Microbiome metabolism | 32 | 0.0669 | 3.2134 | 6 | 1.9 | 17.53 | 15.1% | Yes (6/6) |
| 3 | Phospholipid metabolism | 88 | 0.1841 | 8.8368 | 4 | 0.5 | 9.76 | 8.4% | Yes (4/4) |
| 4 | Bile salt metabolism | 4 | 0.0084 | 0.4017 | 3 | 7.5 | 9.23 | 7.9% | No (0/3) |
| 5 | Sphingolipid metabolism | 72 | 0.1506 | 7.2301 | 4 | 0.6 | 8.28 | 7.1% | Yes (4/4) |
| 6 | Cholesterol, cortisol, | 19 | 0.0397 | 1.9079 | 4 | 2.1 | 8.08 | 7.0% | Yes (4/4) |
| | steroid metabolism | | | | | | | | |
| 7 | Glycolysis and | 17 | 0.0356 | 1.7071 | 3 | 1.8 | 6.25 | 5.4% | Yes (3/3) |
| | gluconeogenesis | | | | | | | | |
| 8 | Oxalate, glyxoylate | 3 | 0.0063 | 0.3013 | 2 | 6.6 | 5.02 | 4.3% | Yes (2/2) |
| | metabolism | | | | | | | | |
| 9 | Tryptophan metabolism | 11 | 0.0230 | 1.1046 | 1 | 0.9 | 4.11 | 3.5% | Yes (1/1) |
| 10 | Krebs cycle | 18 | 0.0377 | 1.8075 | 2 | 1.1 | 3.58 | 3.1% | Yes (2/2) |
| 11 | Vitamin B3 (niacin/ | 7 | 0.0146 | 0.7029 | 1 | 1.4 | 3.19 | 2.7% | Yes (1/1) |
| | NAD) metabolism | | | | | | | | |
| 12 | GABA, glutamate, | 6 | 0.0126 | 0.6025 | 1 | 1.7 | 2.33 | 2.0% | Yes (1/1) |
| | arginine, omithine, | | | | | | | | |
| | proline metabolism | | | | | | | | |
| 13 | Pyrimidine metabolism | 35 | 0.0732 | 3.5146 | 1 | 0.3 | 2.24 | 1.9% | Yes (1/1) |
| 14 | Vitamin B2 (riboflavin) | 4 | 0.0084 | 0.4017 | 1 | 2.5 | 1.97 | 1.7% | Yes (1/1) |
| | metabolism | | | | | | | | |
| 15 | Thyroxine metabolism | 1 | 0.0021 | 0.1004 | 1 | 10.0 | 1.66 | 1.4% | Yes (1/1) |
| 16 | Amino-sugar and | 10 | 0.0209 | 1.0042 | 1 | 1.0 | 1.61 | 1.4% | Yes (1/1) |
| | galactose metabolism | | | | | | | | |
| 17 | SAM, SAH, methionine, | 22 | 0.0460 | 2.2092 | 1 | 0.5 | 1.57 | 1.3% | Yes (1/1) |
| | cysteine, glutathione | | | | | | | | |
| | metabolism | | | | | | | | |
| 18 | Biopterin, neopterin, | 1 | 0.0021 | 0.1004 | 1 | 10.0 | 1.56 | 1.3% | Yes (1/1) |
| | molybdopterin | | | | | | | | |
| | metabolism | | | | | | | | |
| | | 398 | 0.8326 | 40 | 48 | | 116.16 | 100% | 94% (17/18) |
| | | (0.8326 × 478) | | (0.8326 × 48) | | | | | |

Abbreviation:
VIP, variable importance in projection.
Pathways were ranked by their impact measured by summed VIP (ΣVIP) scores. A total of 48 metabolites were found to discriminate treatment, control, washout and MIA groups by multivariate partial least squares discriminant analysis (PLSDA). Significant metabolites had VIP scores of ≧1.5. Eighteen (41%) of the 44 pathways interrogated had at least one metabolite with VIP scores ≧1.5. The total impact of these 48 metabolites corresponded to a summed VIP score of 116.16. The fractional impact of each pathway is quantified as the percent of the summed VIP score and displayed in the final column on the right in the table. Single dose APT with suramin not only corrected purine metabolism but also normalized 17 (94%) of 18 metabolic pathway abnormalitles that defined the MIA model of neurodevelopmental disorders.

Additional pathway analysis revealed a pattern of disturbances that was remarkably similar to metabolic disturbances that have been found in children with ASDs (Table 7). Eighteen of the 44 pathways were disturbed in the MIA model. The 44 pathways interrogated by this analysis are reported in Table 8. After purine metabolism, the next most influenced pathway was the microbiome. Microbiome metabolites are molecules that are produced by biochemical pathways that are absent in mammalian cells but are present in bacteria that reside in the gut microbiome. Together, purine and microbiome metabolism accounted for nearly 40% (ΣVIP=39.4%) of the impact measured by VIP scores. The two top discriminant metabolites were products of the microbiome (FIG. 15D). A total of seven pathways each contributed 5% or more to the VIP pathway impact scores (Table 7). These top seven pathways were purines, microbiome metabolism, phospholipids, bile salt metabolism, sphingolipids, cholesterol, cortisol, and steroid metabolism and glycolysis. Seventy-five percent (75%) of the metabolite VIP score impact was accounted for by metabolites in these seven pathways (Table 7). Forty-six (46) metabolites satisfied a false discovery rate threshold of less than 10% in this analysis. These were rank ordered by P-values. This univariate analysis identified 16 (35% of 46) metabolites (Table 9) that were also found by multivariate analysis across the four groups, and 30 (65%) additional metabolites that were discriminating only in pairwise group comparisons.

TABLE 8
Biochemical Pathways Interrogated

| Pathway | Metabolites |
|---|---|
| 1-Carbon, Folate, Formate, Glycine | 6 |
| Amino acid metabolism not otherwise covered | 6 |
| Amino-Sugar and Galactose Metabolism | 10 |
| Bile Salt Metabolism | 4 |
| Bioamines and Neurotransmitter Metabolism | 3 |
| Biopterin, Neopterin, Molybdopterin Metabolism | 1 |
| Biotin (Vitamin B7) Metabolism | 1 |
| Branch Chain Amino Acid Metabolism | 7 |
| Cholesterol, Cortisol, Steroid Metabolism | 19 |
| Endocannabinoid Metabolism | 1 |
| Fatty Acid Oxidation and Synthesis | 7 |
| Food Sources, Additives, Preservatives, Colorings, | 2 |
| and Dyes | |
| GABA, Glutamate, Arginine, Ornithine, Proline | 6 |
| Metabolism | |
| Glycolysis and Gluconeogenesis | 17 |
| Histidine, Histamine Metabolism | 2 |
| Isoleucine, Valine, Threonine, or Methionine | 3 |
| Metabolism | |
| Ketone Body Metabolism | 2 |
| Krebs Cycle | 18 |
| Lysine Metabolism | 2 |
| Microbiome Metabolism | 32 |
| Nitric Oxide, Superoxide, Peroxide Metabolism | 1 |
| OTC and Prescription Pharmaceutical Metabolism | 2 |
| Subtotal | 152 |
| TOTAL Pathways and Chemical Sources | 44 |
| Oxalate, Glyxoylate Metabolism | 3 |
| Pentose Phosphate, Gluconate Metabolism | 11 |
| Phosphate and Pyrophosphate Metabolism | 1 |
| Phospholipid Metabolism | 88 |
| Phytanic, Branch, Odd Chain Fatty Acids | 1 |
| Polyamine Metabolism | 4 |
| Purine Metabolism | 48 |
| Pyrimidine Metabolism | 35 |
| SAM, SAH, Methionine, Cysteine, Glutathione | 22 |
| Metabolism | |
| Sphingolipid Metabolism | 72 |
| Taurine, Hypotaurine Metabolism | 2 |
| Thryoxine Metabolism | 1 |
| Tryptophan, Kynurenine, Serotonin, Melatonin | 6 |
| Metabolism | |
| Tyrosine and Phenylalanine Metabolism | 2 |
| Urea Cycle | 5 |
| Vitamin B1 (Thiamine) Metabolism | 4 |
| Vitamin B12 (Cobalamin) Metabolism | 1 |
| Vitamin B2 (Riboflavin) Metabolism | 4 |
| Vitamin B3 (Niacin/NAD) Metabolism | 7 |
| Vitamin B5 (Pantothenate) Metabolism | 1 |
| Vitamin B6 (Pyridoxine) Metabolism | 6 |
| Vitamin C (Ascorbate) Metabolism | 2 |
| Subtotal | 326 |
| TOTAL Metabolites | 478 |

TABLE 9
Rank Order Metabolites by Univariate Analysis

| No. | Pathway | Metabolite | p-value | −Log10(p) | FDR | Fisher's LSD |
|---|---|---|---|---|---|---|
| 1 | Phospholipid Metabolism | Glycerophosphocholine | 2.47E−07 | 6.6078 | 7.70E−05 | PIC Sur-PIC Sal; Sal Sal-PIC Sal; PIC Sur-PIC Sur W/O; Sal Sal-PIC Sur W/O |
| 2 | Cholesterol, Cortisol, Steroid Metabolism | 24,25-Epoxycholesterol | 3.22E−07 | 6.4917 | 7.70E−05 | PIC Sal-PIC Sur; PIC Sal-Sal Sal; PIC Sur W/O-PIC Sur; PIC Sur W/O-Sal Sal |
| 3 | Purine Metabolism | dAMP | 5.11E−07 | 6.2918 | 7.88E−05 | PIC Sal-PIC Sur; PIC Sal-Sal Sal; PIC Sur W/O-PIC Sur; PIC Sur W/O-Sal Sal |
| 4 | Microbiome Metabolism | Hydroxyphenylacetic acid | 6.59E−07 | 6.1608 | 7.88E−05 | PIC Sal-PIC Sur; PIC Sal-Sal Sal; PIC Sur W/O-PIC Sur; PIC Sur W/O-Sal Sal |
| 5 | Krebs Cycle | Oxaloacetic acid | 0.00018264 | 3.7384 | 0.01746 | PIC Sur-PIC Sal; Sal Sal-PIC Sal; PIC Sur-PIC Sur W/O; Sal Sal-PIC Sur W/O |
| 6 | Phospholipid Metabolism | Palmpoylethanolamide | 0.00024171 | 3.6167 | 0.018301 | PIC Sur-PIC Sal; Sal Sal-PIC Sal; PIC Sur-PIC Sur W/O; Sal Sal-PIC Sur W/O |
| 7 | Pyrimidine Metabolism | Deoxyuridine | 0.00029313 | 3.5329 | 0.018301 | PIC Sal-PIC Sur; PIC Sal-Sal Sal; PIC Sur W/O-PIC Sur; PIC Sur W/O-Sal Sal |
| 8 | Tryptophan Metabolism | Kynurenic acid | 0.00032056 | 3.4941 | 0.018301 | PIC Sur-PIC Sal; Sal Sal-PIC Sal; PIC Sur-PIC Sur W/O; PIC Sur-Sal Sal |
| 9 | Pyrimidine Metabolism | Uridine | 0.00034459 | 3.4627 | 0.018301 | PIC Sur-PIC Sal; Sal Sal-PIC Sal; PIC Sur-PIC Sur W/O; Sal Sal-PIC Sur W/O |
| 10 | Purine Metabolism | ATP | 0.00043906 | 3.3575 | 0.020987 | PIC Sur-PIC Sal; Sal Sal-PIC Sal; PIC Sur-PIC Sur W/O; Sal Sal-PIC Sur W/O |
| 11 | Purine Metabolism | Adenine | 0.00060284 | 3.2198 | 0.025208 | PIC Sur-PIC Sal; Sal Sal-PIC Sal; PIC Sur-PIC Sur W/O; Sal Sal-PIC Sur W/O |
| 12 | Microbiome Metabolism | 2,3-Dihydroxybenzoate | 0.00063285 | 3.1987 | 0.025208 | PIC Sur-PIC Sal; Sal Sal-PIC Sal; PIC Sur-PIC Sur W/O; Sal Sal-PIC Sur W/O |
| 13 | Microbiome Metabolism | 2-oxo-4-methylthiobutanoate | 0.00719514 | 3.143 | 0.025357 | PIC Sur-PIC Sal; Sal Sal-PIC Sal; PIC Sur-PIC Sur W/O; Sal Sal-PIC Sur W/O |
| 14 | Pyrimidine Metabolism | Thymine | 0.00074269 | 3.1292 | 0.025357 | PIC Sur-PIC Sal; Sal Sal-PIC Sal; PIC Sur-PIC Sur W/O; Sal Sal-PIC Sur W/O |
| 15 | Vitamin B6 (Pyridoxine) Metabolism | Nicolnate | 0.00010241 | 2.9897 | 0.032629 | Sal Sal-PIC Sal; PIC Sur-PIC Sur W/O; Sal Sal-PIC Sur W/O |
| 16 | Sphingolipid Metabolism | Ceramide 22.0 | 0.0010922 | 2.9617 | 0.032629 | PIC Sur-PIC Sal; Sal Sal-PIC Sal; PIC Sur-PIC Sur W/O; Sal Sal-PIC Sur W/O |
| 17 | Phospholipid Metabolism | PC(18:0/20:3) | 0.0014321 | 2.844 | 0.037644 | PIC Sur-PIC Sal; Sal Sal-PIC Sal; PIC Sur-PIC Sur W/O; Sal Sal-PIC Sur W/O |
| 18 | Tryptophan Metabolism | Oxinolinic Acid | 0.0014598 | 2.8357 | 0.037644 | Sal Sal-PIC Sal; Sal Sal-PIC Sur; Sal Sal-PIC Sur W/O |
| 19 | Glycolysis, Gluconeogenesis, Galactose Metabolism | D-Fructose 6-phosphate | 0.0017065 | 2.7679 | 0.037644 | PIC Sur-PIC Sal; Sal Sal-PIC Sal; PIC Sur-PIC Sur W/O; Sal Sal-PIC Sur W/O |
| 20 | Fatty Acid Oxidation and Synthesis | Oleic acid | 0.0017085 | 2.7674 | 0.037644 | PIC Sur-PIC Sal; PIC Sur-PIC Sur W/O; Sal Sal-PIC Sur W/O |
| 21 | Microbiome Metabolism | Benzole acid | 0.0017142 | 2.7659 | 0.037644 | Sal Sal-PIC Sal; Sal Sal-PIC Sur; Sal Sal-PIC Sur W/O |
| 22 | Pyrimidine Metabolism | Carbamoyl-phosphate | 0.0018628 | 2.7298 | 0.037644 | PIC Sal-PIC Sur W/O; PIC Sur-PIC Sur W/O; Sal Sal-PIC Sur W/O |
| 23 | Vitamin B5 (Pantothenate) Metabolism | Pantomeric acid | 0.0018832 | 2.7251 | 0.037644 | PIC Sal-PIC Sur; PIC Sal-Sal Sal; PIC Sur W/O-PIC Sur; PIC Sur W/O-Sal Sal |
| 24 | SAM, SAH, Methionine, Cysteine, Glutathione Metabolism | Dimethylglycine | 0.0018901 | 2.7235 | 0.037644 | PIC Sur-PIC Sal; PIC Sur-PIC Sur W/O; PIC Sur-Sal Sal |
| 25 | Phospholipid Metabolism | N-oleoylethanotamine | 0.0029363 | 2.5322 | 0.052436 | PIC Sur-PIC Sal; Sal Sal-PIC Sal; PIC Sur-PIC Sur W/O |
| 26 | Microbiome Metabolism | Xanthosine | 0.0029631 | 2.5283 | 0.052436 | PIC Sur-PIC Sal; PIC Sur-PIC Sur W/O |
| 27 | Phospholipid Metabolism | Ethanolamine | 0.003045 | 2.5164 | 0.052436 | PIC Sur-PIC Sal; PIC Sur-PIC Sur W/O; Sal Sal-PIC Sur W/O |
| 28 | Cholesterol, Cortisol, Steroid Metabolism | 24-Dihydrotanosterol | 0.0030716 | 2.5126 | 0.052436 | PIC Sur-PIC Sal; Sal Sal-PIC Sal; PIC Sur-PIC Sur W/O; Sal Sal-PIC Sur W/O |
| 29 | Vitamin B6 (Pyridoxine) Metabolism | 4-Pyridoxic acid | 0.0032599 | 2.4668 | 0.053732 | PIC Sur-PIC Sal; Sal Sal-PIC Sal; PIC Sur-PIC Sur W/O; Sal Sal-PIC Sur W/O |
| 30 | Purine Metabolism | γ-methylguanosine | 0.0035257 | 2.4528 | 0.056176 | PIC Sal-PIC Sur; PIC Sal-Sal Sal; PIC Sur W/O-PIC Sur; PIC Sur W/O-Sal Sal |
| 31 | Krebs Cycle | Succinic acid | 0.0039805 | 2.4001 | 0.059459 | PIC Sur-PIC Sal; Sal Sal-PIC Sal; PIC Sur-PIC Sur W/O; Sal Sal-PIC Sur W/O |
| 32 | Microbiome Metabolism | 3-methylphenylacetic acid | 0.0039805 | 2.4001 | 0.059459 | Sal Sal-PIC Sal; PIC Sur-PIC Sur W/O; Sal Sal-PIC Sur W/O |
| 33 | Tyrosine and Phenylalamine Metabolism | Tyrosine | 0.0043104 | 2.3655 | 0.062053 | PIC Sur-PIC Sal; Sal Sal-PIC Sal; PIC Sur-PIC Sur W/O; Sal Sal-PIC Sur W/O |
| 34 | Pentose Phosphate, Glucanate Metabolism | D-Ribose-5-phosphate | 0.0044273 | 2.3539 | 0.062053 | PIC Sur-PIC Sal; PIC Sur-PIC Sur W/O; Sal Sal-PIC Sur W/O |
| 35 | Krebs Cycle | 2-Hydroxyglutarate | 0.0045436 | 2.3426 | 0.062053 | PIC Sur-PIC Sal; Sal Sal-PIC Sal; PIC Sur-PIC Sur W/O |
| 36 | Microbiome Metabolism | 3-Hydroxyanthranlic acid | 0.0047418 | 2.3241 | 0.06296 | PIC Sal-PIC Sur; PIC Sal-Sal Sal; PIC Sur W/O-PIC Sur; PIC Sur W/O-Sal Sal |
| 37 | Branch Chain Amino Acid Metabolism | 4-methyl-2-oxopentanoic acid | 0.0050399 | 2.2976 | 0.065109 | PIC Sal-Sal Sal; PIC Sur-Sal Sal; PIC Sur W/O-Sal Sal |
| 38 | Bile salt Metabolism | Deoxycholic acid | 0.0053945 | 2.268 | 0.067857 | Sal Sal-PIC Sal; Sal Sal-PIC Sur; Sal Sal-PIC Sur W/O |
| 39 | Fatty Acid Oxidation and Synthesis | Carniitine | 0.005777 | 2.2383 | 0.070579 | PIC Sal-PIC Sur; PIC Sur W/O-PIC Sur; Sal Sal-PIC Sur |
| 40 | Thyroxine Metabolism | Diclodothyronine | 0.0059062 | 2.2287 | 0.070579 | PIC Sur-PIC Sal; Sal Sal-PIC Sal; Sal Sal-PIC Sur W/O |
| 41 | Purine Metabolism | Alantoin | 0.0065793 | 2.1818 | 0.076705 | PIC Sur-PIC Sal; Sal Sal-PIC Sal; PIC Sur-PIC Sur W/O; Sal Sal-PIC Sur W/O |
| 42 | Bile salt Metabolism | Taurodeoxycholic acid | 0.00709 | 2.1494 | 0.08069 | Sal Sal-PIC Sal; Sal Sal-PIC Sur; Sal Sal-PIC Sur W/O |
| 43 | Microbiome Metabolism | p-Hydroxybenzoate | 0.0081414 | 2.0893 | 0.090502 | PIC Sal-PIC Sur; PIC Sal-Sal Sal |
| 44 | Branch Chain Amino Acid Metabolism | Hydroxylsocaproic acid | 0.0085126 | 2.0699 | 0.091299 | PIC Sur-PIC Sal; Sal Sal-PIC Sal; Sal Sal-PIC Sur W/O |
| 45 | SAM, SAH, Methionine, Cysteine, Glutathione Metabolism | Reduced glutathione | 0.0085951 | 2.0658 | 0.091299 | Sal Sal-PIC Sal; Sal Sal-PIC Sur W/O |
| 46 | Amino Acid Metabolism not otherwise covered | Asparagine | 0.0088664 | 2.0523 | 0.092133 | Sal Sal-PIC Sal; PIC Sur-PIC Sur W/O; Sal Sal-PIC Sur W/O |

Restoration of normal purine metabolism by APT led to the concerted normalization of 17 (94%) of the 18 biochemical pathway disturbances that characterized the MIA model (Table 7; far right column). Only the bile salt pathway was not restored by suramin (Table 7, FIG. 15D). The three bile salt metabolites were highest in the plasma of control animals (FIG. 15D; Sal-Sal), lower in MIA animals (FIG. 15D; PIC-Sal) and made even lower by suramin (FIG. 15D; PIC-Sur). Overall, the data show that restoration of normal purine metabolism with APT led to the concerted improvement in both the behavioral and metabolic abnormalities in this model.

Data Analysis.

Animals were randomized into active (suramin) and mock (saline) treatment groups at ˜6 months of age. Group means and s.e.m. are reported. Behavioral data involving more than two groups were analyzed by two-way analysis of variance (ANOVA) and one-way ANOVAs (GraphPad Prism 5.0d, GraphPad Software Inc., La Jolla, Calif., USA). Pair-wise post hoc testing was performed by the method of Tukey. Repeated measures ANOVA with prenatal treatment and drug as between subject factors and stimulus (mouse/cup) on time spent with mouse or cup was used as an additional test of social preference. Student's t-test was used for comparisons involving the two groups. Significance was set at P<0.05. Bonferroni post hoc correction was used to control for multiple hypothesis testing when t-tests were used to test social preference in two or more experimental groups. Metabolomic data were analyzed using multivariate partial least squares discriminant analysis, Ward hierarchical clustering and univariate one-way ANOVA with pairwise comparisons and post hoc correction by Fisher's least significant difference test in MetaboAnalyst.

The results show that purine metabolism is a master regulatory pathway in the MIA model (Table 7, FIG. 15D, Table 8). Correction of purine metabolism with APT restored normal social behavior and novelty preference. Comprehensive metabolomic analysis revealed disturbances in several other metabolic pathways relevant to children with ASDs. These included disturbances in microbiome, phospholipid, cholesterol/sterol, sphingolipid, glycolytic and bile salt metabolism (Table 7). The top, non-microbiome-associated metabolite was quinolinic acid (FIG. 15D), which was decreased in the MIA model. Quinolinic acid is a product of the indoleamine 2,3-dioxygenase pathway of tryptophan metabolism. Interestingly, abnormalities in purine, tryptophan, microbiome, phospholipid, cholesterol/sterol and sphingolipid metabolism have each been reported in children with ASDs. Abnormalities in purine metabolism, tryptophan, cholesterol/sterol, sphingolipid and phospholipid metabolism have also been described in schizophrenia. Although the detailed metabolic features of ASD and schizophrenia are different, these disorders share biochemical pathway disturbances that reveal the persistent activation of the evolutionarily conserved CDR22 in both ASD and schizophrenia. These data show that the metabolic disturbances in the MIA model and human ASD and schizophrenia are similar and provide strong support for the biochemical validity of this animal model.

Table 10 provide a list of metabolites measured in the various embodiments described herein. In embodiments of the disclosure the full metabolite list can be probed or subsets thereof. Any combination of the metabolites can be used for diagnostics or for generating various metabolite profiles. In addition, Table 10 provides a list of the metabolites and their associated metabolic pathway. One of skill in the art can readily determine the metabolic pathway associated with the metabolite for determining a metabolomics profile.

TABLE 10

| Number | Metabolic Pathway | Chemical Name |
|---|---|---|
| 1 | 1-Carbon, Folate, Formate, Glycine Metabolism | N5-Formyl-THF |
| 2 | 1-Carbon, Folate, Formate, Glycine Metabolism | 5-Methyl-5,6,7,8- |
| | | tetrahydromethanopterin |
| 3 | 1-Carbon, Folate, Formate, Glycine Metabolism | Dihydrofolic acid_neg |
| 4 | 1-Carbon, Folate, Formate, Glycine Metabolism | Betaine |
| 5 | 1-Carbon, Folate, Formate, Glycine Metabolism | Betaine aldehyde |
| 6 | 1-Carbon, Folate, Formate, Glycine Metabolism | Folic acid_neg |
| 7 | 1-Carbon, Folate, Formate, Glycine Metabolism | Glycine |
| 8 | Amino Acid Metabolism not otherwise covered | Alanine |
| 9 | Amino Acid Metabolism not otherwise covered | L-Asparagine_pos |
| 10 | Amino Acid Metabolism not otherwise covered | D-Aspartic acid |
| 11 | Amino Acid Metabolism not otherwise covered | L-Serine |
| 12 | Amino Acid Metabolism not otherwise covered | L-Threonine_neg |
| 13 | Amino Acid Metabolism not otherwise covered | L-Threonine_pos |
| 14 | Antibiotics, Pesticides, and Xenobiotic Metabolism | Ampicillin |
| 15 | Antibiotics, Pesticides, and Xenobiotic Metabolism | Metronidazole |
| 16 | Antibiotics, Pesticides, and Xenobiotic Metabolism | Penicillin G |
| 17 | Antibiotics, Pesticides, and Xenobiotic Metabolism | Sulfanilamide |
| 18 | Antibiotics, Pesticides, and Xenobiotic Metabolism | Tetracycline |
| 19 | Antibiotics, Pesticides, and Xenobiotic Metabolism | Trypan blue |
| 20 | Antibiotics, Pesticides, and Xenobiotic Metabolism | Amoxicillin |
| 21 | Antibiotics, Pesticides, and Xenobiotic Metabolism | Amphotericin B |
| 22 | Antibiotics, Pesticides, and Xenobiotic Metabolism | Atrazine |
| 23 | Antibiotics, Pesticides, and Xenobiotic Metabolism | Atrazine-desethyl |
| 24 | Bile Salt Metabolism | Chenodeoxycholic acid |
| 25 | Bile Salt Metabolism | Chenodeoxyglycocholic acid |
| 26 | Bile Salt Metabolism | Cholic acid |
| 27 | Bile Salt Metabolism | Deoxycholic acid |
| 28 | Bile Salt Metabolism | Glycocholic acid |
| 29 | Bile Salt Metabolism | Taurochenodesoxycholic acid |
| 30 | Bile Salt Metabolism | Taurocholic acid |
| 31 | Bile Salt Metabolism | Taurodeoxycholic acid |
| 32 | Bioamines and Neurotransmitter Metabolism | Acetylcholine |
| 33 | Bioamines and Neurotransmitter Metabolism | Choline |
| 34 | Bioamines and Neurotransmitter Metabolism | Dopamine |
| 35 | Bioamines and Neurotransmitter Metabolism | D-Glutamic acid |
| 36 | Bioamines and Neurotransmitter Metabolism | L-Glutamine |
| 37 | Bioamines and Neurotransmitter Metabolism | Homovanillic acid |
| 38 | Bioamines and Neurotransmitter Metabolism | Metanephrine |
| 39 | Bioamines and Neurotransmitter Metabolism | Normetanephrine |
| 40 | Bioamines and Neurotransmitter Metabolism | Beta-Alanine |
| 41 | Bioamines and Neurotransmitter Metabolism | Epinephrine |
| 42 | Bioamines and Neurotransmitter Metabolism | Norepinephrine |
| 43 | Bioamines and Neurotransmitter Metabolism | N-Acetylaspartylglutamic acid |
| 44 | Bioamines and Neurotransmitter Metabolism | N-Acetyl-L-aspartic acid |
| 45 | Bioamines and Neurotransmitter Metabolism | Octopamine |
| 46 | Biopterin, Neopterin, Molybdopterin Metabolism | Neopterin |
| 47 | Biopterin, Neopterin, Molybdopterin Metabolism | Tetrahydrobiopterin |
| 48 | Biotin Metabolism | Biotin |
| 49 | Branch Chain Amino Acid Metabolism | 2-Hydroxy-3-methylbutyric |
| | | acid |
| 50 | Branch Chain Amino Acid Metabolism | Alpha-ketoisovaleric acid |
| 51 | Branch Chain Amino Acid Metabolism | 3-Hydroxyisovaleryl Carnitine |
| 52 | Branch Chain Amino Acid Metabolism | Alpha-ketoisovaleric acid |
| 53 | Branch Chain Amino Acid Metabolism | Ketoleucine |
| 54 | Branch Chain Amino Acid Metabolism | Hydroxyisocaproic acid |
| 55 | Branch Chain Amino Acid Metabolism | L-Isoleucine |
| 56 | Branch Chain Amino Acid Metabolism | Isovalerylcarnitine |
| 57 | Branch Chain Amino Acid Metabolism | L-Valine |
| 58 | Branch Chain Amino Acid Metabolism | 3-Hydroxyiso-/ |
| | | butyrylcarnitine |
| 59 | Branch Chain Amino Acid Metabolism | 2-Methylbutyroylcarnitine |
| 60 | Branch Chain Amino Acid Metabolism | Tiglylcarnitine |
| 61 | Branch Chain Amino Acid Metabolism | 3-Hydroxyisovaleryl-/2- |
| | | methylbutyrylcarnitine |
| 62 | Cardiolipin Metabolism | CL (14:1/14:1/14:1/15:1) |
| 63 | Cardiolipin Metabolism | CL (15:0/15:0/15:0/16:1) |
| 64 | Cardiolipin Metabolism | CL (18:2/18:1/18:1/20:4) |
| 65 | Cardiolipin Metabolism | CL (18:2/18:2/16:1/16:1) |
| 66 | Cardiolipin Metabolism | CL (18:2/18:2/18:1/18:1) |
| 67 | Cardiolipin Metabolism | CL (18:2/18:2/18:2/16:1) |
| 68 | Cardiolipin Metabolism | CL (18:2/18:2/18:2/18:1) |
| 69 | Cardiolipin Metabolism | CL (18:2/18:2/18:2/18:2) |
| 70 | Cardiolipin Metabolism | CL (18:2/18:2/18:2/20:4) |
| 71 | Cardiolipin Metabolism | CL (18:2/18:2/18:2/22:6) |
| 72 | Cardiolipin Metabolism | CL (22:1/22:1/22:1/14:1) |
| 73 | Cardiolipin Metabolism | CL (24:1/24:1/24:1/14:1) |
| 74 | Cholesterol, Cortisol, Non-Gonadal Steroid | 27-Hydroxycholesterol |
| | Metabolism | |
| 75 | Cholesterol, Cortisol, Non-Gonadal Steroid | 22R-Hydroxycholesterol |
| | Metabolism | |
| 76 | Cholesterol, Cortisol, Non-Gonadal Steroid | 24,25-Dihydrolanosterol |
| | Metabolism | |
| 77 | Cholesterol, Cortisol, Non-Gonadal Steroid | 24-Hydroxycholesterol |
| | Metabolism | |
| 78 | Cholesterol, Cortisol, Non-Gonadal Steroid | 24,25-Epoxycholesterol |
| | Metabolism | |
| 79 | Cholesterol, Cortisol, Non-Gonadal Steroid | 25-Hydroxycholesterol |
| | Metabolism | |
| 80 | Cholesterol, Cortisol, Non-Gonadal Steroid | 3-hydroxy-3-methylglutaryl- |
| | Metabolism | CoA |
| 81 | Cholesterol, Cortisol, Non-Gonadal Steroid | 4-beta-Hydroxycholesterol |
| | Metabolism | |
| 82 | Cholesterol, Cortisol, Non-Gonadal Steroid | 5,6 alpha-Epoxycholesterol |
| | Metabolism | |
| 83 | Cholesterol, Cortisol, Non-Gonadal Steroid | 5,6 beta-Epoxycholesterol |
| | Metabolism | |
| 84 | Cholesterol, Cortisol, Non-Gonadal Steroid | 7a-Hydroxycholesterol |
| | Metabolism | |
| 85 | Cholesterol, Cortisol, Non-Gonadal Steroid | 7-Dehydrocholesterol |
| | Metabolism | |
| 86 | Cholesterol, Cortisol, Non-Gonadal Steroid | 7-ketocholesterol |
| | Metabolism | |
| 87 | Cholesterol, Cortisol, Non-Gonadal Steroid | Aldosterone |
| | Metabolism | |
| 88 | Cholesterol, Cortisol, Non-Gonadal Steroid | Cholestenone |
| | Metabolism | |
| 89 | Cholesterol, Cortisol, Non-Gonadal Steroid | 5alpha-Cholestanol |
| | Metabolism | |
| 90 | Cholesterol, Cortisol, Non-Gonadal Steroid | Cholesterol |
| | Metabolism | |
| 91 | Cholesterol, Cortisol, Non-Gonadal Steroid | Cholesteryl sulfate |
| | Metabolism | |
| 92 | Cholesterol, Cortisol, Non-Gonadal Steroid | Desmosterol |
| | Metabolism | |
| 93 | Cholesterol, Cortisol, Non-Gonadal Steroid | Farnesyl diphosphate |
| | Metabolism | |
| 94 | Cholesterol, Cortisol, Non-Gonadal Steroid | Geranyl-PP |
| | Metabolism | |
| 95 | Cholesterol, Cortisol, Non-Gonadal Steroid | Lanosterin |
| | Metabolism | |
| 96 | Cholesterol, Cortisol, Non-Gonadal Steroid | Lathosterol |
| | Metabolism | |
| 97 | Cholesterol, Cortisol, Non-Gonadal Steroid | Mevalonic acid |
| | Metabolism | |
| 98 | Cholesterol, Cortisol, Non-Gonadal Steroid | Zymosterol |
| | Metabolism | |
| 99 | Cholesterol, Cortisol, Non-Gonadal Steroid | Ergosterol |
| | Metabolism | |
| 100 | Cholesterol, Cortisol, Non-Gonadal Steroid | Hydrocortisone |
| | Metabolism | |
| 101 | Cholesterol, Cortisol, Non-Gonadal Steroid | Corticosterone |
| | Metabolism | |
| 102 | Drugs of Abuse | delta9-Tetrahydrocannabinol |
| 103 | Drugs of Abuse | delta-9-THC carboxylic acid A |
| 104 | Drugs of Abuse | gamma-Hydroxybutyric acid |
| 105 | Drugs of Abuse | Dihydrocodeine |
| 106 | Drugs of Abuse | Amphetamine |
| 107 | Drugs of Abuse | Methadone |
| 108 | Drugs of Abuse | Ketamine |
| 109 | Drugs of Abuse | Heroin |
| 110 | Drugs of Abuse | Lysergide |
| 111 | Drugs of Abuse | Mescaline |
| 112 | Drugs of Abuse | Methamphetamine |
| 113 | Drugs of Abuse | THC-COOH |
| 114 | Drugs of Abuse | THC-OH |
| 115 | Drugs of Abuse | Morphine-3-beta-D- |
| | | glucuronide |
| 116 | Drugs of Abuse | Oxycodone |
| 117 | Drugs of Abuse | Psilocin |
| 118 | Drugs of Abuse | Cocaine |
| 119 | Drugs of Abuse | Codeine |
| 120 | Drugs of Abuse | Morphine |
| 121 | Drugs of Abuse | Hydrocodone |
| 122 | Drugs of Abuse | Hydromorphone |
| 123 | Drugs of Abuse | Meperidine |
| 124 | Drugs of Abuse | Oxymorphone |
| 125 | Eicosanoid and Resolvin Metabolism | Resolvin D1 |
| 126 | Eicosanoid and Resolvin Metabolism | 13S-hydroxyoctadecadienoic |
| | | acid |
| 127 | Eicosanoid Metabolism | 11-Dehydro-thromboxane B2 |
| 128 | Eicosanoid Metabolism | 11(R)-HETE |
| 129 | Eicosanoid Metabolism | 11,12-DiHETrE |
| 130 | Eicosanoid Metabolism | 11,12-Epoxyeicosatrienoic |
| | | acid |
| 131 | Eicosanoid Metabolism | 12-HETE |
| 132 | Eicosanoid Metabolism | 13,14-Dihydro-15-keto PGF2a |
| 133 | Eicosanoid Metabolism | 14,15-DHET |
| 134 | Eicosanoid Metabolism | 14,15-epoxy-5,8,11- |
| | | eicosatrienoic acid |
| 135 | Eicosanoid Metabolism | 15(S)-HETE |
| 136 | Eicosanoid Metabolism | 2,3-Dinor TXB₂ |
| 137 | Eicosanoid Metabolism | 20-Hydroxyeicosatetraenoic |
| | | acid |
| 138 | Eicosanoid Metabolism | 5-HETE |
| 139 | Eicosanoid Metabolism | 5-HPETE |
| 140 | Eicosanoid Metabolism | 5,6-DHET |
| 141 | Eicosanoid Metabolism | 6-Keto-prostaglandin F1a |
| 142 | Eicosanoid Metabolism | 8-HETE |
| 143 | Eicosanoid Metabolism | 8-Isoprostaglandin F2a |
| 144 | Eicosanoid Metabolism | 8,9-DiHETrE |
| 145 | Eicosanoid Metabolism | 8,9-Epoxyeicosatrienoic acid |
| 146 | Eicosanoid Metabolism | 9-HETE |
| 147 | Eicosanoid Metabolism | Arachidonic Acid |
| 148 | Eicosanoid Metabolism | Arachidonyl carnitine |
| 149 | Eicosanoid Metabolism | LTB4 |
| 150 | Eicosanoid Metabolism | LTC4 |
| 151 | Eicosanoid Metabolism | LTD4 |
| 152 | Eicosanoid Metabolism | LTE4 |
| 153 | Eicosanoid Metabolism | LXA4 |
| 154 | Eicosanoid Metabolism | LXB4 |
| 155 | Eicosanoid Metabolism | Prostaglandin D2 |
| 156 | Eicosanoid Metabolism | Prostaglandin E2 |
| 157 | Eicosanoid Metabolism | PGF2alpha |
| 158 | Eicosanoid Metabolism | Prostaglandin J2 |
| 159 | Eicosanoid Metabolism | Tetranor-PGEM |
| 160 | Eicosanoid Metabolism | Tetranor-PGFM |
| 161 | Eicosanoid Metabolism | Thromboxane B2 |
| 162 | Endocannabinoid Metabolism | 2-Arachidonylglycerol |
| 163 | Endocannabinoid Metabolism | Anandamide |
| 164 | Fatty Acid Oxidation and Synthesis | DL-2-Aminooctanoic acid |
| 165 | Fatty Acid Oxidation and Synthesis | 2-Ketohexanoic acid |
| 166 | Fatty Acid Oxidation and Synthesis | Carnitine |
| 167 | Fatty Acid Oxidation and Synthesis | Decanoylcarnitine |
| 168 | Fatty Acid Oxidation and Synthesis | Docosahexaenoic acid |
| 169 | Fatty Acid Oxidation and Synthesis | Dodecanoylcarnitine |
| 170 | Fatty Acid Oxidation and Synthesis | Eicosapentaenoic acid |
| 171 | Fatty Acid Oxidation and Synthesis | Glutarylcarnitine |
| 172 | Fatty Acid Oxidation and Synthesis | Hexanoylcarnitine |
| 173 | Fatty Acid Oxidation and Synthesis | L-acetylcarnitine |
| 174 | Fatty Acid Oxidation and Synthesis | Linoleic acid |
| 175 | Fatty Acid Oxidation and Synthesis | Maleic acid |
| 176 | Fatty Acid Oxidation and Synthesis | Malonyl-CoA |
| 177 | Fatty Acid Oxidation and Synthesis | Malonylcarnitine |
| 178 | Fatty Acid Oxidation and Synthesis | Myristoylcarnitine |
| 179 | Fatty Acid Oxidation and Synthesis | Octadecanoylcarnitine |
| 180 | Fatty Acid Oxidation and Synthesis | Oleic acid |
| 181 | Fatty Acid Oxidation and Synthesis | L-Palmitoylcarnitine |
| 182 | Fatty Acid Oxidation and Synthesis | Trimethylamine-N-oxide |
| 183 | Fatty Acid Oxidation and Synthesis | 9-Decenoylcarnitine |
| 184 | Fatty Acid Oxidation and Synthesis | Dodecenoylcarnitine |
| 185 | Fatty Acid Oxidation and Synthesis | 3-Hydroxydodecanoylcarnitine |
| 186 | Fatty Acid Oxidation and Synthesis | Tetradecanoylcarnitnine |
| 187 | Fatty Acid Oxidation and Synthesis | 3,5-Tetradecadiencarnitine |
| 188 | Fatty Acid Oxidation and Synthesis | 3-Hydroxy-cis-5- |
| | | tetradecenoylcarnitine |
| 189 | Fatty Acid Oxidation and Synthesis | 9-Hexadecenoylcarnitine |
| 190 | Fatty Acid Oxidation and Synthesis | 3- |
| | | Hydroxyhexadecenoylcarnitine |
| 191 | Fatty Acid Oxidation and Synthesis | Hexadecandioylcarnitine |
| 192 | Fatty Acid Oxidation and Synthesis | 3- |
| | | Hydroxyhexadecanoylcarnitine |
| 193 | Fatty Acid Oxidation and Synthesis | Oleoylcarnitine |
| 194 | Fatty Acid Oxidation and Synthesis | 3-Hydroxyoleoylcarnitine |
| 195 | Fatty Acid Oxidation and Synthesis | Linoleylcarnitine |
| 196 | Fatty Acid Oxidation and Synthesis | 3-Hydroxylinoleylcarnitine |
| 197 | Fatty Acid Oxidation and Synthesis | Octadecandioylcarnitine |
| 198 | Fatty Acid Oxidation and Synthesis | O-succinylcarnitine |
| 199 | Fatty Acid Oxidation and Synthesis | 3-Hydroxyhexanoylcarnitine |
| 200 | Fatty Acid Oxidation and Synthesis | Adipoylcarnitine |
| 201 | Fatty Acid Oxidation and Synthesis | Octanoylcarnitine |
| 202 | Fatty Acid Oxidation and Synthesis | 2-Octenoylcarnitine |
| 203 | Fatty Acid Oxidation and Synthesis | Suberylcarnitine |
| 204 | Fatty Acid Oxidation and Synthesis | Adipic acid |
| 205 | Food Sources, Additives, Preservatives, Colorings, | Anserine |
| | and Dyes | |
| 206 | Food Sources, Additives, Preservatives, Colorings, | Methylcysteine |
| | and Dyes | |
| 207 | Food Sources, Additives, Preservatives, Colorings, | Red dye 40 |
| | and Dyes | |
| 208 | Food Sources, Additives, Preservatives, Colorings, | Dimethyl sulfone |
| | and Dyes | |
| 209 | GABA, Glutamate, Arginine, Ornithine, Proline | 1-Pyrroline-5-carboxylic acid |
| | Metabolism | |
| 210 | GABA, Glutamate, Arginine, Ornithine, Proline | Gamma-Aminobutyric acid |
| | Metabolism | |
| 211 | GABA, Glutamate, Arginine, Ornithine, Proline | Pyroglutamic acid |
| | Metabolism | |
| 212 | GABA, Glutamate, Arginine, Ornithine, Proline | Arginine_pos |
| | Metabolism | |
| 213 | GABA, Glutamate, Arginine, Ornithine, Proline | N-acetylornithine |
| | Metabolism | |
| 214 | GABA, Glutamate, Arginine, Ornithine, Proline | L-Proline |
| | Metabolism | |
| 215 | Gamma-Glutamyl and other Dipeptides | Gamma-glutamyl-Alanine |
| 216 | Gamma-Glutamyl and other Dipeptides | Gamma-glutamyl-Cysteine |
| 217 | Gamma-Glutamyl and other Dipeptides | Gamma-glutamyl-Isoleucine |
| 218 | Gamma-Glutamyl and other Dipeptides | Gamma-glutamyl-Leucine |
| 219 | Gamma-Glutamyl and other Dipeptides | Gamma-glutamyl-Valine |
| 220 | Gamma-Glutamyl and other Dipeptides | Glycylproline |
| 221 | Glycolipid Metabolism | GC (18:1/16:0) |
| 222 | Glycolipid Metabolism | GC (18:1/20:0) |
| 223 | Glycolipid Metabolism | GC (18:1/22:0) |
| 224 | Glycolipid Metabolism | GC (18:1/24:0) |
| 225 | Glycolipid Metabolism | GC (18:1/24:1) |
| 226 | Glycolipid Metabolism | THC 18:1/16:0 |
| 227 | Glycolipid Metabolism | THC 18:1/18:0 |
| 228 | Glycolipid Metabolism | THC 18:1/20:0 |
| 229 | Glycolipid Metabolism | THC 18:1/22:0 |
| 230 | Glycolipid Metabolism | THC 18:1/24:0 |
| 231 | Glycolipid Metabolism | THC 18:1/24:1 |
| 232 | Glycolysis, Gluconeogenesis, Galactose Metabolism | Glyceric acid 1,3-biphosphate |
| 233 | Glycolysis, Gluconeogenesis, Galactose Metabolism | 2-deoxyglucose-6-phosphate |
| 234 | Glycolysis, Gluconeogenesis, Galactose Metabolism | 2,3-Diphosphoglyceric acid |
| 235 | Glycolysis, Gluconeogenesis, Galactose Metabolism | Galactose 1-phosphate |
| 236 | Glycolysis, Gluconeogenesis, Galactose Metabolism | Fructose 1-phosphate |
| 237 | Glycolysis, Gluconeogenesis, Galactose Metabolism | Fructose 1,6-bisphosphate |
| 238 | Glycolysis, Gluconeogenesis, Galactose Metabolism | Fructose 6-phosphate |
| 239 | Glycolysis, Gluconeogenesis, Galactose Metabolism | Glucose 1-phosphate |
| 240 | Glycolysis, Gluconeogenesis, Galactose Metabolism | Dihydroxyacetone phosphate |
| 241 | Glycolysis, Gluconeogenesis, Galactose Metabolism | Glucose 6-phosphate |
| 242 | Glycolysis, Gluconeogenesis, Galactose Metabolism | Glyceraldehyde |
| 243 | Glycolysis, Gluconeogenesis, Galactose Metabolism | D-Glyceraldehyde 3- |
| | | phosphate |
| 244 | Glycolysis, Gluconeogenesis, Galactose Metabolism | 3-Phosphoglyceric acid |
| 245 | Glycolysis, Gluconeogenesis, Galactose Metabolism | Glyceric acid |
| 246 | Glycolysis, Gluconeogenesis, Galactose Metabolism | Glycerol |
| 247 | Glycolysis, Gluconeogenesis, Galactose Metabolism | Glycerol-3-phosphate |
| 248 | Glycolysis, Gluconeogenesis, Galactose Metabolism | Hexose_Pool_fru_glc-D |
| 249 | Glycolysis, Gluconeogenesis, Galactose Metabolism | L-Lactic acid |
| 250 | Glycolysis, Gluconeogenesis, Galactose Metabolism | Phosphoenolpyruvate |
| 251 | Gonadal Steroids | Testosterone |
| 252 | Gonadal Steroids | Dehydroisoandrosterone 3- |
| | | sulfate |
| 253 | Gonadal Steroids | Estradiol |
| 254 | Gonadal Steroids | Estriol |
| 255 | Gonadal Steroids | 17alpha-Hydroxyprogesterone |
| 256 | Gonadal Steroids | Progesterone |
| 257 | Gonadal Steroids | Testosterone benzoate |
| 258 | Gonadal Steroids | 17-alpha-Methyltestosterone |
| 259 | Heme and Porphyrin Metabolism | Bilirubin |
| 260 | Heme and Porphyrin Metabolism | Hemin-a |
| 261 | Heme and Porphyrin Metabolism | Hemin-b |
| 262 | Heme and Porphyrin Metabolism | Protoporphyrin IX |
| 263 | Heme and Porphyrin Metabolism | Coproporphyrin I |
| 264 | Histidine, Histamine Metabolism Metabolism | 1-Methylhistamine |
| 265 | Histidine, Histamine Metabolism Metabolism | 1-Methylhistidine |
| 266 | Histidine, Histamine Metabolism Metabolism | Carnosine |
| 267 | Histidine, Histamine Metabolism Metabolism | Histamine |
| 268 | Histidine, Histamine Metabolism Metabolism | L-Histidine |
| 269 | Isoleucine, Valine, Threonine, or Methionine | 2-Methylcitric acid |
| | Metabolism | |
| 270 | Isoleucine, Valine, Threonine, or Methionine | Tiglylglycine |
| | Metabolism | |
| 271 | Isoleucine, Valine, Threonine, or Methionine | Propionic acid |
| | Metabolism | |
| 272 | Isoleucine, Valine, Threonine, or Methionine | Propionyl-CoA |
| | Metabolism | |
| 273 | Isoleucine, Valine, Threonine, or Methionine | Propionylcarnitine |
| | Metabolism | |
| 274 | Ketone Body Metabolism | Acetoacetic acid |
| 275 | Ketone Body Metabolism | Acetoacetyl-CoA |
| 276 | Krebs Cycle | Citramalic acid |
| 277 | Krebs Cycle | 2-Hydroxyglutarate |
| 278 | Krebs Cycle | Acetic acid |
| 279 | Krebs Cycle | Acetyl-CoA |
| 280 | Krebs Cycle | Oxoglutaric acid |
| 281 | Krebs Cycle | cis-aconitic acid |
| 282 | Krebs Cycle | Citraconic acid |
| 283 | Krebs Cycle | Citric acid |
| 284 | Krebs Cycle | Coenzyme A_neg |
| 285 | Krebs Cycle | Coenzyme A_pos |
| 286 | Krebs Cycle | Dephospho-CoA |
| 287 | Krebs Cycle | Fumaric acid |
| 288 | Krebs Cycle | Isocitric acid |
| 289 | Krebs Cycle | Malic acid |
| 290 | Krebs Cycle | Oxaloacetic acid |
| 291 | Krebs Cycle | Pyruvic acid |
| 292 | Krebs Cycle | Succinic acid |
| 293 | Krebs Cycle | Succinyl-CoA |
| 294 | Lysine Metabolism | Aminoadipic acid |
| 295 | Lysine Metabolism | L-Lysine |
| 296 | Lysine Metabolism | Saccharopine |
| 297 | Microbiome Metabolism | 2-Aminoisobutyric acid |
| 298 | Microbiome Metabolism | 2-Pyrocatechuic acid |
| 299 | Microbiome Metabolism | 3-Hydroxyanthranilic acid |
| 300 | Microbiome Metabolism | 3-methylphenylacetic acid |
| 301 | Microbiome Metabolism | 4-Hydroxybenzoic acid |
| 302 | Microbiome Metabolism | 4-hydroxyphenyllactic acid |
| 303 | Microbiome Metabolism | 4-Hydroxyphenylpyruvic acid |
| 304 | Microbiome Metabolism | 4-Nitrophenol |
| 305 | Microbiome Metabolism | 5-adenylsulfate |
| 306 | Microbiome Metabolism | 2-Aminobenzoic acid |
| 307 | Microbiome Metabolism | Benzoic acid |
| 308 | Microbiome Metabolism | Butyryl-CoA |
| 309 | Microbiome Metabolism | Butyrylcarnitine |
| 310 | Microbiome Metabolism | Cellobiose |
| 311 | Microbiome Metabolism | Pipecolic acid |
| 312 | Microbiome Metabolism | Gluconic acid |
| 313 | Microbiome Metabolism | Hippuric acid |
| 314 | Microbiome Metabolism | Imidazole |
| 315 | Microbiome Metabolism | Imidazoleacetic acid |
| 316 | Microbiome Metabolism | Indole |
| 317 | Microbiome Metabolism | Indole-3-carboxylic acid |
| 318 | Microbiome Metabolism | Indoleacrylic acid |
| 319 | Microbiome Metabolism | L-Histidinol |
| 320 | Microbiome Metabolism | N-acetylserine |
| 321 | Microbiome Metabolism | O-acetylserine |
| 322 | Microbiome Metabolism | p-Aminobenzoic acid |
| 323 | Microbiome Metabolism | p-Hydroxybenzoate |
| 324 | Microbiome Metabolism | p-Hydroxyphenylacetic acid |
| 325 | Microbiome Metabolism | Phenyllactate |
| 326 | Microbiome Metabolism | Phenylpropiolic acid |
| 327 | Microbiome Metabolism | Phenylpyruvic acid |
| 328 | Microbiome Metabolism | Prephenate |
| 329 | Microbiome Metabolism | Shikimate |
| 330 | Microbiome Metabolism | Shikimate-3-phosphate |
| 331 | Microbiome Metabolism | Xanthosine |
| 332 | Microbiome Metabolism | Xanthylic acid |
| 333 | Nitric Oxide, Superoxide, Peroxide Metabolism | 3-Nitrotyrosine |
| 334 | Nitric Oxide, Superoxide, Peroxide Metabolism | 8-Hydroxy-deoxyguanosine |
| 335 | Nitric Oxide, Superoxide, Peroxide Metabolism | 8-hydroxy guanosine_neg |
| 336 | Nitric Oxide, Superoxide, Peroxide Metabolism | 8-hydroxy guanosine_pos |
| 337 | Nitric Oxide, Superoxide, Peroxide Metabolism | Lipoic acid |
| 338 | Nitric Oxide, Superoxide, Peroxide Metabolism | Azelylcarnitine |
| 339 | Nitric Oxide, Superoxide, Peroxide Metabolism | Azelaic acid |
| 340 | Nitric Oxide, Superoxide, Peroxide Metabolism | Malondialdehyde |
| 341 | Nitric Oxide, Superoxide, Peroxide Metabolism | 4-Hydroxynonenal |
| 342 | Non-glucose Carbohydrate and Amino-Sugar | Glucosamine 6-phosphate |
| | Metabolism | |
| 343 | Non-glucose Carbohydrate and Amino-Sugar | Mannose 6-phosphate |
| | Metabolism | |
| 344 | Non-glucose Carbohydrate and Amino-Sugar | Glucosamine |
| | Metabolism | |
| 345 | Non-glucose Carbohydrate and Amino-Sugar | Glucosamine-1-Phosphate |
| | Metabolism | |
| 346 | Non-glucose Carbohydrate and Amino-Sugar | Glucosamine-6-Phosphate |
| | Metabolism | |
| 347 | Non-glucose Carbohydrate and Amino-Sugar | Myoinositol |
| | Metabolism | |
| 348 | Non-glucose Carbohydrate and Amino-Sugar | N-acetyl-glucosamine |
| | Metabolism | 1-phosphate |
| 349 | Non-glucose Carbohydrate and Amino-Sugar | Sucrose |
| | Metabolism | |
| 350 | Non-glucose Carbohydrate and Amino-Sugar | Trehalose-6-Phosphate |
| | Metabolism | |
| 351 | Non-glucose Carbohydrate and Amino-Sugar | Aspartylglycosamine |
| | Metabolism | |
| 352 | OTC and Prescription Pharmaceutical Metabolism | Cotinine |
| 353 | OTC and Prescription Pharmaceutical Metabolism | Quinine hydrochloride |
| 354 | OTC and Prescription Pharmaceutical Metabolism | Salicyluric acid |
| 355 | OTC and Prescription Pharmaceutical Metabolism | Sodium dichloroacetate |
| 356 | OTC and Prescription Pharmaceutical Metabolism | Suramin-a |
| 357 | OTC and Prescription Pharmaceutical Metabolism | Suramin-b |
| 358 | OTC and Prescription Pharmaceutical Metabolism | Suramin-c |
| 359 | OTC and Prescription Pharmaceutical Metabolism | Suramin-d |
| 360 | OTC and Prescription Pharmaceutical Metabolism | Prednisolone acetate |
| 361 | OTC and Prescription Pharmaceutical Metabolism | Atorvastatin_neg |
| 362 | OTC and Prescription Pharmaceutical Metabolism | Bezafibrate |
| 363 | OTC and Prescription Pharmaceutical Metabolism | Cortisone |
| 364 | OTC and Prescription Pharmaceutical Metabolism | Dexamethasone 21-Acetate |
| 365 | OTC and Prescription Pharmaceutical Metabolism | Hydrocortisone 21-hydrogen |
| | | succinate |
| 366 | OTC and Prescription Pharmaceutical Metabolism | Norfluoxetine |
| 367 | OTC and Prescription Pharmaceutical Metabolism | Citalopram |
| 368 | OTC and Prescription Pharmaceutical Metabolism | Chlorpromazine |
| 369 | OTC and Prescription Pharmaceutical Metabolism | Fluoxetine |
| 370 | OTC and Prescription Pharmaceutical Metabolism | Venlafaxine |
| 371 | OTC and Prescription Pharmaceutical Metabolism | Desipramine |
| 372 | OTC and Prescription Pharmaceutical Metabolism | Phencyclidine |
| 373 | OTC and Prescription Pharmaceutical Metabolism | Baclofen |
| 374 | OTC and Prescription Pharmaceutical Metabolism | Chlordiazepoxide |
| 375 | OTC and Prescription Pharmaceutical Metabolism | 9-Hydroxyrisperidone |
| 376 | OTC and Prescription Pharmaceutical Metabolism | Acepromazine |
| 377 | OTC and Prescription Pharmaceutical Metabolism | Amisulpride |
| 378 | OTC and Prescription Pharmaceutical Metabolism | Amoxapine |
| 379 | OTC and Prescription Pharmaceutical Metabolism | Bidesmethylcitalopram |
| 380 | OTC and Prescription Pharmaceutical Metabolism | Caffeine |
| 381 | OTC and Prescription Pharmaceutical Metabolism | Cerivastatin |
| 382 | OTC and Prescription Pharmaceutical Metabolism | Chloroquine |
| 383 | OTC and Prescription Pharmaceutical Metabolism | Chlorpromazine Sulfoxide |
| 384 | OTC and Prescription Pharmaceutical Metabolism | Desmethylcitalopram |
| 385 | OTC and Prescription Pharmaceutical Metabolism | Desoxycortone 21-(3- |
| | | phenylpropionate) |
| 386 | OTC and Prescription Pharmaceutical Metabolism | Desoxycortone enantate |
| 387 | OTC and Prescription Pharmaceutical Metabolism | Dexamethasone 21- |
| | | isonicotinate |
| 388 | OTC and Prescription Pharmaceutical Metabolism | Etofibrate |
| 389 | OTC and Prescription Pharmaceutical Metabolism | Felbamate |
| 390 | OTC and Prescription Pharmaceutical Metabolism | Fenofibrate |
| 391 | OTC and Prescription Pharmaceutical Metabolism | Hydrocortisone 21-acetate |
| 392 | OTC and Prescription Pharmaceutical Metabolism | Hydrocortisone buteprate |
| 393 | OTC and Prescription Pharmaceutical Metabolism | Hydroxychlorquine |
| 394 | OTC and Prescription Pharmaceutical Metabolism | Imipramine |
| 395 | OTC and Prescription Pharmaceutical Metabolism | Levodopa |
| 396 | OTC and Prescription Pharmaceutical Metabolism | Lofepramine |
| 397 | OTC and Prescription Pharmaceutical Metabolism | Lovastatin |
| 398 | OTC and Prescription Pharmaceutical Metabolism | Loxapine |
| 399 | OTC and Prescription Pharmaceutical Metabolism | Melperone |
| 400 | OTC and Prescription Pharmaceutical Metabolism | Metformin |
| 401 | OTC and Prescription Pharmaceutical Metabolism | Methyldopa |
| 402 | OTC and Prescription Pharmaceutical Metabolism | Methylprednisolone |
| 403 | OTC and Prescription Pharmaceutical Metabolism | Methylscopolamine |
| 404 | OTC and Prescription Pharmaceutical Metabolism | Metoprolol |
| 405 | OTC and Prescription Pharmaceutical Metabolism | Diazepam |
| 406 | OTC and Prescription Pharmaceutical Metabolism | Trimipramine |
| 407 | OTC and Prescription Pharmaceutical Metabolism | Prazosin |
| 408 | OTC and Prescription Pharmaceutical Metabolism | Trazodone |
| 409 | OTC and Prescription Pharmaceutical Metabolism | Haloperidol |
| 410 | OTC and Prescription Pharmaceutical Metabolism | Fluphenazine |
| 411 | OTC and Prescription Pharmaceutical Metabolism | Levodopa |
| 412 | OTC and Prescription Pharmaceutical Metabolism | Methyldopa |
| 413 | OTC and Prescription Pharmaceutical Metabolism | Methylprednisolone |
| 414 | OTC and Prescription Pharmaceutical Metabolism | Prednisolone |
| 415 | OTC and Prescription Pharmaceutical Metabolism | Prednisone |
| 416 | OTC and Prescription Pharmaceutical Metabolism | Methylprednisolone acetate |
| 417 | OTC and Prescription Pharmaceutical Metabolism | Nefazodone |
| 418 | OTC and Prescription Pharmaceutical Metabolism | Olanzapine |
| 419 | OTC and Prescription Pharmaceutical Metabolism | Paroxetine |
| 420 | OTC and Prescription Pharmaceutical Metabolism | Phenothiazine |
| 421 | OTC and Prescription Pharmaceutical Metabolism | Phenytoin |
| 422 | OTC and Prescription Pharmaceutical Metabolism | Pioglitazone |
| 423 | OTC and Prescription Pharmaceutical Metabolism | Promethazine |
| 424 | OTC and Prescription Pharmaceutical Metabolism | Protriptyline |
| 425 | OTC and Prescription Pharmaceutical Metabolism | Quetiapine |
| 426 | OTC and Prescription Pharmaceutical Metabolism | Rosiglitazone |
| 427 | OTC and Prescription Pharmaceutical Metabolism | Scopolamine |
| 428 | OTC and Prescription Pharmaceutical Metabolism | Sertindole |
| 429 | OTC and Prescription Pharmaceutical Metabolism | Sertraline |
| 430 | OTC and Prescription Pharmaceutical Metabolism | Sildenafil |
| 431 | OTC and Prescription Pharmaceutical Metabolism | Simvastatin |
| 432 | OTC and Prescription Pharmaceutical Metabolism | Thiothixene |
| 433 | OTC and Prescription Pharmaceutical Metabolism | Zotepine |
| 434 | OTC and Prescription Pharmaceutical Metabolism | Lorazepam |
| 435 | OTC and Prescription Pharmaceutical Metabolism | Gabapentin |
| 436 | OTC and Prescription Pharmaceutical Metabolism | Propranolol |
| 437 | OTC and Prescription Pharmaceutical Metabolism | Amitriptylin |
| 438 | OTC and Prescription Pharmaceutical Metabolism | Risperidone |
| 439 | OTC and Prescription Pharmaceutical Metabolism | Midazolam |
| 440 | OTC and Prescription Pharmaceutical Metabolism | Zolpidem |
| 441 | OTC and Prescription Pharmaceutical Metabolism | Clozapine |
| 442 | OTC and Prescription Pharmaceutical Metabolism | Doxepin |
| 443 | OTC and Prescription Pharmaceutical Metabolism | Mirtazapine |
| 444 | OTC and Prescription Pharmaceutical Metabolism | Nortriptyline |
| 445 | OTC and Prescription Pharmaceutical Metabolism | Allopurinol |
| 446 | OTC and Prescription Pharmaceutical Metabolism | Clonidine |
| 447 | OTC and Prescription Pharmaceutical Metabolism | Carbamazepine |
| 448 | OTC and Prescription Pharmaceutical Metabolism | Aripiprazole |
| 449 | OTC and Prescription Pharmaceutical Metabolism | Thioridazine |
| 450 | Oxalate Metabolism | Glycolic acid |
| 451 | Oxalate Metabolism | Glyoxylic acid |
| 452 | Oxalate Metabolism | Oxalic acid |
| 453 | Pentose Phosphate, Gluconate Metabolism | 2-Keto-L-gluconate |
| 454 | Pentose Phosphate, Gluconate Metabolism | 6-Phosphogluconic acid |
| 455 | Pentose Phosphate, Gluconate Metabolism | Glucaric acid |
| 456 | Pentose Phosphate, Gluconate Metabolism | D-Ribose 5-phosphate |
| 457 | Pentose Phosphate, Gluconate Metabolism | Erythrose-4-phosphate |
| 458 | Pentose Phosphate, Gluconate Metabolism | Gluconolactone |
| 459 | Pentose Phosphate, Gluconate Metabolism | Glutaconic acid |
| 460 | Pentose Phosphate, Gluconate Metabolism | Octulose-1,8-bisphosphate |
| 461 | Pentose Phosphate, Gluconate Metabolism | Octulose-monophosphate |
| 462 | Pentose Phosphate, Gluconate Metabolism | Sedoheptulose 1,7- |
| | | bisphosphate |
| 463 | Pentose Phosphate, Gluconate Metabolism | Sedoheptulose 7-phosphate |
| 464 | Phosphate and Pyrophosphate Metabolism | Pyrophosphate |
| 465 | Phospholipid Metabolism | DL-O-Phosphoserine |
| 466 | Phospholipid Metabolism | BMP (16:0/16:0) |
| 467 | Phospholipid Metabolism | BMP (18:1/16:0) |
| 468 | Phospholipid Metabolism | BMP (18:1/16:1) |
| 469 | Phospholipid Metabolism | BMP (18:1/18:0) |
| 470 | Phospholipid Metabolism | BMP (18:1/18:1) |
| 471 | Phospholipid Metabolism | BMP (18:1/18:2) |
| 472 | Phospholipid Metabolism | BMP (18:1/20:4) |
| 473 | Phospholipid Metabolism | BMP (18:1/22:5) |
| 474 | Phospholipid Metabolism | BMP (18:1/22:6) |
| 475 | Phospholipid Metabolism | BMP (20:4/22:6) |
| 476 | Phospholipid Metabolism | BMP (22:5/22:6) |
| 477 | Phospholipid Metabolism | BMP (22:6/22:6) |
| 478 | Phospholipid Metabolism | Ethanolamine |
| 479 | Phospholipid Metabolism | Glycerophosphocholine |
| 480 | Phospholipid Metabolism | LysoPC (16:0) |
| 481 | Phospholipid Metabolism | LysoPC (18:0) |
| 482 | Phospholipid Metabolism | LysoPC (22:0) |
| 483 | Phospholipid Metabolism | N-oleoylethanolamine |
| 484 | Phospholipid Metabolism | PA (12:0/16:0) |
| 485 | Phospholipid Metabolism | PA (12:0/16:1) |
| 486 | Phospholipid Metabolism | PA (16:1/16:1) |
| 487 | Phospholipid Metabolism | PA (16:1/18:1) |
| 488 | Phospholipid Metabolism | PA (18:0/16:1)_neg |
| 489 | Phospholipid Metabolism | PA (18:0/16:1)_pos |
| 490 | Phospholipid Metabolism | PA (18:0/18:1)_neg |
| 491 | Phospholipid Metabolism | PA (18:0/18:1)_pos |
| 492 | Phospholipid Metabolism | PA (30:0) |
| 493 | Phospholipid Metabolism | PA (30:1) |
| 494 | Phospholipid Metabolism | PA (32:0)_neg |
| 495 | Phospholipid Metabolism | PA (32:0)_pos |
| 496 | Phospholipid Metabolism | PA (32:1) |
| 497 | Phospholipid Metabolism | PA (36:2)_neg |
| 498 | Phospholipid Metabolism | PA (36:2)_pos |
| 499 | Phospholipid Metabolism | Palmitoylethanolamide |
| 500 | Phospholipid Metabolism | PC (14:0/18:0)-Na |
| 501 | Phospholipid Metabolism | PC (16:0/18:1) |
| 502 | Phospholipid Metabolism | PC (16:0/18:1)-Na |
| 503 | Phospholipid Metabolism | PC (16:0/18:2) |
| 504 | Phospholipid Metabolism | PC (16:0/20:4) |
| 505 | Phospholipid Metabolism | PC (16:0/22:6) |
| 506 | Phospholipid Metabolism | PC (18:0/18:2) |
| 507 | Phospholipid Metabolism | PC (18:0/18:2)-Na |
| 508 | Phospholipid Metabolism | PC (18:0/20:3) |
| 509 | Phospholipid Metabolism | PC (18:3/22:4) |
| 510 | Phospholipid Metabolism | PC (20:4/P-16:0) |
| 511 | Phospholipid Metabolism | PC (20:5/P-16:0) |
| 512 | Phospholipid Metabolism | LysoPC(22:0) |
| 513 | Phospholipid Metabolism | PC (22:1) |
| 514 | Phospholipid Metabolism | PC (22:6/P-18:0) |
| 515 | Phospholipid Metabolism | LysoPC(24:0) |
| 516 | Phospholipid Metabolism | PC (24:0/P-18:0) |
| 517 | Phospholipid Metabolism | LysoPC(24:1(15Z)) |
| 518 | Phospholipid Metabolism | PC (26:0) |
| 519 | Phospholipid Metabolism | PC (26:1) |
| 520 | Phospholipid Metabolism | PC (28:0) |
| 521 | Phospholipid Metabolism | PC (28:1) |
| 522 | Phospholipid Metabolism | PC (28:2) |
| 523 | Phospholipid Metabolism | PC (30:0) |
| 524 | Phospholipid Metabolism | PC (30:1) |
| 525 | Phospholipid Metabolism | PC (30:2) |
| 526 | Phospholipid Metabolism | PC (16:0/16:0) |
| 527 | Phospholipid Metabolism | PC (32:1) |
| 528 | Phospholipid Metabolism | PC (32:2) |
| 529 | Phospholipid Metabolism | PC (34:1) |
| 530 | Phospholipid Metabolism | PC (34:2) |
| 531 | Phospholipid Metabolism | PC (36:0) |
| 532 | Phospholipid Metabolism | PC (36:1) |
| 533 | Phospholipid Metabolism | PC(18:1(9Z)/18:1(9Z)) |
| 534 | Phospholipid Metabolism | PC (38:5) |
| 535 | Phospholipid Metabolism | PC (40:6) |
| 536 | Phospholipid Metabolism | PE(20:4/P-18:1) |
| 537 | Phospholipid Metabolism | PE (28:0) |
| 538 | Phospholipid Metabolism | PE (28:1) |
| 539 | Phospholipid Metabolism | PE (30:0) |
| 540 | Phospholipid Metabolism | PE (30:1) |
| 541 | Phospholipid Metabolism | PE (30:2) |
| 542 | Phospholipid Metabolism | PE (32:1) |
| 543 | Phospholipid Metabolism | PE (32:2) |
| 544 | Phospholipid Metabolism | PE (34:1) |
| 545 | Phospholipid Metabolism | PE (34:2) |
| 546 | Phospholipid Metabolism | PE (36:1) |
| 547 | Phospholipid Metabolism | PE (36:2) |
| 548 | Phospholipid Metabolism | PE (36:3) |
| 549 | Phospholipid Metabolism | PE (38:4) |
| 550 | Phospholipid Metabolism | PE (38:5) |
| 551 | Phospholipid Metabolism | PG (32:1)_neg |
| 552 | Phospholipid Metabolism | PG (32:1)_pos |
| 553 | Phospholipid Metabolism | PG (32:2) |
| 554 | Phospholipid Metabolism | PG (34:1)_neg |
| 555 | Phospholipid Metabolism | PG (34:1)_pos |
| 556 | Phospholipid Metabolism | PG (34:2)_neg |
| 557 | Phospholipid Metabolism | PG (34:2)_pos |
| 558 | Phospholipid Metabolism | PG (36:1) |
| 559 | Phospholipid Metabolism | PG (36:2) |
| 560 | Phospholipid Metabolism | PG (36:3) |
| 561 | Phospholipid Metabolism | PG (38:4) |
| 562 | Phospholipid Metabolism | PG (40:8) |
| 563 | Phospholipid Metabolism | PG (44:12) |
| 564 | Phospholipid Metabolism | PG(16:0/16:0) |
| 565 | Phospholipid Metabolism | O-Phosphoethanolamine |
| 566 | Phospholipid Metabolism | Phosphorylcholine |
| 567 | Phospholipid Metabolism | PI (26:0) |
| 568 | Phospholipid Metabolism | PI (26:1) |
| 569 | Phospholipid Metabolism | PI (28:0) |
| 570 | Phospholipid Metabolism | PI (28:1) |
| 571 | Phospholipid Metabolism | PI (30:0) |
| 572 | Phospholipid Metabolism | PI (30:1) |
| 573 | Phospholipid Metabolism | PI (30:2) |
| 574 | Phospholipid Metabolism | PI(16:0/16:0) |
| 575 | Phospholipid Metabolism | PI (32:1) |
| 576 | Phospholipid Metabolism | PI (32:2) |
| 577 | Phospholipid Metabolism | PI (34:0) |
| 578 | Phospholipid Metabolism | PI (34:1) |
| 579 | Phospholipid Metabolism | PI (34:2) |
| 580 | Phospholipid Metabolism | PI (36:0) |
| 581 | Phospholipid Metabolism | PI (36:1) |
| 582 | Phospholipid Metabolism | PI (36:2) |
| 583 | Phospholipid Metabolism | PI (36:4) |
| 584 | Phospholipid Metabolism | PI (38:3) |
| 585 | Phospholipid Metabolism | PI (38:4) |
| 586 | Phospholipid Metabolism | PI (38:5) |
| 587 | Phospholipid Metabolism | PI (40:5) |
| 588 | Phospholipid Metabolism | PS(16:0/16:0) |
| 589 | Phospholipid Metabolism | PS (32:1) |
| 590 | Phospholipid Metabolism | PS (32:2) |
| 591 | Phospholipid Metabolism | PS (34:1) |
| 592 | Phospholipid Metabolism | PS (34:2) |
| 593 | Phospholipid Metabolism | PS (36:0) |
| 594 | Phospholipid Metabolism | PS(18:0/18:1(9Z)) |
| 595 | Phospholipid Metabolism | PS (36:2) |
| 596 | Phospholipid Metabolism | PS(18:0/20:4(8Z,11Z,14Z,17Z)) |
| 597 | Phytanic, Branch, Odd Chain Fatty Acid Metabolism | 2-Isopropylmalic acid |
| 598 | Phytanic, Branch, Odd Chain Fatty Acid Metabolism | Pimelylcarnitine |
| 599 | Phytonutrients, Bioactive Botanical Metabolites | Curcumin |
| 600 | Phytonutrients, Bioactive Botanical Metabolites | Epicatechin |
| 601 | Phytonutrients, Bioactive Botanical Metabolites | Genistein |
| 602 | Phytonutrients, Bioactive Botanical Metabolites | Hyoscyamine |
| 603 | Plasmalogen Metabolism | p16:0/20:4/PEtn |
| 604 | Plasmalogen Metabolism | p18:0/20:4/PEtn |
| 605 | Plasmalogen Metabolism | p18:0/22:6/PEtn |
| 606 | Polyamine Metabolism | 5-Methylthioadenosine |
| 607 | Polyamine Metabolism | Agmatine |
| 608 | Polyamine Metabolism | Agmatine sulfate |
| 609 | Polyamine Metabolism | N-acetylputrescine |
| 610 | Polyamine Metabolism | Putrescine |
| 611 | Polyamine Metabolism | Spermidine |
| 612 | Polyamine Metabolism | Spermine |
| 613 | Polyamine Metabolism | Tyramine |
| 614 | Polyamine Metabolism | Cadaverine |
| 615 | Purine Metabolism | 1-Methyladenosine |
| 616 | Purine Metabolism | Cyclic GMP |
| 617 | Purine Metabolism | 7-Methylguanosine |
| 618 | Purine Metabolism | Adenine |
| 619 | Purine Metabolism | Adenosine |
| 620 | Purine Metabolism | Adenylsuccinic acid |
| 621 | Purine Metabolism | ADP |
| 622 | Purine Metabolism | ADP-glucose |
| 623 | Purine Metabolism | AICAR_neg |
| 624 | Purine Metabolism | AICAR_pos |
| 625 | Purine Metabolism | Allantoic acid |
| 626 | Purine Metabolism | Allantoin |
| 627 | Purine Metabolism | Adenosine |
| | | monophosphate_neg |
| 628 | Purine Metabolism | Adenosine |
| | | monophosphate_pos |
| 629 | Purine Metabolism | Adenosine triphosphate |
| 630 | Purine Metabolism | Cyclic AMP |
| 631 | Purine Metabolism | dADP |
| 632 | Purine Metabolism | Deoxyadenosine |
| | | monophosphate |
| 633 | Purine Metabolism | dATP |
| 634 | Purine Metabolism | Deoxyadenosine |
| 635 | Purine Metabolism | Deoxyguanosine |
| 636 | Purine Metabolism | Deoxyinosine_neg |
| 637 | Purine Metabolism | Deoxyinosine_pos |
| 638 | Purine Metabolism | Deoxyribose-phosphate |
| 639 | Purine Metabolism | dGDP |
| 640 | Purine Metabolism | 2-Deoxyguanosine 5- |
| | | monophosphate |
| 641 | Purine Metabolism | dGTP |
| 642 | Purine Metabolism | dIMP |
| 643 | Purine Metabolism | 2-Deoxyinosine triphosphate |
| 644 | Purine Metabolism | Guanosine diphosphate |
| 645 | Purine Metabolism | Guanosine monophosphate |
| 646 | Purine Metabolism | Guanosine triphosphate |
| 647 | Purine Metabolism | Guanine |
| 648 | Purine Metabolism | GDP |
| 649 | Purine Metabolism | Guanosine_neg |
| 650 | Purine Metabolism | Guanosine_pos |
| 651 | Purine Metabolism | Hypoxanthine_neg |
| 652 | Purine Metabolism | Hypoxanthine_pos |
| 653 | Purine Metabolism | IDP |
| 654 | Purine Metabolism | Inosinic acid |
| 655 | Purine Metabolism | Inosine_neg |
| 656 | Purine Metabolism | Inosine_pos |
| 657 | Purine Metabolism | Inosine triphosphate |
| 658 | Purine Metabolism | Phosphoribosyl pyrophosphate |
| 659 | Purine Metabolism | Purine |
| 660 | Purine Metabolism | Uric acid |
| 661 | Purine Metabolism | Xanthine_neg |
| 662 | Purine Metabolism | Xanthine_pos |
| 663 | Purine Metabolism | ZMP |
| 664 | Pyrimidine Metabolism | 4,5-Dihydroorotic acid |
| 665 | Pyrimidine Metabolism | Ureidosuccinic acid |
| 666 | Pyrimidine Metabolism | Carbamoylphosphate |
| 667 | Pyrimidine Metabolism | CDP |
| 668 | Pyrimidine Metabolism | Citicoline |
| 669 | Pyrimidine Metabolism | CDP-Ethanolamine |
| 670 | Pyrimidine Metabolism | Cytidine monophosphate |
| 671 | Pyrimidine Metabolism | Cytidine triphosphate |
| 672 | Pyrimidine Metabolism | Cytidine |
| 673 | Pyrimidine Metabolism | Cytosine |
| 674 | Pyrimidine Metabolism | dCDP |
| 675 | Pyrimidine Metabolism | dCMP |
| 676 | Pyrimidine Metabolism | dCTP |
| 677 | Pyrimidine Metabolism | Deoxyuridine_neg |
| 678 | Pyrimidine Metabolism | Deoxyuridine_pos |
| 679 | Pyrimidine Metabolism | dTDP |
| 680 | Pyrimidine Metabolism | dTDP-D-glucose |
| 681 | Pyrimidine Metabolism | 5-Thymidylic acid_pos |
| 682 | Pyrimidine Metabolism | 5-Thymidylic acid_neg |
| 683 | Pyrimidine Metabolism | Thymidine 5-triphosphate |
| 684 | Pyrimidine Metabolism | dUMP |
| 685 | Pyrimidine Metabolism | Deoxyuridine triphosphate |
| 686 | Pyrimidine Metabolism | Orotic acid |
| 687 | Pyrimidine Metabolism | Orotidine-phosphate |
| 688 | Pyrimidine Metabolism | Thymidine |
| 689 | Pyrimidine Metabolism | Thymine |
| 690 | Pyrimidine Metabolism | Uridine 5-diphosphate |
| 691 | Pyrimidine Metabolism | Uridine diphosphate glucose |
| 692 | Pyrimidine Metabolism | Uridine diphosphate |
| | | glucuronic acid |
| 693 | Pyrimidine Metabolism | UDP-n-acetyl-D-glucosamine |
| 694 | Pyrimidine Metabolism | Uridine 5-monophosphate |
| 695 | Pyrimidine Metabolism | Uracil_neg |
| 696 | Pyrimidine Metabolism | Uracil_pos |
| 697 | Pyrimidine Metabolism | Ureidopropionic acid |
| 698 | Pyrimidine Metabolism | Uridine |
| 699 | Pyrimidine Metabolism | Uridine triphosphate |
| 700 | SAM, SAH, Methionine, Cysteine, Glutathione | 2-Oxo-4-methylthiobutanoic |
| | Metabolism | acid |
| 701 | SAM, SAH, Methionine, Cysteine, Glutathione | 2-Ketobutyric acid |
| | Metabolism | |
| 702 | SAM, SAH, Methionine, Cysteine, Glutathione | 3-Methylthiopropionic acid |
| | Metabolism | |
| 703 | SAM, SAH, Methionine, Cysteine, Glutathione | Creatinine |
| | Metabolism | |
| 704 | SAM, SAH, Methionine, Cysteine, Glutathione | L-Cystathionine |
| | Metabolism | |
| 705 | SAM, SAH, Methionine, Cysteine, Glutathione | Cysteamine |
| | Metabolism | |
| 706 | SAM, SAH, Methionine, Cysteine, Glutathione | Cysteine |
| | Metabolism | |
| 707 | SAM, SAH, Methionine, Cysteine, Glutathione | Dimethyl-L-arginine |
| | Metabolism | |
| 708 | SAM, SAH, Methionine, Cysteine, Glutathione | Dimethylglycine |
| | Metabolism | |
| 709 | SAM, SAH, Methionine, Cysteine, Glutathione | L-Homocysteic acid |
| | Metabolism | |
| 710 | SAM, SAH, Methionine, Cysteine, Glutathione | Homocysteine |
| | Metabolism | |
| 711 | SAM, SAH, Methionine, Cysteine, Glutathione | L-Homoserine |
| | Metabolism | |
| 712 | SAM, SAH, Methionine, Cysteine, Glutathione | L-cystine |
| | Metabolism | |
| 713 | SAM, SAH, Methionine, Cysteine, Glutathione | L-Methionine |
| | Metabolism | |
| 714 | SAM, SAH, Methionine, Cysteine, Glutathione | Methionine sulfoxide |
| | Metabolism | |
| 715 | SAM, SAH, Methionine, Cysteine, Glutathione | Oxidized glutathione |
| | Metabolism | |
| 716 | SAM, SAH, Methionine, Cysteine, Glutathione | Glutathione_neg |
| | Metabolism | |
| 717 | SAM, SAH, Methionine, Cysteine, Glutathione | Glutathione_pos |
| | Metabolism | |
| 718 | SAM, SAH, Methionine, Cysteine, Glutathione | S-adenosylhomocysteine_neg |
| | Metabolism | |
| 719 | SAM, SAH, Methionine, Cysteine, Glutathione | S-adenosylmethionine |
| | Metabolism | |
| 720 | SAM, SAH, Methionine, Cysteine, Glutathione | S-adenosylhomocysteine_pos |
| | Metabolism | |
| 721 | SAM, SAH, Methionine, Cysteine, Glutathione | Sarcosine |
| | Metabolism | |
| 722 | SAM, SAH, Methionine, Cysteine, Glutathione | Cysteineglutathione disulfide |
| | Metabolism | |
| 723 | SAM, SAH, Methionine, Cysteine, Glutathione | Cysteine-S-sulfate |
| | Metabolism | |
| 724 | Sphingolipid Metabolism | Ceramide (d18:1/12:0) |
| 725 | Sphingolipid Metabolism | Ceramide (d18:1/16:0 OH) |
| 726 | Sphingolipid Metabolism | Ceramide (d18:1/16:0) |
| 727 | Sphingolipid Metabolism | Ceramide (d18:1/16:1 OH) |
| 728 | Sphingolipid Metabolism | Ceramide (d18:1/16:1) |
| 729 | Sphingolipid Metabolism | Ceramide (d18:1/16:2 OH) |
| 730 | Sphingolipid Metabolism | Ceramide (d18:1/16:2) |
| 731 | Sphingolipid Metabolism | Ceramide (d18:1/18:0 OH) |
| 732 | Sphingolipid Metabolism | Ceramide (d18:1/18:0) |
| 733 | Sphingolipid Metabolism | Ceramide (d18:1/18:1 OH) |
| 734 | Sphingolipid Metabolism | Ceramide (d18:1/18:1) |
| 735 | Sphingolipid Metabolism | Ceramide (d18:1/18:2 OH) |
| 736 | Sphingolipid Metabolism | Ceramide (d18:1/18:2) |
| 737 | Sphingolipid Metabolism | Ceramide (d18:1/20:0 OH) |
| 738 | Sphingolipid Metabolism | Ceramide (d18:1/20:0) |
| 739 | Sphingolipid Metabolism | Ceramide (d18:1/20:1 OH) |
| 740 | Sphingolipid Metabolism | Ceramide (d18:1/20:1) |
| 741 | Sphingolipid Metabolism | Ceramide (d18:1/20:2 OH) |
| 742 | Sphingolipid Metabolism | Ceramide (d18:1/20:2) |
| 743 | Sphingolipid Metabolism | Ceramide (d18:1/22:0 OH) |
| 744 | Sphingolipid Metabolism | Ceramide (d18:1/22:0) |
| 745 | Sphingolipid Metabolism | Ceramide (d18:1/22:1) |
| 746 | Sphingolipid Metabolism | Ceramide (d18:1/22:2 OH) |
| 747 | Sphingolipid Metabolism | Ceramide (d18:1/22:2) |
| 748 | Sphingolipid Metabolism | Ceramide (d18:1/23:0) or |
| | | (d18:1/22:1 OH) |
| 749 | Sphingolipid Metabolism | Ceramide (d18:1/24:0 OH) |
| 750 | Sphingolipid Metabolism | Ceramide (d18:1/24:0) |
| 751 | Sphingolipid Metabolism | Ceramide (d18:1/24:1) |
| 752 | Sphingolipid Metabolism | Ceramide (d18:1/24:2 OH) |
| 753 | Sphingolipid Metabolism | Ceramide (d18:1/24:2) |
| 754 | Sphingolipid Metabolism | Ceramide (d18:1/25:0) |
| 755 | Sphingolipid Metabolism | Ceramide (d18:1/26:0 OH) |
| 756 | Sphingolipid Metabolism | Ceramide (d18:1/26:0) |
| 757 | Sphingolipid Metabolism | Ceramide (d18:1/26:1 OH) |
| 758 | Sphingolipid Metabolism | Ceramide (d18:1/26:1) |
| 759 | Sphingolipid Metabolism | Ceramide (d18:1/26:2 OH) |
| 760 | Sphingolipid Metabolism | Ceramide (d18:1/26:2) |
| 761 | Sphingolipid Metabolism | DHC (18:1/16:0) |
| 762 | Sphingolipid Metabolism | DHC (18:1/20:0) |
| 763 | Sphingolipid Metabolism | DHC (18:1/22:0) |
| 764 | Sphingolipid Metabolism | DHC (18:1/24:0) |
| 765 | Sphingolipid Metabolism | DHC (18:1/24:1) |
| 766 | Sphingolipid Metabolism | SM (d18:1/16:0) |
| 767 | Sphingolipid Metabolism | SM (d18:1/18:1(9Z)) |
| 768 | Sphingolipid Metabolism | SM (d18:1/22:1(13Z)) |
| 769 | Sphingolipid Metabolism | SM (d18:1/24:0) |
| 770 | Sphingolipid Metabolism | SM (d18:1/26:0) |
| 771 | Sphingolipid Metabolism | SM 16:0 OH |
| 772 | Sphingolipid Metabolism | SM (d18:1/16:1) |
| 773 | Sphingolipid Metabolism | SM 16:1 OH |
| 774 | Sphingolipid Metabolism | SM (d18:1/16:2) |
| 775 | Sphingolipid Metabolism | SM 16:2 OH |
| 776 | Sphingolipid Metabolism | SM 18:0 OH |
| 777 | Sphingolipid Metabolism | SM 18:1 OH |
| 778 | Sphingolipid Metabolism | SM (d18:1/18:2) |
| 779 | Sphingolipid Metabolism | SM 18:2 OH |
| 780 | Sphingolipid Metabolism | SM (d18:1/20:0) |
| 781 | Sphingolipid Metabolism | SM 20:0 OH |
| 782 | Sphingolipid Metabolism | SM 20:1 |
| 783 | Sphingolipid Metabolism | SM 20:1 OH |
| 784 | Sphingolipid Metabolism | SM (d18:1/20:2) |
| 785 | Sphingolipid Metabolism | SM 20:2 OH |
| 786 | Sphingolipid Metabolism | SM 22:0 OH |
| 787 | Sphingolipid Metabolism | SM (d18:1/22:2) |
| 788 | Sphingolipid Metabolism | SM 22:2 OH |
| 789 | Sphingolipid Metabolism | SM 23:0 or SM 22:1 OH |
| 790 | Sphingolipid Metabolism | SM 24:0 OH |
| 791 | Sphingolipid Metabolism | SM (d18:1/24:2) |
| 792 | Sphingolipid Metabolism | SM 24:2 OH |
| 793 | Sphingolipid Metabolism | SM 25:0 or C24:1 OH |
| 794 | Sphingolipid Metabolism | SM 26:0 OH |
| 795 | Sphingolipid Metabolism | SM (d18:1/26:1) |
| 796 | Sphingolipid Metabolism | SM 26:1 OH |
| 797 | Sphingolipid Metabolism | SM (d18:1/26:2) |
| 798 | Sphingolipid Metabolism | SM 26:2 OH |
| 799 | Sphingolipid Metabolism | SM (d18:1/18:0) |
| 800 | Sphingolipid Metabolism | SM (d18:1/22:0) |
| 801 | Sphingolipid Metabolism | SM( d18:1/24:1(15Z)) |
| 802 | Sphingolipid Metabolism | SM (d18:1/12:0) |
| 803 | Taurine, Hypotaurine Metabolism | Acetylphosphate |
| 804 | Taurine, Hypotaurine Metabolism Metabolism | Taurine |
| 805 | Thyroxine Metabolism | 3,5-Diiodothyronine |
| 806 | Tryptophan, Kynurenine, Serotonin, Melatonin | 5-Hydroxy-L-tryptophan |
| | Metabolism | |
| 807 | Tryptophan, Kynurenine, Serotonin, Melatonin | 5-Hydroxyindoleacetic |
| | Metabolism | acid_neg |
| 808 | Tryptophan, Kynurenine, Serotonin, Melatonin | 5-Hydroxyindoleacetic |
| | Metabolism | acid_pos |
| 809 | Tryptophan, Kynurenine, Serotonin, Melatonin | 5-Methoxytryptophan |
| | Metabolism | |
| 810 | Tryptophan, Kynurenine, Serotonin, Melatonin | Hydroxykynurenine |
| | Metabolism | |
| 811 | Tryptophan, Kynurenine, Serotonin, Melatonin | Kynurenic acid |
| | Metabolism | |
| 812 | Tryptophan, Kynurenine, Serotonin, Melatonin | L-Kynurenine |
| | Metabolism | |
| 813 | Tryptophan, Kynurenine, Serotonin, Melatonin | Melatonin |
| | Metabolism | |
| 814 | Tryptophan, Kynurenine, Serotonin, Melatonin | Quinolinic Acid |
| | Metabolism | |
| 815 | Tryptophan, Kynurenine, Serotonin, Melatonin | Serotonin |
| | Metabolism | |
| 816 | Tryptophan, Kynurenine, Serotonin, Melatonin | L-Tryptophan |
| | Metabolism | |
| 817 | Tyrosine and Phenylalanine Metabolism | Homogentisic acid |
| 818 | Tyrosine and Phenylalanine Metabolism | L-Phenylalanine |
| 819 | Tyrosine and Phenylalanine Metabolism | O-Phosphotyrosine |
| 820 | Tyrosine and Phenylalanine Metabolism | L-Tyrosine |
| 821 | Ubiquinone Metabolism | Coenzyme Q10 |
| 822 | Ubiquinone Metabolism | CoQ10H2 |
| 823 | Ubiquinone Metabolism | Coenzyme Q9 |
| 824 | Ubiquinone Metabolism | CoQ9H2 |
| 825 | Urea Cycle | Citrulline_neg |
| 826 | Urea Cycle | Citrulline_pos |
| 827 | Urea Cycle | Argininosuccinic acid |
| 828 | Urea Cycle | Ornithine |
| 829 | Urea Cycle | Urea |
| 830 | Very Long Chain Fatty Acid Oxidation | Tetracosanoic acid |
| 831 | Very Long Chain Fatty Acid Oxidation | Behenic acid |
| 832 | Very Long Chain Fatty Acid Oxidation | Hexacosanoic acid |
| 833 | Vitamin A (Retinol), Carotenoid Metabolism | B-Carotene |
| 834 | Vitamin A (Retinol), Carotenoid Metabolism | Retinol |
| 835 | Vitamin A (Retinol), Carotenoid Metabolism | Retinal |
| 836 | Vitamin B1 (Thiamine) Metabolism | Thiamine |
| 837 | Vitamin B1 (Thiamine) Metabolism | Thiamine monophosphate |
| 838 | Vitamin B1 (Thiamine) Metabolism | Thiamine pyrophosphate_neg |
| 839 | Vitamin B1 (Thiamine) Metabolism | Thiamine Pyrophosphate_pos |
| 840 | Vitamin B12 (Cobalamin) Metabolism | Cyanocobalamin |
| 841 | Vitamin B12 (Cobalamin) Metabolism | Methylcobalamin |
| 842 | Vitamin B12 Metabolism | Cobalamin |
| 843 | Vitamin B12 Metabolism | Methylmalonic acid |
| 844 | Vitamin B2 (Riboflavin) Metabolism | FAD |
| 845 | Vitamin B2 (Riboflavin) Metabolism | Flavone |
| 846 | Vitamin B2 (Riboflavin) Metabolism | FMN |
| 847 | Vitamin B2 (Riboflavin) Metabolism | Riboflavin |
| 848 | Vitamin B3 (Niacin, NAD+) Metabolism | 1-Methylnicotinamide |
| 849 | Vitamin B3 (Niacin, NAD+) Metabolism | NAD |
| 850 | Vitamin B3 (Niacin, NAD+) Metabolism | NADH |
| 851 | Vitamin B3 (Niacin, NAD+) Metabolism | NADP |
| 852 | Vitamin B3 (Niacin, NAD+) Metabolism | NADPH |
| 853 | Vitamin B3 (Niacin, NAD+) Metabolism | Niacinamide |
| 854 | Vitamin B3 (Niacin, NAD+) Metabolism | Nicotinic acid |
| 855 | Vitamin B3 (Niacin, NAD+) Metabolism | Nicotinamide N-oxide |
| 856 | Vitamin B5 (Pantothenate) Metabolism | Pantothenic acid |
| 857 | Vitamin B6 (Pyridoxine) Metabolism | Pyridoxal |
| 858 | Vitamin B6 (Pyridoxine) Metabolism | Pyridoxal 5-phosphate |
| 859 | Vitamin B6 (Pyridoxine) Metabolism | Pyridoxamine |
| 860 | Vitamin B6 (Pyridoxine) Metabolism | Pyridoxine |
| 861 | Vitamin B6 (Pyridoxine) Metabolism | Xanthurenic acid |
| 862 | Vitamin B6 (Pyridoxine) Metabolism | 4-Pyridoxic acid |
| 863 | Vitamin C (Ascorbate) Metabolism | Hydroxyproline |
| 864 | Vitamin C (Ascorbate) Metabolism | L-ascorbic acid |
| 865 | Vitamin D (Calciferol) Metabolism | 5,6-trans-25-Hydroxyvitamin |
| | | D3 |
| 866 | Vitamin D (Calciferol) Metabolism | Vitamin D3 |
| 867 | Vitamin E (Tocopherol) Metabolism | Alpha-Tocopherol |
| 868 | Vitamin K (Menaquinone) Metabolism | Vitamin K2 |

A number of embodiments have been described herein. Nevertheless, it will be understood that various modifications may be made without departing from the spirit and scope of this disclosure. Accordingly, other embodiments are within the scope of the following claims.

1. A method for whether a subject has or is at risk of having post-traumatic stress disorder (PTSD), the method comprising detecting an amount of each of a plurality of metabolites in a biological sample obtained from the subject by: HPLC, TLC, electrochemical analysis, mass spectroscopy, refractive index spectroscopy (RI), Ultra-Violet spectroscopy (UV), fluorescent analysis, gas chromatography (GC), radiochemical analysis, Near-InfraRed spectroscopy (Near-IR), Nuclear Magnetic Resonance spectroscopy (NMR), and/or Light Scattering analysis (LS),
said plurality of metabolites comprising at least eight (8) metabolites, each of said at least 8 metabolites being in a metabolic pathway selected from the group of pathways consisting of:
a phospholipid metabolic pathway;
a fatty acid oxidation and synthesis metabolic pathway;
a purine metabolic pathway;
a bioamine and neurotransmitter metabolic pathway;
a microbiome metabolic pathway;
a sphingolipid metabolic pathway;
a cholesterol, cortisol, non-gonadal steroid metabolic pathway;
a pyrimidine metabolic pathway;
a 3- and 4-carbon amino acid metabolic pathway;
a branch chain amino acid metabolic pathway;
a tryptophan, kynurenine, serotonin, melatonin metabolic pathway;
a tyrosine and phenylalanine metabolic pathway;
a SAM, SAH, methionine, cysteine, glutathione metabolic pathway;
an eicosanoid and resolvin metabolic pathway;
a pentose phosphate, gluconate metabolic pathway; and
a vitamin A, carotenoid metabolic pathway; and
determining, based on said amounts so detected, the presence or absence of an alteration in each of a plurality of the group of pathways.

2. The method of claim 1, wherein determination of the presence of an alteration in at least eight of the group of pathways indicates that the subject has or is at risk of developing PTSD.

3. The method of claim 1, further comprising generating a PTSD metabolomics profile from the plurality of metabolites comprising at least 8 metabolic pathways selected from the group consisting of:
a phospholipid metabolic pathway;
a fatty acid oxidation and synthesis metabolic pathway;
a purine metabolic pathway;
a bioamine and neurotransmitter metabolic pathway;
a microbiome metabolic pathway;
a sphingolipid metabolic pathway;
a cholesterol, cortisol, non-gonadal steroid metabolic pathway;
a pyrimidine metabolic pathway;
a 3- and 4-carbon amino acid metabolic pathway;
a branch chain amino acid metabolic pathway;
a tryptophan, kynurenine, serotonin, melatonin metabolic pathway;
a tyrosine and phenylalanine metabolic pathway;
a SAM, SAH, methionine, cysteine, glutathione metabolic pathway;
an eicosanoid and resolvin metabolic pathway;
a pentose phosphate, gluconate metabolic pathway; and
a vitamin A, carotenoid metabolic pathway;
comparing the PTSD metabolomics profile to a normal control PTSD metabolomics profile, wherein when at least one metabolite of the plurality of metabolites is aberrantly produced in at least 8 metabolic pathways compared to the control PTSD metabolomics pathway, the subject has or is at risk of having PTSD.

4. The method of claim 3, wherein the at least one metabolite comprises at least 2 metabolites in each of the at least 8 metabolic pathways.

5. The method of claim 3, wherein generating the PTSD metabolomics profile from the subject, comprises determining the metabolic activity of each of the following pathways:
(i) a phospholipid metabolic pathway;
(ii) a fatty acid oxidation and synthesis metabolic pathway;
(iii) a purine metabolic pathway;
(iv) a bioamine and neurotransmitter metabolic pathway;
(v) a microbiome metabolic pathway;
(vi) a sphingolipid metabolic pathway;
(vii) a cholesterol, cortisol, non-gonadal steroid metabolic pathway;
(viii) a pyrimidine metabolic pathway;
(ix) a 3- and 4-carbon amino acid metabolic pathway;
(x) a branch chain amino acid metabolic pathway;
(xi) a tryptophan, kynurenine, serotonin, melatonin metabolic pathway;
(xii) a tyrosine and phenylalanine metabolic pathway;
(xiii) a SAM, SAH, methionine, cysteine, glutathione metabolic pathway;
(xiv) an eicosanoid and resolvin metabolic pathway;
(xv) a pentose phosphate, gluconate metabolic pathway; and
(xvi) a vitamin A, carotenoid metabolic pathway,
comparing the PTSD metabolomics profile from the subject to a control PTSD metabolomics profile comprising the pathways of (i)-(xvi), wherein when at least 8 of the metabolic pathways in (i)-(xvi) have aberrant activity, the subject has or is at risk of having PTSD.

6. The method of claim 3, wherein the small molecule metabolite profile comprises metabolites selected from the group consisting of: 2-Octenoylcarnitine, Retinol, L-Tryptophan, Nicotinamide N-oxide, Alanine, L-Tyrosine, 3-Hydroxyanthranilic acid, N-Acetyl-L-aspartic acid, Sarcosine, N-Acetylaspartylglutamic acid, Methylcysteine, AICAR, SM(d18:1/12:0), Oleic acid, Docosahexaenoic acid, Glycocholic acid, Guanosine monophosphate, Cytidine, SM(d18:1/22:0 OH), Xanthine, Indoleacrylic acid, 7-ketocholesterol, 3-Hydroxyhexadecanoylcarnitine, Linoleic acid, Adenosine monophosphate, L-Serine, Pantothenic acid, Arachidonic Acid, PC(26:1), Uracil and any combination thereof.

7. The method of claim 6, wherein the small molecule metabolite profile further comprises metabolites selected from the group consisting of: PC(30:2), Hypoxanthine, 2-Keto-L-gluconate, Glutaconic acid, 5-HETE, PC(28:2), 3-Hydroxyhexadecenoylcamitine, Hydroxyproline, Dopamine, Myoinositol, 3-Hydroxylinoleylcamitine, PC(30:1), LysoPC(24:0), Indole, SM(d18:1/24:0), PC(28:1), L-Threonine, Mevalonic acid, SM(20:0 OH), Purine ring, 3-Hydroxyisobutyroylcamitine, Dehydroisoandrosterone 3-sulfate, Metanephrine, PC(32:2), PC(34:2), L-Phenylalanine, Phenylpropiolic acid, Methylmalonic acid, Alpha-ketoisocaproic acid, L-Histidine, L-Methionine, PC(18:1(9Z)/18:1(9Z)), 5,6-trans-25-Hydroxyvitamin D3, 2-Methylcitric acid, Taurine, 1-Pyrroline-5-carboxylic acid, L-Proline, PC(18:0/18:2), 7-Methylguanosine, L-Kynurenine, Beta-Alanine, Xanthosine, PE(34:2), Malonylcarnitine, Gluconic acid, L-Glutamine, Pipecolic acid, Cyclic AMP, L-Valine, Cholesterol, SM(d18:1/26:0), L-Lysine, Carbamoylphosphate, Glycerophosphocholine, Adenylosuccinic acid, and any combination thereof.

8. (canceled)

9. The method of claim 1, wherein the metabolites are selected from the group consisting of formate, glycine, serine, catacholamines, serotonin, glutamate, GABA, vitamin B6, thiamine, folate, vitamin B12, glutathione, cysteine and methionine.

10. (canceled)

11. The method of claim 1, wherein the metabolite is converted to a non-naturally occurring by-product that is analyzed.

12. The method of claim 11, wherein the non-naturally occurring by-product is a mass fragment.

13. (canceled)

14. A method for diagnosing, predicting, or assessing risk of developing a psychiatric or neurological disease or disorder selected from the group consisting of pervasive developmental disorder not otherwise specified, non-verbal learning disabilities, autism, autism spectrum disorders, attention deficit hyperactivity disorder (ADHD), anxiety disorders, post-traumatic stress disorder (PTSD), traumatic brain injury (TBI), social phobia, generalized anxiety disorder, social deficit disorders, schizotypal personality disorder, schizoid personality disorder, schizophrenia, cognitive deficit disorders, dementia, and Alzheimer's Disease in a subject, said method comprising:
detecting an amount of each of a plurality of metabolites in a biological sample obtained from the subject, said plurality of metabolites comprising at least eight (8) metabolites, each of said at least 8 metabolites being in a metabolic pathway selected from the group of pathways consisting of:
a phospholipid metabolic pathway;
a fatty acid oxidation and synthesis metabolic pathway; a purine metabolic pathway;
a bioamine and neurotransmitter metabolic pathway;
a microbiome metabolic pathway;
a sphingolipid metabolic pathway;
a cholesterol, cortisol, and non-gonadal steroid metabolic pathway;
a pyrimidine metabolic pathway;
a 3- and 4-carbon amino acid metabolic pathway;
a branched chain amino acid metabolic pathway;
a tryptophan, kynurenine, serotonin, melatonin metabolic pathway;
a tyrosine and phenylalanine metabolic pathway;
a S-adenosylmethionine (SAM), S-adenosylhomocysteine (SAH), methionine, cysteine, and glutathione metabolic pathway; an eicosanoid and resolvin metabolic pathway;
a pentose phosphate and gluconate metabolic pathway;
a vitamin A and carotenoid metabolic pathway;
a glycolysis metabolic pathway;
a Kreb's cycle metabolic pathway; and
a Vitamin B3 (−Niacin, NAD+) metabolic pathway; and
comparing the amounts so detected with normal or control amounts of the metabolites,
wherein the amounts of the at least 8 metabolites so determined, indicate a likelihood that the subject is at risk of having or developing the disease or disorder.

15. The method of claim 14, wherein each of said 8 metabolites is in a metabolic pathway selected from the group of metabolic pathways consisting of:
a phospholipid metabolic pathway;
a fatty acid oxidation and synthesis metabolic pathway;
a purine metabolic pathway;
a bioamine and neurotransmitter metabolic pathway;
a microbiome metabolic pathway;
a sphingolipid metabolic pathway;
a cholesterol, cortisol, and non-gonadal steroid metabolic pathway;
a pyrimidine metabolic pathway;
a 3- and 4-carbon amino acid metabolic pathway; a branched chain amino acid metabolic pathway;
a tryptophan, kynurenine, serotonin, melatonin metabolic pathway;
a tyrosine and phenylalanine metabolic pathway;
a S-adenosylmethionine (SAM), S-adenosylhomocysteine (SAH), methionine, cysteine, and glutathione metabolic pathway;
an eicosanoid and resolvin metabolic pathway;
a pentose phosphate and gluconate metabolic pathway; and
a vitamin A and carotenoid metabolic pathway.

16. (canceled)

17. The method of claim 15, wherein each of said at least 8 metabolites is in a metabolic pathway selected from the group of metabolic pathways consisting of:
a phospholipid metabolic pathway;
a purine metabolic pathway;
a sphingolipid metabolic pathway;
a cholesterol metabolic pathway;
a pyrimidine metabolic pathway;
a S-adenosylmethionine (SAM), S-adenosylhomocysteine (SAH), methionine, cysteine, and glutathione metabolic pathway;
a microbiome metabolic pathway;
a Kreb's Cycle metabolic pathway;
a glycolysis metabolic pathway; and
a Vitamin B3 (−Niacin, NAD+) metabolic pathway.

18. (canceled)

19. The method of claim 14, wherein each of the at least 8 metabolites is in a metabolic pathway selected from the group of metabolic pathways consisting of:
a phospholipid metabolic pathway;
a purine metabolic pathway;
a sphingolipid metabolic pathway;
a cholesterol cortisol, and/or non-gonadal steroid metabolic pathway;
a pyrimidine metabolic pathway;
a S-adenosylmethionine (SAM), S-adenosylhomocysteine (SAH), methionine, cysteine, and glutathione metabolic pathway; and
a microbiome metabolic pathway.

20-25. (canceled)

26. The method of claim 14, wherein the detection indicates the presence or absence of an alteration in one or more of the group of metabolic pathways,
wherein detection of a reduced amount, compared to a normal or control amount, of two or more metabolites in a pathway or an elevated amount, compared to a normal or control amount, of two or more metabolites in a pathway, indicates an alteration in the pathway.

27-32. (canceled)

33. The method of claim 14, wherein the at least 8 metabolites comprise metabolites selected from the group consisting of: 2-Octenoylcarnitine, Retinol, L-Tryptophan, Nicotinamide N-oxide, Alanine, L-Tyrosine, 3-Hydroxyanthranilic acid, N-Acetyl-L-aspartic acid, Sarcosine, N-Acetylaspartylglutamic acid, Methylcysteine, AICAR, SM(d18:1/12:0), Oleic acid, Docosahexaenoic acid, Glycocholic acid, Guanosine monophosphate, Cytidine, SM(d18:1/22:0 OH), Xanthine, Indoleacrylic acid, 7-ketocholesterol, 3-Hydroxyhexadecanoylcarnitine, Linoleic acid, Adenosine monophosphate, L-Serine, Pantothenic acid, Arachidonic Acid, PC(26:1), Uracil and combinations thereof.

34. The method of claim 33, wherein the at least 8 metabolites further comprise metabolites selected from the group consisting of: PC(30:2), Hypoxanthine,2-Keto-L-gluconate, Glutaconic acid, 5-HETE, PC(28:2), 3-Hydroxyhexadecenoylcarnitine, Hydroxyproline, Dopamine, Myoinositol, 3-Hydroxylinoleylcamitine, PC(30:1), LysoPC(24:0), Indole, SM(d18:1/24:0), PC(28:1), L-Threonine, Mevalonic acid, SM(20:0 OH), Purine ring, 3-Hydroxyisobutyroylcamitine, Dehydroisoandrosterone 3-sulfate, Metanephrine, PC(32:2), PC(34:2), L-Phenylalanine, Phenylpropiolic acid, Methylmalonic acid, Alpha-ketoisocaproic acid, L-Histidine, L-Methionine, PC(18:1(9Z)/18:1(9Z)), 5,6-trans-25-Hydroxyvitamin D3, 2-Methylcitric acid, Taurine, 1-Pyrroline-5-carboxylic acid, L-Proline, PC(18:0/18:2), 7-Methylguanosine, L-Kynurenine, Beta-Alanine, Xanthosine, PE(34:2), Malonylcarnitine, Gluconic acid, L-Glutamine, Pipecolic acid, Cyclic AMP, L-Valine, Cholesterol, SM(d18:1/26:0), L-Lysine, Carbamoylphosphate, Glycerophosphocholine, Adenylosuccinic acid, and combinations thereof.

35-45. (canceled)

46. The method claim 14, wherein an elevation or reduction in the detected amount of metabolite by at least 1%, 5%, 10%, 15%, 20%, 25%, 30%, 40%, 45%, 50%, 55%, 60%, 65%, 70%, 75%, 80%, 85%, or 90% compared to a control or normal amount indicates an elevation or reduction in the metabolite in the sample.

47-50. (canceled)

51. A method of treatment comprising:
performing the method of claim 14, thereby detecting elevated or reduced amounts of one or more of the metabolites compared to a normal or control amounts;
performing a therapy on the subject targeted to the disease or disorder.

52-55. (canceled)
